(19) United States
(12) Patent Application Publication
(10) Pub. No.: US 2026/0055195 A1
(43) Pub. Date: Feb. 26, 2026

(54) TARGETING EPHA3 AND USES THEREOF

(71) Applicant: The Council of the Queensland Institute of Medical Research, Herston, Queensland (AU)

(72) Inventors: Rajiv KHANNA, Herston (AU); Jose Paulo MARTINS, Herston (AU)

(21) Appl. No.: 17/781,271

(22) PCT Filed: Oct. 9, 2020
(86) PCT No.: PCT/AU2020/051090

§ 371 Date: May 31, 2022

(30) Foreign Application Priority Data
Oct. 9, 2019 (AU) 2019903802

(51) Int. Cl. C07K 16/28; A61P 35/00; C07K 14/705; C07K 14/725; C12N 5/0783

(52) U.S. Cl. CPC C07K 16/2866; A61P 35/00; C07K 14/7051; C07K 14/70517; C07K 14/70521; C07K 14/70578; C12N 5/0636; C07K 2317/528; C07K 2317/565; C07K 2317/622; C07K 2317/92; C07K 2319/03; C12N 2510/00

(57) ABSTRACT

Disclosed are antigen-binding molecules and chimeric antigen receptors (CARs) that can at least specifically recognize or bind to EphA3. Also disclosed are methods of medical treatment and prophylaxis.

This application claims priority from Australian Patent Application No. 2019903802, filed 9 Oct. 2019, the contents and elements of which are herein incorporated by reference for all purposes.

### SEQUENCE LISTING

The instant application contains a Sequence Listing which has been submitted via EFS-Web and is hereby incorporated by reference in its entirety. Said ASCII copy, created on Sep. 15, 2025, is named 51619-008001_Sequence_Listing_9_15_25_ST25.txt and is 100,745 bytes in size.

### FIELD OF THE INVENTION

The present invention relates to the fields of molecular biology, and more specifically antibody technology. More particularly, this invention relates to an antibody or chimeric antigen receptor (CAR) that can at least specifically recognize or bind to EphA3. The present invention also relates to methods of medical treatment and prophylaxis.

### BACKGROUND TO THE INVENTION

Ephrin type-A receptor 3 (EphA3) has been found to be over-expressed or aberrantly expressed on tumour cells from a wide variety of human solid tumours and leukemias, including colon cancer, breast cancer, chronic myeloid leukemia (CML) and Glioblastoma multiforme (GBM). GBM is one of the most aggressive solid brain tumours. Standard treatment consists of maximal surgical resection, radiotherapy, and concomitant and adjuvant chemotherapy with temozolomide. However, even with optimal treatment, median survival after initial diagnosis is less than 15 months (1). Recent advances using checkpoint blockade have improved outcomes for several human cancers however GBM seems to be resistant to this treatment approach alone (2). Notwithstanding this, there remains a need for the development of new therapies for not only GBM, but cancer more broadly.

### SUMMARY OF THE INVENTION

The present invention is broadly directed to an anti-EphA3 binding agent, inclusive of a human or humanized, recombinant anti-EphA3 antibody, and methods of using same. A particular form of the invention further provides a chimeric antigen receptor (CAR) comprising an antigen binding domain that can specifically bind EphA3 and methods of using same.

In a broad form, the invention relates to EphA3 binding agents and CARs that comprise one or more CDRs of an EphA3 monoclonal antibody described herein.

In one aspect, the invention provides an EphA3 binding agent comprising at least one complementarity determining region (CDR) having an amino acid sequence set forth in SEQ ID NOs:13-72 and/or Tables 4-7 or an amino acid sequence at least 70% identical thereto.

In some embodiments, the EphA3 binding agent comprises:
(a) a heavy chain immunoglobulin variable region (VH) polypeptide comprising a CDR 1 having an amino acid sequence at least 70% identical to any one of SEQ ID NOs:13-17; a CDR having an amino acid sequence at least 70% identical to any one of SEQ ID NOs: 18 to 22; and a CDR 3 having an amino acid sequence at least 70% identical to any one of SEQ ID NO: 23 to 27; and/or
(b) a light chain immunoglobulin variable region (VL) polypeptide comprising a CDR 1 having an amino acid sequence at least 70% identical to any one of SEQ ID NOs: 28-32; a CDR 2 having an amino acid sequence at least 70% identical to any one of SEQ ID NOs: 33-37; and a CDR 3 having an amino acid sequence at least 70% 30 identical to any one of SEQ ID NOs: 38-42.

With regard to such embodiments, the VH polypeptide suitably comprises an amino acid sequence set forth in SEQ ID NO:153 or an amino acid sequence at least 70% identical thereto; and/or the VL polypeptide suitably comprises an amino acid sequence set forth in SEQ ID NO:154 or an amino acid sequence at least 70% identical thereto.

In some of the same embodiments and some other embodiments, EphA3 binding agent comprises:
(a) a VH polypeptide comprising a CDR 1 having an amino acid sequence at least 70% identical to any one of SEQ ID NOs: 43-47; a CDR 2 having an amino acid sequence at least 70% identical to any one of SEQ ID NOs: 48-52; and a CDR 3 having an amino acid sequence at least 70% identical to any one of SEQ ID NOs: 53-57; and/or
(b) a VL polypeptide comprising a CDR 1 having an amino acid sequence at least 70% identical to any one of SEQ ID NOs: 58-62; a CDR 2 having an amino acid sequence at least 70% identical to any one of SEQ ID NOs: 63-67; and a CDR 3 having an amino acid sequence at least 70% identical to any one of SEQ ID NOs: 68-72.

In this regard, the VH polypeptide can comprise an amino acid sequence set forth in SEQ ID NO:155 or an amino acid sequence at least 70% identical thereto; and/or the VL polypeptide can comprise an amino acid sequence set forth in SEQ ID NO:156 or an amino acid sequence at least 70% identical thereto.

Suitably, the EphA3 binding agent is an antibody or antibody fragment. In one embodiment, the antibody or antibody fragment is a 3C3-1 or 2D4-1 monoclonal antibody or fragment thereof. In certain embodiments, the EphA3 binding agent is a recombinant, human or humanized antibody or antibody fragment.

In another aspect, the invention resides in a chimeric antigen receptor (CAR) comprising an antigen binding domain including at least one CDR having an amino acid sequence set forth in SEQ ID NOs:13-72 and/or Tables 4-7 or an amino acid sequence at least 70% identical thereto, a transmembrane domain, and an intracellular signalling domain.

In some embodiments, the antigen binding domain comprises, consists or consists essentially of:
(a) a heavy chain immunoglobulin variable region (VH) polypeptide comprising a CDR 1 having an amino acid sequence at least 70% identical to any one of SEQ ID NOs:13-17; a CDR having an amino acid sequence at least 70% identical to any one of SEQ ID NOs: 18 to 22; and a CDR 3 having an amino acid sequence at least 70% identical to any one of SEQ ID NO: 23 to 27; and/or
(b) a light chain immunoglobulin variable region (VL) polypeptide comprising a CDR 1 having an amino acid sequence at least 70% identical to any one of SEQ ID NOs: 28-32; a CDR 2 having an amino acid sequence at least 70% identical to any one of SEQ ID NOs: 33-37; and a CDR 3 having an amino acid sequence at least 70% identical to any one of SEQ ID NOs: 38-42.

For such embodiments, the VH polypeptide may comprise an amino acid sequence set forth in SEQ ID NO:153 or an amino acid sequence at least 70% identical thereto; and/or the VL polypeptide may comprise an amino acid sequence set forth in SEQ ID NO:154 or an amino acid sequence at least 70% identical thereto.

In some embodiments, the antigen binding domain comprises, consists or consists essentially of:
(a) a VH polypeptide comprising a CDR 1 having an amino acid sequence at least 70% identical to any one of SEQ ID NOs: 43-47; a CDR 2 having an amino acid sequence at least 70% identical to any one of SEQ ID NOs: 48-52; and a CDR 3 having an amino acid sequence at least 70% identical to any one of SEQ ID NOs: 53-57; and/or
(b) a VL polypeptide comprising a CDR 1 having an amino acid sequence at least 70% identical to any one of SEQ ID NOs: 58-62; a CDR 2 having an amino acid sequence at least 70% identical to any one of SEQ ID NOs: 63-67; and a CDR 3 having an amino acid sequence at least 70% identical to any one of SEQ ID NOs: 68-72.

For such embodiments, the VH polypeptide may comprise an amino acid sequence set forth in SEQ ID NO:155 or an amino acid sequence at least 70% identical thereto; and/or the VL polypeptide may comprise an amino acid sequence set forth in SEQ ID NO:156 or an amino acid sequence at least 70% identical thereto.

In some embodiments, the antigen binding domain comprises a linker. In one particular embodiment, the linker comprises, consists or consists essentially of an amino acid sequence set forth in SEQ ID NO: 158 or an amino acid sequence at least 70% identical thereto.

Suitably, the CAR further comprises a leader or signal peptide sequence, such as the leader or signal peptide sequence of CD8 set forth in SEQ ID NO: 157 or an amino acid sequence at least 70% identical thereto.

In certain embodiments, the transmembrane domain comprises a CD8 transmembrane domain, such as the CD8 transmembrane domain that comprises an amino acid sequence set forth in SEQ ID NO:159 or an amino acid sequence at least 70% identical thereto.

Suitably, the intracellular T cell signalling domain comprises a CD3 zeta intracellular signalling domain.

In particular embodiments, the intracellular signalling domain comprises a CD3 amino acid sequence set forth in SEQ ID NO:162 or an amino acid sequence at least 70% identical thereto.

Suitably, the CAR further comprises one or more co-stimulatory domains, such as a CD28 co-stimulatory domain having the amino acid sequence set forth in SEQ ID NO:161 or an amino acid sequence at least 70% identical thereto and/or a CD137 co-stimulatory domain having the amino acid sequence set forth in SEQ ID NO:160 or an amino acid sequence at least 70% identical thereto.

In some embodiments, the EphA3 binding agent of the first aspect or the CAR of the second aspect is for use in the treatment or prevention of a cancer, such as a solid cancer like glioblastoma multiforme, in a subject.

In this regard, the antibodies and antigen-binding molecules described above and elsewhere here in display substantial anti-tumour activity, and are particularly effective at decreasing. In some embodiments, the antigen-binding molecules of the invention have the capability of substantially eliminating a tumour from a subject with cancer.

In another aspect, the invention provides an isolated nucleic acid encoding the EphA3 binding agents and/or the CAR as described above and elsewhere herein.

In yet another aspect, the invention resides in a genetic construct comprising the isolated nucleic acids described above and elsewhere herein.

In still another aspect, the invention provides a host cell comprising the nucleic acids and/or the genetic constructs described above or elsewhere herein.

Suitably, the host cell is or comprises a T-cell.

In another aspect, the invention resides in a method of producing an isolated EphA3 binding agent or CAR, said method including the steps of; (i) culturing the host cell of the fifth aspect; and (ii) isolating said EphA3 binding agent or CAR from said host cell cultured in step (i).

In yet another aspect, the invention provides an EphA3 binding agent or CAR produced by the method of the sixth aspect.

In yet another aspect, the invention resides in an antibody or antibody fragment which binds and/or is raised against:
(i) the EphA3 binding agent of the first aspect; and/or
(ii) the CAR of the second aspect.

In another aspect, the invention provides a composition comprising the EphA3 binding agent of the first or sixth aspects, the CAR of the second or sixth aspects, the nucleic acid of the third aspect, the genetic construct of the fourth aspect and/or the host cell of the fifth aspect and a pharmaceutically acceptable carrier diluent or excipient.

In still yet another aspect, the invention resides in a method of treating or preventing a cancer in a subject, said method including the step of administering a therapeutically effective amount of the EphA3 binding agent of the first or sixth aspects, the CAR of the second or sixth aspects, the nucleic acid of the third aspect, the genetic construct of the fourth aspect, the host cell of the fifth aspect and/or the composition of the ninth aspect to the subject to thereby treat or prevent the cancer in the subject.

In another aspect, the invention provides use of the EphA3 binding agent of the first or sixth aspects, the CAR of the second or sixth aspects, the nucleic acid of the third aspect, the genetic construct of the fourth aspect and/or the host cell of the fifth aspect in the manufacture of a medicament for the prevention and/or treatment of a cancer in a subject.

With respect to the first, second, tenth and eleventh aspects, the cancer suitably is or comprises glioblastoma multiforme.

In another aspect, the invention resides in a method of detecting EphA3 or a cell expressing EphA3, said method including the step of forming a complex between the EphA3 binding agent of the first aspect, or the CAR of the second aspect and EphA3 to thereby detect EphA3 or the cell expressing EphA3.

Suitably, the present method includes the initial step of contacting EphA3 or the cell expressing EphA3 with the EphA3 binding agent or the CAR.

In certain embodiments, the cell is or comprises a cancer cell.

In yet another aspect, the invention provides an isolated protein comprising, consisting essentially of or consisting of an amino acid sequence set forth in any one of SEQ ID NOS:13 to 156 and/or Tables 4-7 or an amino acid sequence at least 70% identical thereto.

In still yet another aspect the invention provides a human T-cell expressing: (a) a T-cell receptor (TCR) that is activated by binding to a CMV antigen; and (b) a chimeric antigen receptor (CAR) comprising an antigen-binding domain that binds to an epitope on EphA3.

In some embodiments, the antigen-binding domain is a scFv comprising a heavy chain variable (VH) region and a light chain variable (VL) region.

In a similar aspect, the invention provides a T-cell that comprises (a) a T-cell receptor (TCR) that expresses a TCR that is specific for a CMV antigen; and (b) an antigen-binding molecule that binds to EphA3.

In still another aspect, the invention resides in an isolated nucleic acid comprising, consisting or consisting essentially of a nucleic acid sequence set forth in any one of SEQ ID NOS: 1 to 12 and/or Table 3 or a nucleic acid sequence at least 70% identical thereto.

Throughout this specification, unless the context requires otherwise, the words “comprise”, “comprises” and “comprising” will be understood to imply the inclusion of a stated integer or group of integers but not the exclusion of any other integer or group of integers.

By “consists essentially of” in the context of an amino acid sequence means that the recited amino acid sequences includes an additional 1, 2, 3, 4, or 5 amino acids at an N- and/or C-terminus thereof.

As used herein, the indefinite articles ‘a’ and ‘an’ are used here to refer to or encompass singular or plural elements or features and should not be taken as meaning or defining “one” or a “single” element or feature.

### BRIEF DESCRIPTION OF THE FIGURES

FIG. 1—Cloning strategy for EphA3 (P29320|21-541) pcDNA3.4 for Expi293F cell expression. The protein sequence (SEQ ID NO: 213) and the DNA sequence (SEQ ID NO: 214) are shown.

FIG. 2—SDS-PAGE and Western blot analysis of EphA3 (P29320121-541). Lane M1: Protein marker TaKaRa, (Cat No. 3452); Lane M2: Protein marker (GenScript, Cat No. M00521); Lane 1: Reducing condition; Lane 2 Non-reducing condition; Lane P: Multiple-tag (GenScript, Cat No. M0101) as a positive control; Primary antibody: Mouse-anti-His mAb (GenScript, Cat No. A00186).

FIG. 3—Parental antibody clones selected for subcloning (Panel A). Mice were immunized with a recombinant human EphA3 protein (PP29320121-541). Hybridomas were generated and 5 parental hybridoma clones were selected for subcloning based on EphA3 specificity by ELISA and FACS with LK63 tumour cells expressing EphA3 (Panel B).

FIG. 4—Monoclonal antibodies generated for EphA3 have different binding efficiencies. Subclone supernatants were screened for EphA3 binding efficiency by ELISA (Panel A) and FACS (Panel B).

FIG. 5—EphA3 is expressed on glioma cell lines. Flow cytometric analysis using 3C3-1 anti-EphA3 on cultured U87, D270 and U251 cell lines.

FIG. 6—Schematic representation of (top) pD2109-FA301_Ires-RFP and (bottom) pD2109-FA302_Ires-RFP CAR constructs with 3C3-1 scFv binding regions and CD28ζ or 4-1 BBC signalling domains.

FIG. 7—RT-PCR and agarose gel electrophoresis of the sequence spanning the CAR T fragment (546 bp) in HEK293T cells. Fw—CAGCGGCTACACCTTTACCA [SEQ ID NO: 215] and Rev—CCGGAGAATCTATCCGGCAC [SEQ ID NO: 216] primers.

FIG. 8—Transduction of Jurkat cells with a EphA3-CAR lentivirus generated for constructs FA301 and FA302 (RFP reporter) and pD2109 (GFP reporter).

FIG. 9—Surface expression of EphA3-CAR in Jurkat cells. Cells were transduced with (left) FA301 and (right) FA302 lentiviruses and incubated with plate-bound EphA3-his protein. Cells were stained with or without aEphA3 primary Ab, followed by αHis-tag Ab to determine EphA3-CAR surface expression.

FIG. 10—CAR-expressing Jurkat cells are activated by EphA3. FA301 and FA302 transduced Jurkat cells were incubated with increasing concentrations of plate-bound EphA3 protein. Cells were stained for CD69 expression by FACs and levels of RFP positive, expressing the CAR, were compared to RFP negative cells.

FIG. 11—CAR-expressing Jurkat cells are activated by a EphA3 expressing tumour cell line. FA301 transduced Jurkat cells were incubated with Lk63 cells at 1:10 ratio (Jurkat-CAR: Lk63). Jurkat cells were stained for CD69 expression by FACs and levels on RFP positive cells were compared to RFP negative cells.

FIG. 12—CMV expanded T-cells were transduced the pD2109 and FA301 lentiviruses. Transduction efficiency was determined by FACS after 3 days.

FIG. 13—In vitro comparison of EphA3 CAR T-cells co-stimulation domains. (A) Peripheral blood mononuclear cells were stimulated using CD3/28⁺ beads (polyclonal expansion) and cells were transduced with EphA3 lentivirus FA305-BBζ or FA306-28ζ and cultured for 12 days. Non-transduced (NT) T-cells were maintained as control. The CAR expression was assessed by surface expression of anti-mouse IgG (CAR) and analysis by FACS. (B) Characterisation of EphA3 CAR T-cell effector function. CAR transduced T-cells were incubated with LK63 (EphA3+) target cells overnight and functionality was examined using intracellular TNF.

FIG. 14—Generation of CAR T cells. Peripheral blood mononuclear cells were stimulated using CD3/28⁺ beads (polyclonal expansion) or a pool of 26 HLA class I and class II-restricted T-cell peptide epitopes from multiple CMV antigens. These cells were transduced with EphA3 lentivirus and cultured for 14 days. Non-transduced (NT) T-cells were maintained as control. The expression of CAR and CMV-specificity was assessed by FACS analysis for anti-mouse IgG (CAR) and HLA complex—peptide tetramers for CMV (VTE and ELK).

FIG. 15—Comparison of EphA3 CAR T-cell effector function and cytotoxicity in polyclonal and CMV-specific T-cells. (A) CAR transduced T-cells were incubated with LK63 (EphA3+) target cells overnight and functionality was examined using intracellular IFN-γ, TNF and the cell surface mobilization of CD107a.

FIG. 16—Characterisation of EphA3 CAR T-cell in vitro cytotoxicity. (A) The ability of T-cells expressing EphA3-CAR to eliminate specifically EphA3+ tumours was measured by real-time target-induced cytolysis of U251 (EphA3+) and U87 (EphA3−) glioma cell lines. (B & C) RTCA analysis of polyclonal and CMV-specific EphA3-CARs at 1:1, 5:1 and 10:1 effector to target ratios using the U251 target cell line.

FIG. 17—EphA3 CAR T-cells mediate a potent anti-GBM response in a xenograft model of GBM. (A) Schematic representation of experiment design. NRG mice were transplanted with luciferase-expressing glioma cell lines U251 (EphA3+) or U87 (EphA3−) subcutaneously in the flank (heterotopic model). Tumour size was measured or determined by bioluminescence. Once tumours reached approximately 25 mm², the mice received intravenous EphA3-CAR, NT (non-transduced) T-cells or CAR19 (non-specific CAR T-cells). Representative FACS plots analysis of (B) CD4+ and CD8+ percentages and (C) Ki67 expression in blood collected at day 17. (D) U251 tumour burden was determined weekly to assess the impact of the CAR-T cell therapy on tumour regression. (E) Comparison of CAR EphA3 treatment in U251 and U87 bearing mice (F) In vivo imaging of U251 (left) and U87 (right) luminescent tumour xenograft mice. (G) Kaplan-Meier survival curve for mice which received EphA3-CAR treatment or control cells (NT or CAR19).

BRIEF DESCRIPTION OF THE SEQUENCES

| | |
|---|---|
| SEQ ID NO: 1 | Clone 3C3-1 Heavy Chain CDR1 nucleotide sequence |
| SEQ ID NO: 2 | Clone 3C3-1 Heavy Chain CDR2 nucleotide sequence |
| SEQ ID NO: 3 | Clone 3C3-1 Heavy Chain CDR3 nucleotide sequence |
| SEQ ID NO: 4 | Clone 3C3-1 Light Chain CDR1 nucleotide sequence |
| SEQ ID NO: 5 | Clone 3C3-1 Light Chain CDR2 nucleotide sequence |
| SEQ ID NO: 6 | Clone 3C3-1 Light Chain CDR3 nucleotide sequence |
| SEQ ID NO: 7 | Clone 2D4-1 Heavy Chain CDR1 nucleotide sequence |
| SEQ ID NO: 8 | Clone 2D4-1 Heavy Chain CDR2 nucleotide sequence |
| SEQ ID NO: 9 | Clone 2D4-1 Heavy Chain CDR3 nucleotide sequence |
| SEQ ID NO: 10 | Clone 2D4-1 Light Chain CDR1 nucleotide sequence |
| SEQ ID NO: 11 | Clone 2D4-1 Light Chain CDR2 nucleotide sequence |
| SEQ ID NO: 12 | Clone 2D4-1 Light Chain CDR3 nucleotide sequence |
| SEQ ID NO: 13 | Clone 3C3-1 Heavy Chain CDR1 amino acid sequence (Chothia) |
| SEQ ID NO: 14 | Clone 3C3-1 Heavy Chain CDR1 amino acid sequence (AbM) |
| SEQ ID NO: 15 | Clone 3C3-1 Heavy Chain CDR1 amino acid sequence (Kabat) |
| SEQ ID NO: 16 | Clone 3C3-1 Heavy Chain CDR1 amino acid sequence (Contact) |
| SEQ ID NO: 17 | Clone 3C3-1 Heavy Chain CDR1 amino acid sequence (IMGT) |
| SEQ ID NO: 18 | Clone 3C3-1 Heavy Chain CDR2 amino acid sequence (Chothia) |
| SEQ ID NO: 19 | Clone 3C3-1 Heavy Chain CDR2 amino acid sequence (AbM) |
| SEQ ID NO: 20 | Clone 3C3-1 Heavy Chain CDR2 amino acid sequence (Kabat) |
| SEQ ID NO: 21 | Clone 3C3-1 Heavy Chain CDR2 amino acid sequence (Contact) |
| SEQ ID NO: 22 | Clone 3C3-1 Heavy Chain CDR2 amino acid sequence (IMGT) |
| SEQ ID NO: 23 | Clone 3C3-1 Heavy Chain CDR3 amino acid sequence (Chothia) |
| SEQ ID NO: 24 | Clone 3C3-1 Heavy Chain CDR3 amino acid sequence (AbM) |
| SEQ ID NO: 25 | Clone 3C3-1 Heavy Chain CDR3 amino acid sequence (Kabat) |
| SEQ ID NO: 26 | Clone 3C3-1 Heavy Chain CDR3 amino acid sequence (Contact) |
| SEQ ID NO: 27 | Clone 3C3-1 Heavy Chain CDR3 amino acid sequence (IMGT) |
| SEQ ID NO: 28 | Clone 3C3-1 Light Chain CDR1 amino acid sequence (Chothia) |
| SEQ ID NO: 29 | Clone 3C3-1 Light Chain CDR1 amino acid sequence (AbM) |
| SEQ ID NO: 30 | Clone 3C3-1 Light Chain CDR1 amino acid sequence (Kabat) |
| SEQ ID NO: 31 | Clone 3C3-1 Light Chain CDR1 amino acid sequence (Contact) |
| SEQ ID NO: 32 | Clone 3C3-1 Light Chain CDR1 amino acid sequence (IMGT) |
| SEQ ID NO: 33 | Clone 3C3-1 Light Chain CDR2 amino acid sequence (Chothia) |
| SEQ ID NO: 34 | Clone 3C3-1 Light Chain CDR2 amino acid sequence (AbM) |
| SEQ ID NO: 35 | Clone 3C3-1 Light Chain CDR2 amino acid sequence (Kabat) |
| SEQ ID NO: 36 | Clone 3C3-1 Light Chain CDR2 amino acid sequence (Contact) |
| SEQ ID NO: 37 | Clone 3C3-1 Light Chain CDR2 amino acid sequence (IMGT) |
| SEQ ID NO: 38 | Clone 3C3-1 Light Chain CDR3 amino acid sequence (Chothia) |
| SEQ ID NO: 39 | Clone 3C3-1 Light Chain CDR3 amino acid sequence (AbM) |
| SEQ ID NO: 40 | Clone 3C3-1 Light Chain CDR3 amino acid sequence (Kabat) |
| SEQ ID NO: 41 | Clone 3C3-1 Light Chain CDR3 amino acid sequence (Contact) |
| SEQ ID NO: 42 | Clone 3C3-1 Light Chain CDR3 amino acid sequence (IMGT) |
| SEQ ID NO: 43 | Clone 2D4-1 Heavy Chain CDR1 amino acid sequence (Chothia) |
| SEQ ID NO: 44 | Clone 2D4-1 Heavy Chain CDR1 amino acid sequence (AbM) |
| SEQ ID NO: 45 | Clone 2D4-1 Heavy Chain CDR1 amino acid sequence (Kabat) |
| SEQ ID NO: 46 | Clone 2D4-1 Heavy Chain CDR1 amino acid sequence (Contact) |
| SEQ ID NO: 47 | Clone 2D4-1 Heavy Chain CDR1 amino acid sequence (IMGT) |
| SEQ ID NO: 48 | Clone 2D4-1 Heavy Chain CDR2 amino acid sequence (Chothia) |
| SEQ ID NO: 49 | Clone 2D4-1 Heavy Chain CDR2 amino acid sequence (AbM) |
| SEQ ID NO: 50 | Clone 2D4-1 Heavy Chain CDR2 amino acid sequence (Kabat) |
| SEQ ID NO: 51 | Clone 2D4-1 Heavy Chain CDR2 amino acid sequence (Contact) |
| SEQ ID NO: 52 | Clone 2D4-1 Heavy Chain CDR2 amino acid sequence (IMGT) |
| SEQ ID NO: 53 | Clone 2D4-1 Heavy Chain CDR3 amino acid sequence (Chothia) |
| SEQ ID NO: 54 | Clone 2D4-1 Heavy Chain CDR3 amino acid sequence (AbM) |
| SEQ ID NO: 55 | Clone 2D4-1 Heavy Chain CDR3 amino acid sequence (Kabat) |
| SEQ ID NO: 56 | Clone 2D4-1 Heavy Chain CDR3 amino acid sequence (Contact) |
| SEQ ID NO: 57 | Clone 2D4-1 Heavy Chain CDR3 amino acid sequence (IMGT) |
| SEQ ID NO: 58 | Clone 2D4-1 Light Chain CDR1 amino acid sequence (Chothia) |
| SEQ ID NO: 59 | Clone 2D4-1 Light Chain CDR1 amino acid sequence (AbM) |
| SEQ ID NO: 60 | Clone 2D4-1 Light Chain CDR1 amino acid sequence (Kabat) |
| SEQ ID NO: 61 | Clone 2D4-1 Light Chain CDR1 amino acid sequence (Contact) |
| SEQ ID NO: 62 | Clone 2D4-1 Light Chain CDR1 amino acid sequence (IMGT) |
| SEQ ID NO: 63 | Clone 2D4-1 Light Chain CDR2 amino acid sequence (Chothia) |
| SEQ ID NO: 64 | Clone 2D4-1 Light Chain CDR2 amino acid sequence (AbM) |
| SEQ ID NO: 65 | Clone 2D4-1 Light Chain CDR2 amino acid sequence (Kabat) |
| SEQ ID NO: 66 | Clone 2D4-1 Light Chain CDR2 amino acid sequence (Contact) |
| SEQ ID NO: 67 | Clone 2D4-1 Light Chain CDR2 amino acid sequence (IMGT) |
| SEQ ID NO: 68 | Clone 2D4-1 Light Chain CDR3 amino acid sequence (Chothia) |
| SEQ ID NO: 69 | Clone 2D4-1 Light Chain CDR3 amino acid sequence (AbM) |
| SEQ ID NO: 70 | Clone 2D4-1 Light Chain CDR3 amino acid sequence (Kabat) |
| SEQ ID NO: 71 | Clone 2D4-1 Light Chain CDR3 amino acid sequence (Contact) |
| SEQ ID NO: 72 | Clone 2D4-1 Light Chain CDR3 amino acid sequence (IMGT) |
| SEQ ID NO: 73 | Clone 3C3-1 HFR1 amino acid sequence (Chothia) |
| SEQ ID NO: 74 | Clone 3C3-1 HFR1 amino acid sequence (AbM) |
| SEQ ID NO: 75 | Clone 3C3-1 HFR1 amino acid sequence (Kabat) |
| SEQ ID NO: 76 | Clone 3C3-1 HFR1 amino acid sequence (Contact) |
| SEQ ID NO: 77 | Clone 3C3-1 HFR1 amino acid sequence (IMGT) |
| SEQ ID NO: 78 | Clone 3C3-1 HFR2 amino acid sequence (Chothia) |
| SEQ ID NO: 79 | Clone 3C3-1 HFR2 amino acid sequence (AbM) |
| SEQ ID NO: 80 | Clone 3C3-1 HFR2 amino acid sequence (Kabat) |
| SEQ ID NO: 81 | Clone 3C3-1 HFR2 amino acid sequence (Contact) |
| SEQ ID NO: 82 | Clone 3C3-1 HFR2 amino acid sequence (IMGT) |
| SEQ ID NO: 83 | Clone 3C3-1 HFR3 amino acid sequence (Chothia) |
| SEQ ID NO: 84 | Clone 3C3-1 HFR3 amino acid sequence (AbM) |
| SEQ ID NO: 85 | Clone 3C3-1 HFR3 amino acid sequence (Kabat) |
| SEQ ID NO: 86 | Clone 3C3-1 HFR3 amino acid sequence (Contact) |
| SEQ ID NO: 87 | Clone 3C3-1 HFR3 amino acid sequence (IMGT) |
| SEQ ID NO: 88 | Clone 3C3-1 HFR4 amino acid sequence (Chothia) |
| SEQ ID NO: 89 | Clone 3C3-1 HFR4 amino acid sequence (AbM) |
| SEQ ID NO: 90 | Clone 3C3-1 HFR4 amino acid sequence (Kabat) |
| SEQ ID NO: 91 | Clone 3C3-1 HFR4 amino acid sequence (Contact) |
| SEQ ID NO: 92 | Clone 3C3-1 HFR4 amino acid sequence (IMGT) |
| SEQ ID NO: 93 | Clone 3C3-1 LFR1 amino acid sequence (Chothia) |
| SEQ ID NO: 94 | Clone 3C3-1 LFR1 amino acid sequence (AbM) |
| SEQ ID NO: 95 | Clone 3C3-1 LFR1 amino acid sequence (Kabat) |
| SEQ ID NO: 96 | Clone 3C3-1 LFR1 amino acid sequence (Contact) |
| SEQ ID NO: 97 | Clone 3C3-1 LFR1 amino acid sequence (IMGT) |
| SEQ ID NO: 98 | Clone 3C3-1 LFR2 amino acid sequence (Chothia) |
| SEQ ID NO: 99 | Clone 3C3-1 LFR2 amino acid sequence (AbM) |
| SEQ ID NO: 100 | Clone 3C3-1 LFR2 amino acid sequence (Kabat) |
| SEQ ID NO: 101 | Clone 3C3-1 LFR2 amino acid sequence (Contact) |
| SEQ ID NO: 102 | Clone 3C3-1 LFR2 amino acid sequence (IMGT) |
| SEQ ID NO: 103 | Clone 3C3-1 LFR3 amino acid sequence (Chothia) |
| SEQ ID NO: 104 | Clone 3C3-1 LFR3 amino acid sequence (AbM) |
| SEQ ID NO: 105 | Clone 3C3-1 LFR3 amino acid sequence (Kabat) |
| SEQ ID NO: 106 | Clone 3C3-1 LFR3 amino acid sequence (Contact) |
| SEQ ID NO: 107 | Clone 3C3-1 LFR3 amino acid sequence (IMGT) |
| SEQ ID NO: 108 | Clone 3C3-1 LFR4 amino acid sequence (Chothia) |
| SEQ ID NO: 109 | Clone 3C3-1 LFR4 amino acid sequence (AbM) |
| SEQ ID NO: 110 | Clone 3C3-1 LFR4 amino acid sequence (Kabat) |
| SEQ ID NO: 111 | Clone 3C3-1 LFR4 amino acid sequence (Contact) |
| SEQ ID NO: 112 | Clone 3C3-1 LFR4 amino acid sequence (IMGT) |
| SEQ ID NO: 113 | Clone 2D4-1 HFR1 amino acid sequence (Chothia) |
| SEQ ID NO: 114 | Clone 2D4-1 HFR1 amino acid sequence (AbM) |
| SEQ ID NO: 115 | Clone 2D4-1 HFR1 amino acid sequence (Kabat) |
| SEQ ID NO: 116 | Clone 2D4-1 HFR1 amino acid sequence (Contact) |
| SEQ ID NO: 117 | Clone 2D4-1 HFR1 amino acid sequence (IMGT) |
| SEQ ID NO: 118 | Clone 2D4-1 HFR2 amino acid sequence (Chothia) |
| SEQ ID NO: 119 | Clone 2D4-1 HFR2 amino acid sequence (AbM) |
| SEQ ID NO: 120 | Clone 2D4-1 HFR2 amino acid sequence (Kabat) |
| SEQ ID NO: 121 | Clone 2D4-1 HFR2 amino acid sequence (Contact) |
| SEQ ID NO: 122 | Clone 2D4-1 HFR2 amino acid sequence (IMGT) |
| SEQ ID NO: 123 | Clone 2D4-1 HFR3 amino acid sequence (Chothia) |
| SEQ ID NO: 124 | Clone 2D4-1 HFR3 amino acid sequence (AbM) |
| SEQ ID NO: 125 | Clone 2D4-1 HFR3 amino acid sequence (Kabat) |
| SEQ ID NO: 126 | Clone 2D4-1 HFR3 amino acid sequence (Contact) |
| SEQ ID NO: 127 | Clone 2D4-1 HFR3 amino acid sequence (IMGT) |
| SEQ ID NO: 128 | Clone 2D4-1 HFR4 amino acid sequence (Chothia) |
| SEQ ID NO: 129 | Clone 2D4-1 HFR4 amino acid sequence (AbM) |
| SEQ ID NO: 130 | Clone 2D4-1 HFR4 amino acid sequence (Kabat) |
| SEQ ID NO: 131 | Clone 2D4-1 HFR4 amino acid sequence (Contact) |
| SEQ ID NO: 132 | Clone 2D4-1 HFR4 amino acid sequence (IMGT) |
| SEQ ID NO: 133 | Clone 2D4-1 LFR1 amino acid sequence (Chothia) |
| SEQ ID NO: 134 | Clone 2D4-1 LFR1 amino acid sequence (AbM) |
| SEQ ID NO: 135 | Clone 2D4-1 LFR1 amino acid sequence (Kabat) |
| SEQ ID NO: 136 | Clone 2D4-1 LFR1 amino acid sequence (Contact) |
| SEQ ID NO: 137 | Clone 2D4-1 LFR1 amino acid sequence (IMGT) |
| SEQ ID NO: 138 | Clone 2D4-1 LFR2 amino acid sequence (Chothia) |
| SEQ ID NO: 139 | Clone 2D4-1 LFR2 amino acid sequence (AbM) |
| SEQ ID NO: 140 | Clone 2D4-1 LFR2 amino acid sequence (Kabat) |
| SEQ ID NO: 141 | Clone 2D4-1 LFR2 amino acid sequence (Contact) |
| SEQ ID NO: 142 | Clone 2D4-1 LFR2 amino acid sequence (IMGT) |
| SEQ ID NO: 143 | Clone 2D4-1 LFR3 amino acid sequence (Chothia) |
| SEQ ID NO: 144 | Clone 2D4-1 LFR3 amino acid sequence (AbM) |
| SEQ ID NO: 145 | Clone 2D4-1 LFR3 amino acid sequence (Kabat) |
| SEQ ID NO: 146 | Clone 2D4-1 LFR3 amino acid sequence (Contact) |
| SEQ ID NO: 147 | Clone 2D4-1 LFR3 amino acid sequence (IMGT) |
| SEQ ID NO: 148 | Clone 2D4-1 LFR4 amino acid sequence (Chothia) |
| SEQ ID NO: 149 | Clone 2D4-1 LFR4 amino acid sequence (AbM) |
| SEQ ID NO: 150 | Clone 2D4-1 LFR4 amino acid sequence (Kabat) |
| SEQ ID NO: 151 | Clone 2D4-1 LFR4 amino acid sequence (Contact) |
| SEQ ID NO: 152 | Clone 2D4-1 LFR4 amino acid sequence (IMGT) |
| SEQ ID NO: 153 | Clone 3C3-1 Heavy Chain amino acid sequence |
| SEQ ID NO: 154 | Clone 3C3-1 Light Chain amino acid sequence |
| SEQ ID NO: 155 | Clone 2D4-1 Heavy Chain amino acid sequence |
| SEQ ID NO: 156 | Clone 2D4-1 Light Chain amino acid sequence |
| SEQ ID NO: 157 | CD8 signal peptide sequence |
| SEQ ID NO: 158 | Spacer/linker amino acid sequence |
| SEQ ID NO: 159 | CD8 hinge and transmembrane amino acid sequence |
| SEQ ID NO: 160 | 4-1BB/CD137 co-stimulatory domain |
| SEQ ID NO: 161 | CD28 co-stimulatory domain |
| SEQ ID NO: 162 | CD3-ζ intracellular signalling domain |
| SEQ ID NO: 163 | IRES nucleic acid sequence |
| SEQ ID NO: 164 | M_Cayenne RFP amino acid sequence |
| SEQ ID NO: 165 | Human EphA3 amino acid sequence (precursor) |
| SEQ ID NO: 166 | Human EphA3 mature amino acid sequence |
| SEQ ID NO: 167 | Human EphA3 extracellular domain amino acid sequence |
| SEQ ID NO: 168 | Human EphA3 transmembrane domain amino acid sequence |
| SEQ ID NO: 169 | Human EphA3 cytoplasmic domain amino acid sequence |
| SEQ ID NO: 170 | Human EphA3 Eph ligand-binding domain amino acid sequence |
| SEQ ID NO: 171 | Human EphA3 fibronectin type-III domain amino acid sequence |
| SEQ ID NO: 172 | Human EphA3 fibronectin type-III domain amino acid sequence |
| SEQ ID NO: 173 | Human EphA3 protein kinase domain amino acid sequence |
| SEQ ID NO: 174 | Human EphA3 sterile alpha motif amino acid sequence |

### DETAILED DESCRIPTION OF THE INVENTION

The present invention is at least partly based on the production of monoclonal antibodies directed to EphA3 and the subsequent creation of chimeric antigen receptors (CARs) based on the binding domains of these monoclonal antibodies. These monoclonal antibodies may be particularly suitable for the treatment and/or prevention of cancer, such as glioblastoma multiforme. Additionally, T cells expressing these CARs may be suitable for adoptive immunotherapy in subjects with cancer.

The present invention relates to novel EphA3 binding molecules having novel and/or improved properties as compared to known anti-EphA3 antibodies. In one aspect, the invention provides novel EphA3 binding molecules comprising at least one complementarity determining region (CDR) having an amino acid sequence set forth in any one of SEQ ID NOs: 13-72 and/or Tables 2-5 or an amino acid sequence at least 70% identical thereto.

### EphA3

Ephrin type-A receptor 3 (EphA3; also referred to e.g., as EPH receptor A3; EPH-like kinase 4; human embryo kinase; tyrosine-protein kinase TYRO4; and tyrosine-protein kinase receptor ETK1) includes all known and naturally occurring EphA3 molecules inclusive of full length EphA3 protein and fragments, variants and derivatives thereof. EphA3 includes, but is not limited to, mammalian EphA3, such as human EphA3 as identified by UniProtKB Accession No. P29320 (as set forth in SEQ ID NO: 165). In humans, EphA3 is encoded by the EPHA3 gene (also known as ETK, ETK1, HEK, and TYRO4). The function of EphA3 is described e.g., in Boyd et al., J Biol Chem, 267(5): 3262-3267, which is hereby incorporated by reference in its entirety. EphA3 is a ˜110 kDa single-pass type I transmembrane protein that functions as a receptor tyrosine kinase which binds promiscuously membrane-bound ephrin family ligands residing on adjacent cells, leading to contact-dependent bidirectional signalling into neighbouring cells.

The N-terminal 20 amino acids of SEQ ID NO: 165 constitutes a signal peptide, and so the mature form of EphA3 (i.e., after processing to remove the signal peptide) has the amino acid shown in SEQ ID NO: 166. Positions 21 to 541 of SEQ ID NO: 165 form the extracellular domain (SEQ ID NO: 167), positions 542 to 565 form a transmembrane domain (SEQ ID NO: 168), and positions 566 to 983 form the cytoplasmic domain (SEQ ID NO: 169). The extracellular domain comprises an Eph ligand-binding domain (positions 29 to 207 of SEQ ID NO: 165, shown in SEQ ID NO: 170); and two fibronectin type-III domains (positions 325 to 435 of SEQ ID NO: 165, and positions 436 to 531 of SEQ ID NO: 165, shown in SEQ ID NO: 171 and 172, respectively). The cytoplasmic domain comprises a protein kinase domain (at position 621 to 882 of SEQ ID NO: 165, shown in SEQ ID NO: 173). The cytoplasmic domain also comprises a sterile alpha motif (SAM) (positions 911 to 975 of SEQ ID NO: 165, shown in SEQ ID NO: 174).

EphA3 mature amino acid sequence:
[SEQ ID NO: 165]
MDCQLSILLLLSCSVLDSFGELIPQPSNEVNLLDSKTIQGELGWISYP
SHGWEEISGVDEHYTPIRTYQVCNVMDHSQNNWLRTNWVPRNSAQKIY
VELKFTLRDCNSIPLVLGTCKETFNLYYMESDDDHGVKFREHQFTKID
TIAADESFTQMDLGDRILKLNTEIREVGPVNKKGFYLAFQDVGACVAL
VSVRVYFKKCPFTVKNLAMFPDTVPMDSQSLVEVRGSCVNNSKEEDPP
RMYCSTEGEWLVPIGKCSCNAGYEERGFMCQACRPGFYKALDGNMKCA
KCPPHSSTQEDGSMNCRCENNYFRADKDPPSMACTRPPSSPRNVISNI
NETSVILDWSWPLDTGGRKDVTFNIICKKCGWNIKQCEPCSPNVRFLP
RQFGLTNTTVTVTDLLAHTNYTFEIDAVNGVSELSSPPRQFAAVSITT
NQAAPSPVLTIKKDRTSRNSISLSWQEPEHPNGIILDYEVKYYEKQEQ
ETSYTILRARGTNVTISSLKPDTIYVFQIRARTAAGYGTNSRKFEFET
SPDSFSISGESSQVVMIAISAAVAIILLTVVIYVLIGRFCGYKSKHGA
DEKRLHFGNGHLKLPGLRTYVDPHTYEDPTQAVHEFAKELDATNISID
KVVGAGEFGEVCSGRLKLPSKKEISVAIKTLKVGYTEKQRRDFLGEAS
IMGQFDHPNIIRLEGVVTKSKPVMIVTEYMENGSLDSFLRKHDAQFTV
IQLVGMLRGIASGMKYLSDMGYVHRDLAARNILINSNLVCKVSDFGLS
RVLEDDPEAAYTTRGGKIPIRWTSPEAIAYRKFTSASDVWSYGIVLWE
VMSYGERPYWEMSNQDVIKAVDEGYRLPPPMDCPAALYQLMLDCWQKD
RNNRPKFEQIVSILDKLIRNPGSLKIITSAAARPSNLLLDQSNVDITT
FRTTGDWLNGVWTAHCKEIFTGVEYSSCDTIAKISTDDMKKVGVTVVG
PQKKIISSIKALETQSKNGPVPV

In this specification, “EphA3” refers to EphA3 from any species and includes EphA3 isoforms, fragments, variants (including mutants), or homologues from any species.

As used herein, a “fragment”, “variant”, or “homologue” of a protein may optionally be characterised as having at least 60%, preferably one of 70%, 75%, 80%, 85%, 90%, 91%, 92%, 93%, 94%, 95%, 96%, 97%, 98%, 99%, or 100% amino acid sequence identity to the amino acid sequence of the reference protein (e.g., a reference isoform). In some embodiments, fragments, variants, isoforms, and homologues of a reference protein may be characterised by ability to perform a function performed by the reference protein.

A “fragment” generally refers to a segment, domain, portion or region of a reference protein, which constitutes less than 100% of the amino acid sequence of the protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g., at least 60%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein. Homologues include orthologues.

A fragment may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein. A fragment of EphA3 may have a minimum length of one of 10, 20, 30, 40, 50, 60, 70, 80, 90, 100, 110, 120, 130, 140, 150, 200, 250, 300, 350, 400, 450, 550 or up to about 600 amino acids, and may have a maximum length of one of 20, 30, 40, 50, 60, 70, 80, 90, 100, 110, 120, 130, 140, 150, 200, 250, 300, 350, 400, 450, 550 or up to about 600 amino acids.

In some embodiments, the EphA3 is EphA3 from a mammal (e.g., a primate (rhesus, cynomolgus, non-human primate, or human) and/or rodent (e.g., rat or murine) EphA3). Isoforms, fragments, variants or homologues of EPhA3 may optionally be characterised as having at least 70%, preferably one of 80%, 85%, 90%, 91, 92, 93, 94, 95, 96, 97. 98. 99, or 100 amino acid sequence identity to the amino acid sequence of an immature or mature EphA3 isoform from a given species, e.g., human.

Isoforms, fragments, variants, or homologues may optionally be functional isoforms, fragments, variants, or homologues, e.g., having a functional property/activity of the reference EphA3, as determined by analysis by a suitable assay for the functional property/activity. For example, an isoform, fragment, variant, or homologues of EphA3 may e.g., display association with EphA5, or retain kinase activity.

In some embodiments, the EphA3 comprises, or consists of, an amino acid sequence having at least 70%, preferably one of 80%, 85%, 90, 91, 92, 93, 94, 95, 96, 97, 98, 99, or 100 amino acid sequence identity to SEQ ID NO: 165 or 166. In some embodiments, a fragment of EphA3 comprises, or consists of, an amino acid sequence having at least 70%, preferably one of 80, 85, 90, 91, 92, 93, 94, 95, 96, 97, 98, 99, or 100 amino acid sequence identity to one of SEQ ID Nos: 167, 170, 171, 172, or a combination thereof.

EphA3 is a member of the ephrin receptor subfamily of the protein tyrosine kinase family, and is known to be aberrantly expressed in a variety of human cancers including malignant melanoma, glioblastoma, lung and breast cancer. Increased expression of EphA3 can promote tumour cellular proliferation, angiogenesis, and invasion.

### Regions of Interest on the Target Molecule

The antigen-binding molecules of the present invention were specifically designed to target regions of EphA3 of particular interest. In a two-step approach, EphA3 regions to be targeted were selected following analysis for predicted antigenicity, function and safety. Antibodies specific for the target regions of EphA3 were then prepared using peptides corresponding to the target regions as immunogens to raise specific monoclonal antibodies, and subsequent screening to identify antibodies capable for binding to EphA3 in the native state. This approach provides control over the antibody epitope.

The antigen-binding molecules of the present invention may be defined by reference to the region of EphA3 which they bind to. The antigen-binding molecules of the present invention may bind to a particular region of interest of EphA3. In some embodiments the antigen-binding molecule may bind to a linear epitope of EphA3, consisting of a contiguous sequence of amino acids (i.e., an amino acid primary sequence). In some embodiments, the antigen-binding molecule may bind to a conformational epitope of EphA3, constating of a discontinuous sequence of amino acids of the amino acid sequence.

In some embodiments, the antigen-binding molecule binds to EphA3. In some embodiments, the antigen-binding molecule binds to the extracellular region of EphA3 (e.g., the region shown in SEQ ID NO: 167). In some embodiments, the antigen-binding molecule binds to the domain of Eph ligand-binding domain (e.g., the region shown in SEQ ID NO: 170). In some embodiments, the antigen-binding molecule binds to one or both of the fibronectin type 11 domains (e.g., the regions shown in SEQ ID NO: 171 and 172).

The region of a peptide/polypeptide to which an antibody binds can be determined by the skilled person using various methods well known in the art, including X-ray crystallography, any analysis of antibody antigen complexes, peptide scanning, mutagenesis mapping, hydrogen-deuterium exchange analysis by mass spectrometry, phage display, competition ELISA and proteolysis-based “protection” methods. Such methods are described, for example, in Gershoni et al., BioDrugs, 2007, 21(3): 145-156, which is hereby incorporated by reference in its entirety.

In some embodiments the antigen-binding molecule is capable of binding the same region of EphA3, or an overlapping region of EphA3, to the region of EphA3 which is bound by an antibody comprising the VH and VL sequences of one of antibody clones 3C3-1 or 2D4-1 described herein.

As used herein, by “isolated” is meant material, such as an EphA3 binding molecule, that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in recombinant, chemical synthetic, enriched, purified or partially purified form.

As used herein a “protein” is an amino acid polymer, wherein the amino acids may include D-amino acids, L-amino acids, natural and/or non-natural amino acids. As typically used herein, a “peptide” is a protein comprising no more than fifty (50) contiguous amino acids. As typically used herein, a “polypeptide” is a protein comprising more than fifty (50) contiguous amino acids. The term “protein” should also be understood to encompass protein-containing molecules such as glycoproteins and lipoproteins, although without limitation thereto.

In some embodiments, the antigen-binding molecule of the present invention is capable of binding to a polypeptide comprising, or consisting of, the amino acid sequence of one of SEQ ID NOs: 165, 166, 167, 170, 171, or 172.

The ability of an antigen-binding molecule to bind to a given peptide/polypeptide can be analyses by methods well known to the skilled person, including analysis by ELISA, immunoblot (e.g., western blot), immunoprecipitation, Surface Plasmon Resonance (SPR; see, e.g., Hearty et al., Methods Mol. Biol. (2012) 907: 411-442), or Bio-Layer Interferometry (see, e.g., Lad et al., (2015) J. Biomol. Screen 20(4): 498-507).

In embodiments where the antigen binding molecule is capable of binding to a peptide or polypeptide comprising a reference amino acid sequence, the peptide or polypeptide may comprise one or more additional amino acids at one or both ends of the reference amino acid sequence. In some embodiments the peptide/polypeptide comprises, for example, 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence.

In some embodiments, the additional amino acid(s) provided at one or both ends (i.e., the N-terminal and C-terminal ends) of the reference sequence correspond to the positions at the ends of the reference sequence in the context of the amino acid sequence of EphA3. By way of example, where the antigen-binding molecule is capable of binding to a peptide or polypeptide comprising the sequence of SEQ ID NO: #3 #, and an additional two amino acids at the C-terminal end of SEQ ID NO: #3 #, the additional two amino acids may be both be valine, corresponding to positions 542 and 543 of SEQ ID NO: 165.

In some embodiments the antigen-binding molecule is capable of binding to a peptide/polypeptide which is bound by an antibody comprising the VH and VL sequences of one of antibody clones 3C3-1 or 2D4-1 described herein.

### Antigen-Binding Molecules

The present invention provides antigen-binding molecules capable of binding to EphA3.

An “antigen-binding molecule” refers to a molecule which is capable of binding to a target antigen, and encompasses monoclonal antibodies, polyclonal antibodies, monospecific antibodies and multi-specific antibodies (e.g., bispecific antibodies), and antibody fragments, as long as they display binding to the relevant target molecule.

In particular embodiments, the EphA3 binding molecule described herein is an antibody or antibody fragment. As used herein, an “antibody” is or comprises an immunoglobulin protein. The term “immunoglobulin” includes any antigen-binding protein product of a mammalian immunoglobulin gene complex, including immunoglobulin isotypes IgA, IgD, IgM, IgG and IgE and antigen-binding fragments thereof. Included in the term “immunoglobulin” are immunoglobulins that are recombinant, chimeric or humanized or otherwise comprise altered or variant amino acid residues, sequences and/or glycosylation, whether naturally occurring or produced by human intervention (e.g., by recombinant DNA technology).

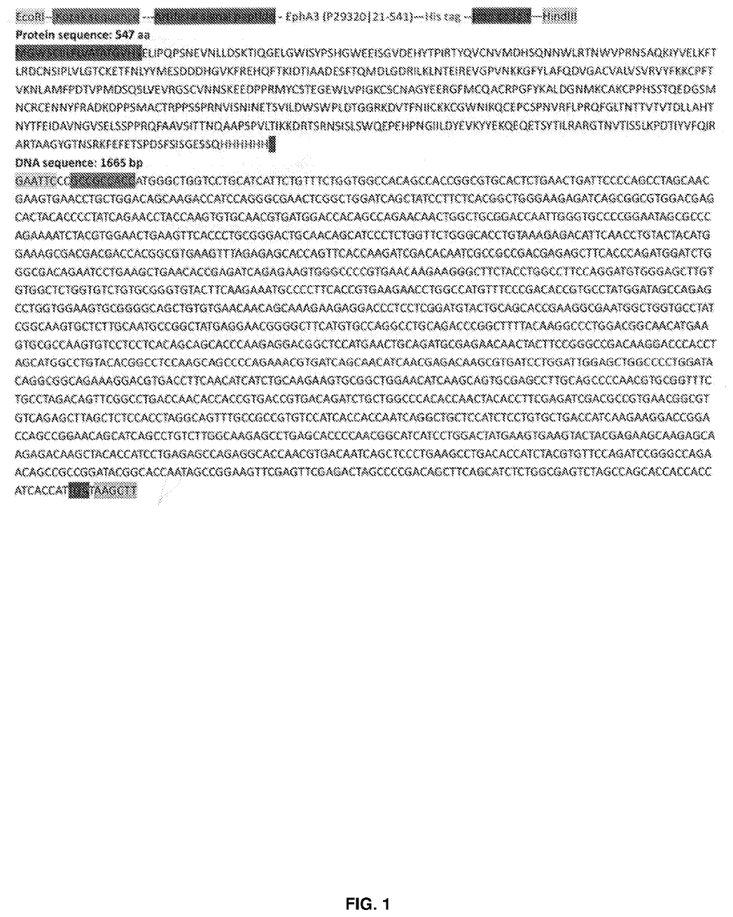

Generally, antibodies and antibody fragments may be polyclonal or monoclonal. In particular embodiments, the antibody or antibody fragment is one of those monoclonal antibodies provided in FIG. 1 (or a fragment thereof), such as an 3C3-1 or 2D4-1 monoclonal antibody or fragment thereof.

The invention also includes within its scope antibody fragments, such as Fv, Fc, Fab or F(ab′)₂ fragments of the polyclonal or monoclonal antibodies described herein. Alternatively, the EphA3 binding agents of the invention may comprise single chain Fv (scFvs) and/or scFab antibodies. Such scFvs may be prepared, for example, in accordance with the methods described respectively in U.S. Pat. No. 5,091,513, European Patent No 239,400 or the article by Winter & Milstein, 1991, Nature 349:293, which are incorporated herein by reference. The invention is also contemplated to include multivalent recombinant antibody fragments, so-called diabodies, triabodies and/or tetrabodies, comprising a plurality of scFvs, as well as dimerisation-activated demibodies (e.g., WO/2007/062466). By way of example, such antibodies may be prepared in accordance with the methods described in Holliger et al., 1993 Proc Natl Acad Sci USA 90:6444-6448; or in Kipriyanov, 2009 Methods Mol Biol 562:177-93 and herein incorporated by reference in their entirety.

It will also be appreciated that antibodies may be produced as recombinant synthetic antibodies or antibody fragments by expressing a nucleic acid encoding the antibody or antibody fragment in an appropriate host cell. Non-limiting examples of recombinant antibody expression and selection techniques are provided in Chapter 17 of Coligan et al., CURRENT PROTOCOLS IN IMMUNOLOGY and Zuberbuhler et al., 2009, Protein Engineering, Design & Selection 22 169.

Typically, an antibody comprises: respective light chain (VL or VL) and heavy chain (V_{H} or VH) variable regions that each comprise complementarity determining region (CDR) 1, 2 and 3 amino acid sequences; and respective light chain (CL) and heavy chain (CH₁, CH₂, CH₃) constant regions. Accordingly, antibodies generally comprise six CDRs (three in the heavy chain variable region), and three in the light chain variable region). The six CDRs together define the paratope of the antibody, which is the part of the antibody that binds to the target antigen.

The antigen-binding molecules of the present invention may be designed and prepared using the sequences of monoclonal antibodies (mAbs) capable of binding to EphA3. Antigen-binding regions of antibodies, such as single chain variable fragment (scFv), Fab and F(ab′)₂ fragments may also be used/provided. An “antigen-binding region” is any fragment of an antibody which is capable of binding to the target for which the given antibody is specific.

The V_{H} region and V_{L} region comprise framework regions (FRs) either side of each CDR, which provide a scaffold for the CDRs. From N-terminus to C-terminus, V_{H} regions comprise the following structure: N term-[HC-FR1]-[HC-CDR1]-[HC-FR2]-[HC-CDR2]-[HC-FR3]-[HC-CDR3]-[HC-FR4]-C term; and V_{L} regions comprise the following structure: N term-[LC-FR1]-[LC-CDR1]-[LC-FR2]-[LC-CDR2]-[LC-FR3]-[LC-CDR3]-[LC-FR4]-C term.

CDR identification and numbering may be according to any known CDR numbering system inclusive of Kabat (Kabat et al., Sequences of Proteins of Immunological Interest, 5th Ed. Public Health Service, National Institutes of Health, Bethesda, MD (1991)), Chothia (Chothia et al., J. Mol. Biol. 196:901-917 (1987)), AbM and Contact.

In some embodiments, the antigen-binding molecule comprises the CDRs of an antigen-binding molecule which is capable of binding to EphA3. In some embodiments, the antigen-binding molecule comprises the FRs of an antigen-binding molecule which is capable of binding to EphA3. In some embodiments, the antigen-binding molecule comprises the CDRs and the FRs of an antigen-binding molecule which is capable of binding to EphA3. That is, in some embodiments the antigen-binding molecule comprises the V_{H} region and the V_{L} region of an antigen-binding molecule which is capable of binding to EphA3.

In some embodiments the antigen-binding molecule comprises a V_{H} and a V_{L} region which is, or which is derived from, the V_{H}/V_{L} region of an EphA3-binding antibody close described herein (i.e., anti-EphA3 antibody clones 3C3-1 or 2D4-1). Non-limiting examples of CDR amino acid sequences are set forth in SEQ ID NOS: 13-72 and/or Tables 2-5. CDR identification and numbering was performed using abYsis version 3.4.1 and IMGT/V-QUEST. Antibodies according to the invention may comprise 1, 2 or 3 V_{L} CDR amino acid sequences (e.g., CDR1, CDR2 and/or CDR3) and/or 1, 2, or 3 V_{H} CDR amino acid sequences (e.g., CDR1, CDR2 and/or CDR3), such as those set forth in SEQ ID NOS: 13-72 and/or Tables 2-5.

In some embodiments, the EphA3 binding agent comprises:
(a) a heavy chain immunoglobulin variable region (V_{H}) polypeptide comprising a CDR1 having an amino acid sequence at least 70% identical to any one of SEQ ID NOs:13-17; a CDR2 having an amino acid sequence at least 70% identical to any one of SEQ ID NOs: 18 to 22; and a CDR3 having an amino acid sequence at least 70% identical to any one of SEQ ID NO: 23 to 27; and/or
(b) a light chain immunoglobulin variable region (V_{L}) polypeptide comprising a CDR1 having an amino acid sequence at least 70% identical to any one of SEQ ID NOs: 28-32; a CDR2 having an amino acid sequence at least 70% identical to any one of SEQ ID NOs: 33-37; and a CDR3 having an amino acid sequence at least 70% identical to any one of SEQ ID NOs: 38-42.

With regard to such embodiments, the V_{H} polypeptide suitably comprises an amino acid sequence set forth in SEQ ID NO:153 or an amino acid sequence at least 70%, 75%, 80%, 85%, 90%, 95%, 96%, 97%, 98%, 99%, or 100% identical thereto; and/or the V_{L} polypeptide suitably comprises an amino acid sequence set forth in SEQ ID NO:154 or an amino acid sequence at least 70%, 75%, 80%, 85%, 90%, 95%, 96%, 97%, 98%, 99%, or 100% identical thereto.

In an alternative embodiment, the EphA3 binding agent comprises:
(a) a V_{H} polypeptide comprising a CDR1 having an amino acid sequence at least 70% identical to any one of SEQ ID NOs: 43-47; a CDR2 having an amino acid sequence at least 70% identical to any one of SEQ ID NOs: 48-52; and a CDR3 having an amino acid sequence at least 70% identical to any one of SEQ ID NOs: 53-57; and/or
(b) a V_{L} polypeptide comprising a CDR1 having an amino acid sequence at least 70% identical to any one of SEQ ID NOs: 58-62; a CDR2 having an amino acid sequence at least 70% identical to any one of SEQ ID NOs: 63-67; and a CDR3 having an amino acid sequence at least 70% identical to any one of SEQ ID NOs: 68-72.

In this regard, the V_{H} polypeptide can comprise an amino acid sequence set forth in SEQ ID NO: 155 or an amino acid sequence at least 70% identical thereto; and/or the V_{L} polypeptide can comprise an amino acid sequence set forth in SEQ ID NO: 156 or an amino acid sequence at least 70% identical thereto.

The CDRs and FRs of the V_{H} regions and V_{L} regions of the antibody closes described herein are below defined according to the international IMGT (ImMunoGeneTics) information system (LeFranc et al., Nucleic Acids Res., (2015) 43 (Database issue): D413-22), which uses the IMGT V-DOMAIN numbering rules as described in LeFranc et al., Dev. Comp. Immunol. (2003) 27: 55-77. In some embodiments, the antigen-binding molecule comprises a V_{H} region according to (1) or (2) below:
(1) (3C3-1) a V_{H} region incorporating the following CDRs:
   HC-CDR1 having the amino acid sequence of SEQ ID NO: 16;
   HC-CDR2 having the amino acid sequence of SEQ ID NO: 22;
   HC-CDR3 having the amino acid sequence of SEQ ID NO: 27;
      or a variant thereof in which one or two or three amino acids in one or more of HC-CDR2, HC-CDR2, or HC-CDR3 are substituted with another amino acid.
(2) (2D4-1) a V_{H} region incorporating the following CDRs:
   HC-CDR1 having the amino acid sequence of SEQ ID NO: 47;
   HC-CDR2 having the amino acid sequence of SEQ ID NO: 52;
   HC-CDR3 having the amino acid sequence of SEQ ID NO: 57;
      or a variant thereof in which one or two or three amino acids in one or more of HC-CDR2, HC-CDR2, or HC-CDR3 are substituted with another amino acid.

In some embodiments, the antigen-binding molecule comprises a V_{H} region according to (3) or (4), below:
(3) (3C3-1) a V_{H} region incorporating the following FRs:
   HC-FR1 having the amino acid sequence of SEQ ID NO: 97;
   HC-FR2 having the amino acid sequence of SEQ ID NO: 102;
   HC-FR3 having the amino acid sequence of SEQ ID NO: 107;
   HC-FR4 having the amino acid sequence of SEQ ID NO: 112;
   or a variant thereof in which one or two or three amino acids in one or more of HC-FR1, HC-FR2, HC-FR3, or HC-FR4 are substituted with another amino acid.
(4) (2D4-1) a V_{H} region incorporating the following FRs:
   HC-FR1 having the amino acid sequence of SEQ ID NO: 137;
   HC-FR2 having the amino acid sequence of SEQ ID NO: 142;
   HC-FR3 having the amino acid sequence of SEQ ID NO: 147;
   HC-FR4 having the amino acid sequence of SEQ ID NO: 152;
   or a variant thereof in which one or two or three amino acids in one or more of HC-FR1, HC-FR2, HC-FR3, or HC-FR4 are substituted with another amino acid.

In some embodiments the antigen-binding molecule comprises a V_{H} region comprising the CDRs according to one of (1) and (2) above, and the FRs according to (3) or (4) above.

In some embodiments, the antigen-binding molecule comprises a V_{H} region according to one of (5) or (6) below:
(5) a V_{H} region comprising the CDRs according to (1) and the FRs according to (3).
(6) A V_{H} region comprising the CDRs according to (2) and the FRs according to (4).

In some embodiments, the antigen-binding molecule comprises a V_{L} region according to (7) or (8) below:
(7) (3C3-1) a V_{L} region incorporating the following CDRs:
   LC-CDR1 having the amino acid sequence of SEQ ID NO: 32;
   LC-CDR2 having the amino acid sequence of SEQ ID NO: 37;
   LC-CDR3 having the amino acid sequence of SEQ ID NO: 42;
      or a variant thereof in which one or two or three amino acids in one or more of LC-CDR2, LC-CDR2, or LC-CDR3 are substituted with another amino acid.
(8) (2D4-1) a V_{L} region incorporating the following CDRs:
   LC-CDR1 having the amino acid sequence of SEQ ID NO: 62;
   LC-CDR2 having the amino acid sequence of SEQ ID NO: 67;
   LC-CDR3 having the amino acid sequence of SEQ ID NO: 72;
      or a variant thereof in which one or two or three amino acids in one or more of LC-CDR2, LC-CDR2, or LC-CDR3 are substituted with another amino acid.

In some embodiments, the antigen-binding molecule comprises a V_{L} region according to (9) or (10), below:
(9) (3C3-1) a V_{L} region incorporating the following FRs:
   LC-FR1 having the amino acid sequence of SEQ ID NO: 97;
   LC-FR2 having the amino acid sequence of SEQ ID NO: 102;
   LC-FR3 having the amino acid sequence of SEQ ID NO: 107;
   LC-FR4 having the amino acid sequence of SEQ ID NO: 112;
   or a variant thereof in which one or two or three amino acids in one or more of LC-FR1, LC-FR2, LC-FR3, or LC-FR4 are substituted with another amino acid.
(10) (2D4-1) a V_{L} region incorporating the following FRs:
   LC-FR1 having the amino acid sequence of SEQ ID NO: 137;
   LC-FR2 having the amino acid sequence of SEQ ID NO: 142;
   LC-FR3 having the amino acid sequence of SEQ ID NO: 147;
   LC-FR4 having the amino acid sequence of SEQ ID NO: 152;
   or a variant thereof in which one or two or three amino acids in one or more of LC-FR1, LC-FR2, LC-FR3, or LC-FR4 are substituted with another amino acid.

In some embodiments the antigen-binding molecule comprises a V_{L} region comprising the CDRs according to one of (1) and (2) above, and the FRs according to (3) or (4) above.

In some embodiments, the antigen-binding molecule comprises a V_{H} region according to one of (11) or (12) below:
(11) a V_{H} region comprising the CDRs according to (7) and the FRs according to (9).
(12) A V_{H} region comprising the CDRs according to (8) and the FRs according to (10).

The V_{H} and V_{L} region of an antigen-binding region of an antibody together constitute the Fv region. In some embodiments, the antigen-binding molecule according to the present invention comprises or consists of, an Fv region which binds to EphA3. In some embodiments, the V_{H} and V_{L} regions of the Fv are provided as a single polypeptide joined by a linker region, i.e., a single chain Fv (scFv).

In some embodiments, the invention provides fragments of the isolated antibodies and the CARs of the invention.

Fragments of the invention can be produced by those methods described herein. Alternatively, fragments can be produced, for example, by digestion of an antibody or CAR protein with proteinases such as endoLys-C, endoArg-C, endoGlu-C and V8-protease. The digested fragments can be purified by chromatographic techniques as are well known in the art.

Particular embodiments of the invention provide an immunogenic fragment of the EphA3 antigen-binding molecules of the invention. By “immunogenic” is meant capable of eliciting an immune response upon administration to an animal, such as a human, mouse or rabbit. The immune response may include the production, activation or stimulation of the innate and/or adaptive arms of the immune system inclusive of immune cells such as B and/or T lymphocytes, NK cells, granulocytes, macrophages and dendritic cells and/or molecules such as antibodies, cytokines and chemokines, although without limitation thereto.

Antibody fragments include Fab and Fab′2 fragments, diabodies, triabodies, bispecific antibodies and single chain antibody fragments (e.g., ScFvs), although without limitation thereto. In some embodiments, an antibody fragment may comprise at least a portion of a CDR1, 2 and/or 3 amino acid sequence, such as set forth in SEQ ID NOS:13-72 or a V_{H} and/or V_{L} amino acid sequence, such as set forth in SEQ ID NOS:153-156. A preferred antibody fragment comprises at least one entire light chain variable region CDR and/or at least one entire heavy chain variable region CDR.

In some embodiments, the EphA3 binding agent provided herein is a recombinant, human or humanized antibody or antibody fragment. As broadly used herein, “humanized” antibodies may include antibodies entirely or at least partly of human origin, inclusive of modified antibodies or antibody fragments obtained from a non-human “foreign” species. In some embodiments, antibodies and antibody fragments may be modified so as to be administrable to one species having being produced in, or originating from, the same or another “foreign” species without eliciting a deleterious immune response to the “foreign” antibody. Human or non-human antibody fragments such as comprising complementarity determining regions (CDRs) or variable regions (i.e., V_{H} and V_{L} domains) may be “grafted” onto a human antibody scaffold or backbone to produce a “humanized” antibody or antibody fragment. In some embodiments, human or non-human CDRs or V_{L} and V_{L} domains are recombinantly grafted with a human antibody constant region

In some embodiments, the antigen-binding molecule of the present invention comprises one or more regions of an immunoglobulin heavy chain constant sequence. In some embodiments, the immunoglobulin heavy chain constant sequence is, or is derived from, the heavy chain constant sequence of an IgG (e.g., IgG1, IgG2, IgG3, IgG4), IgA (e.g., IgA1, IgA2), IgD, IgE, or IgM.

In some embodiments, the immunoglobulin heavy chain constant sequence is human immunoglobulin G 1 constant (IGHG1: UniProt accession no. P01857, v1; SEQ ID NO: 175). Positions 1 to 98 of SEQ ID NO: 175 form the CH1 region (SEQ ID NO: 176). Positions 99 to 110 of SEQ ID NO: 175 form a hinge region between CH1 and CH2 regions (SEQ ID NO: 177). Positions 111 to 223 of SEQ ID NO: 175 form the CH2 region (SEQ ID NO: 178). Positions 224 to 330 of SEQ ID NO: 175 form the CH3 region (SEQ ID NO: 179).

Immunoglobulin heavy chain constant gamma 1
[SEQ ID NO: 175]
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
K

In some embodiments, a CH1 region comprises or consists of the sequence SEQ ID NO: 176, or a sequence having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of SEQ ID NO: 176.

In some embodiments, the antigen-binding molecule of the present invention comprises one or more regions of an immunoglobulin light chain constant sequence. In some embodiments, the immunoglobulin light chain constant sequence is a human immunoglobulin lambda constant sequence (IGLA; CA), e.g., IGLC1, IGLC2, IGLC3, IGLC6, or IGLC7. In some embodiments a CL region comprises or consists of the sequence of SEQ ID NO: 180, or a sequence having at least 60%, preferably one of 70%, 75%, 80%, 85%, 90%, 91%, 92%, 93%, 94%, 95%, 96%, 97%, 98%, 99%, or 100% amino acid sequence identity to the amino acid sequence of SEQ ID NO: 180.

Immunoglobulin lambda constant region
[SEQ ID NO: 180]
MRPGTGQGGLEAPGEPGPNLRQRWPLLLLGLAVVTHGLLRPTAASQS
RALGPGAPGGSSRSSLRSRWGRFLLQRGSWTGPRCWPRGFQSKHNSV
THVFGSGTQLTVLSQPKATPSVTLFPPSSEELQANKATLVCLMNDFY
PGILTVTWKADGTPITQGVEMTTPSKQSNNKYAASSYLSLTPEQWRS
RRSYSCQVMHEGSTVEKTVAPAECS
Immunoglobulin kappa constant region
[SEQ ID NO: 212]
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGL
SSPVTKSFNRGEC

The V_{L} and light chain constant (CL) region, and the V_{H} region and heavy chain constant 1 (CH1) region of an antigen-binding region of an antibody together constitute the Fab region. In some embodiments, the antigen-binding molecule comprises a Fab region comprising a V_{H}, a CH1, a V_{L} and a CL (e.g., CK or CA). In some embodiments, the Fab region comprising a V_{H} and a CH1 (e.g., a VH-CH1 fusion polypeptide). In some embodiments, the Fab region comprises a polypeptide comprising a V_{H} and a CL (e.g., a V_{H}-CL fusion polypeptide). In some embodiments, the Fab region comprises a polypeptide comprising a V_{H} and a CL (e.g., a V_{H}-CL fusion polypeptide) and a polypeptide comprising a V_{L} and a CH (e.g., a CL-CH1 fusion polypeptide; that is, in some embodiments the Fab region is a CrossFab region. In some embodiments, the V_{H}, CH1, V_{L}, and CL regions of the Fab or CrossFab are provided as a single polypeptide joined by linker regions, i.e., as a single chain Fab (scFab) or a single chain CrossFab (scCrossFab).

In some embodiments, the antigen-binding molecule of the present invention comprises, consists, or consists essentially of, a Fab region which binds to EphA3. In some embodiments, the antigen-binding molecule described herein comprises, or consists of, a whole antibody which binds to EphA3. As used herein “whole antibody” refers to an antibody having a structure which is substantially similar to the structure of an immunoglobulin (Ig). Different kinds of immunoglobulins and their structures are described, for example, in Schroeder and Cavacini, J Allergy Clin Immunol (2010) 125(202): S41-S52, which is hereby incorporated by reference in its entirety.

Immunoglobulins of type G (i.e., IgG) are about 150 kDa glycoproteins comprising two heavy chains and two light chains. From N- to C-terminus, the heavy chains comprise a V_{H} followed by a heavy chain constant region comprising three constant domains (CH1, CH3, and CH3), and similarly the light chain comprise a V_{L} followed by a CL. Depending on the heavy chain, immunoglobulins may be classed as IgG (e.g., IgG1, IgG2, IgG3, IgG4), IgA (e.g., IgA1, IgA2), IgD, IgE, or IgM. The light chain may be kappa (κ) or lambda (A).

In some embodiments, the antigen-binding molecule describe herein comprises, consists, or consists essentially of an IgG e.g., IgG1, IgG2, IgG3, IgG4), IgA (e.g., IgA1, IgA2), IgD, IgE, or IgM which binds to EphA3.

Suitably, the EphA3 binding agent binds an epitope of an EphA3 protein. As generally used herein, an “epitope” is an antigenic protein fragment that comprises a continuous or discontinuous sequence of amino acids of a protein, wherein the epitope can be recognized or bound by an element of the immune system, such as an antibody or other antigen receptor.

The invention also includes variants of the EphA3 binding agent disclosed herein. In one embodiment, the variant is an EphA3 binding agent comprising an amino acid sequence at least 70% identical to any one of SEQ ID NOS:13-72, referred to herein as a CDR “variant”. In another embodiment, the variant comprises an amino acid sequence at least 70% identical to the V_{H} and/or V_{L} amino acid sequence of any one of SEQ ID NOS: 153-156.

Suitably, an EphA3 binding agent comprising at least one of the CDR or other variant(s) is capable of binding an EphA3 protein.

In particular embodiments, a variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% amino acid sequence identity to the amino acid sequence of the reference protein (e.g., a reference isoform), such as those set forth in any one of SEQ ID NOS:13-156. The protein “variant” disclosed herein may have one or more amino acids deleted, inserted, or substituted by different amino acids. It is well understood in the art that some amino acids may be substituted or deleted without changing biological activity of the peptide (conservative substitutions). In some embodiments, fragments, variants, isoforms and homologues or a reference protein may be characterised by ability to perform a function performed by the reference protein.

Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., lie, Thr, and Val), an amino acid with an aromatic side-chain substituted for another amino acid with an aromatic side chain (e.g., His, Phe, Trp, and Tyr), etc.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-2015).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

Derivatives of the antibody, antibody fragments or variants thereof disclosed herein are also provided.

As used herein, “derivative” antibodies, antibody fragments or variants thereof have been altered, for example by conjugation or complexing with other chemical moieties, by post-translational modification (e.g. phosphorylation, ubiquitination, glycosylation), chemical modification (e.g. cross-linking, acetylation, biotinylation, oxidation or reduction and the like), conjugation with labels (e.g. fluorophores, enzymes, radioactive isotopes) and/or inclusion of additional amino acid sequences as would be understood in the art.

In this regard, the skilled person is referred to Chapter 15 of CURRENT PROTOCOLS IN PROTEIN SCIENCE, Eds. Coligan et al. (John Wiley & Sons NY 1995-2015) for more extensive methodology relating to chemical modification of proteins.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP, RFP), epitope tags such as myc, FLAG and haemagglutinin tags.

The isolated proteins (e.g., EphA3 antibodies, antibody fragments and CARs), variants, fragments and/or derivatives of the present invention may be produced by any means known in the art, including but not limited to, chemical synthesis, recombinant DNA technology and proteolytic cleavage to produce peptide fragments.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444.

In one preferred embodiment, the EphA3 antibodies, antibody fragments and/or CAR proteins of the present invention are recombinant proteins.

Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6.

### Chimeric Antigen Receptors (CARs)

The present invention also provides Chimeric Antigen Receptors (CARs) comprising the antigen-binding molecules or polypeptides of the present invention.

Therefore, in a related aspect of the invention provides a chimeric antigen receptor (CAR) comprising an antigen binding domain including at least one CDR having an amino acid sequence set forth in SEQ ID NOs:13-72 or an amino acid sequence at least 70% identical thereto, a transmembrane domain, and an intracellular T cell signalling domain.

A CAR is an artificially constructed hybrid protein or polypeptide containing the antigen binding domains of an antibody (e.g., single chain variable fragment (scFv)) linked to a T-cell signalling domain. Characteristics of CARs include their ability to redirect T-cell specificity and reactivity toward a selected target in a non-MHC-restricted manner and exploiting the antigen-binding properties of monoclonal antibodies. The non-MHC-restricted antigen recognition gives T-cells expressing CARs the ability to recognize antigens independent of antigen processing, thus bypassing a major mechanism of tumour escape. Moreover, when expressed in T-cells, CARs advantageously do not dimerize with endogenous T cell receptor (TCR) alpha and beta chains. CAR structure and engineering is reviewed, for example, in Dotti et al, Immunol Rev (2014) 257(1), hereby incorporated by reference in its entirety. CARs comprise an antigen-binding region linked to a cell membrane anchor region (also known as the transmembrane domain) and a signalling region. An optional hinge region may provide separation between the antigen-binding region and cell membrane anchor region, and may act as a flexible linker. The CAR of the present invention comprises an antigen-binding region which comprises, consists, or consists essentially of polypeptide according to the invention.

The cell membrane anchor region is provided between the antigen-binding domain and the signalling region of the CAR and provides for anchoring the CAR to the cell membrane of a cell expressing a CAR, with the antigen-binding region in the extracellular space, and signalling region inside the cell. In some embodiments, the CAR comprises of, or is derived from, the transmembrane region amino acid sequence for one of CD3-ζ, CD4, CD8, or CD28. Suitably, the transmembrane domain is derived from a membrane protein selected from CD8a, CD8p, 4-1 BB/CD137, CD28, CD34, CD4, FcεRIγ, CD16, OX40/CD134, CD3-ζ, CD3ε, CD3γ, CD3δ, TCRα, CD32, CD64, VEGFR2, FAS, FGFR2B and any combination thereof. In some particular embodiments, the transmembrane domain may be derived from a CD8 and/or CD28 transmembrane domain, which generally provide good receptor stability. As used herein, a region which is “derived from” a reference amino acid sequence comprises an amino acid sequence having at least amino acid sequence at least 70%, 75%, 80%, 85%, 90%, 91%, 92%, 93%, 94%, 95%, 96%, 97%, 98%, 99%, or 100% sequence identical to the reference sequence. In some embodiments, the transmembrane domain comprises an amino acid sequence set forth in SEQ ID NO:159 or an amino acid sequence at least 70%, 75%, 80%, 85%, 90%, 91%, 92%, 93%, 94%, 95%, 96%, 97%, 98%, 99%, or 100% sequence identical thereto.

The transmembrane domain (i.e., the cell membrane anchor region) of the chimeric receptors described herein can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, it can be a synthetic, non-naturally occurring protein segment (e.g., a hydrophobic protein segment that is thermodynamically stable in a cell membrane; see e.g., U.S. Pat. No. 7,052,906 and PCT Publication No. WO 2000/032776, which are incorporated by reference herein). To this end, the transmembrane domain may comprise a hydrophobic alpha helix.

Any intracellular or cytoplasmic T-cell signalling domain (e.g., CD3-ζ or FcεR1γ) can be used to construct the chimeric receptors described herein, such as those comprising an immunoreceptor tyrosine-based activation motif (ITAM), for phosphorylation and activation of the CAR-expressing T-cell. An “ITAM,” as used herein, is a conserved protein motif that is generally present in the tail portion of signalling molecules expressed in many immune cells. After antigen recognition, receptors cluster and a signal is transmitted to the cell. The most commonly used T-cell signalling component is that of CD3-ζ which contains three ITAMs. This transmits an activation signal to the T-cell after antigen is bound. It will be appreciated, however, that the CD3-ζ cytoplasmic signalling domain may not provide a fully competent activation signal and an additional co-stimulatory signalling domain, such as those hereinbefore described may be utilised. For example, chimeric CD28 and/or 4-1 BB/CD137 can be used with CD3-ζ to transmit a proliferative/survival signal, or all three can be used together. Accordingly, the endodomain of the CAR of the invention may comprise a CD28 co-stimulatory domain (e.g., SEQ ID NO: 161), a 4-1 BB/CD137 co-stimulatory domain (e.g., SEQ ID NO: 160) and a CD3-ζ intracellular signalling domain (e.g., SEQ ID NO: 162). Signalling regions of CARs may also comprise co-stimulatory sequences derived from the signalling region of co-stimulatory molecules, to facilitate activation of CAR-expressing T-cells upon binding to the target protein. Activation of a co-stimulatory signalling domain in a host cell (e.g., an immune cell) may induce the cell to increase or decrease the production and secretion of cytokines, phagocytic properties, proliferation, differentiation, survival, and/or cytotoxicity. The co-stimulatory signalling domain of any co-stimulatory molecule may be compatible for use in the chimeric receptors described herein. The type(s) of co-stimulatory signalling domain is selected can be based on factors such as the type of the immune cells in which the chimeric receptors would be expressed (e.g., T cells, NK cells, macrophages, neutrophils, or eosinophils) and the desired immune effector function (e.g., ADCC effect). In other words, the term “co-stimulatory signalling domain”, as used herein, refers to at least a portion of a protein that mediates signal transduction within a cell to induce an immune response, such as an effector function. The co-stimulatory signalling domain of the chimeric receptor described herein can be a cytoplasmic signalling domain from a co-stimulatory protein, which transduces a signal and modulates responses mediated by immune cells, such as T cells, NK cells, macrophages, neutrophils, or eosinophils

Exemplary co-stimulatory signalling domains for use in the chimeric receptors can be the cytoplasmic signalling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF-R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-β, OX40/TNFRSF4, OX40 Ligand/TNFSF4, RELT/TNFRSF19L, TACI/TNFRSF13B, TL1A/TNFSF15, TNF, and TNF RII/TNFRSF1 B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA class I, HLA-DR, Ikaros, integrin α4/CD49d, integrin α4β1, integrin α4β7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signalling domain is of 4-1BB, CD28, OX40, ICOS, CD27, GITR, HVEM, TIM1, LFA1(CD11 a) or CD2, or any variant thereof. In some embodiments, the co-stimulatory signalling domain is derived from 4-1BB (e.g., SEQ ID NO: 160) and/or CD28 (e.g., SEQ ID NO: 161).

Also within the scope of the present disclosure are variants of any of the co-stimulatory signalling domains described herein, such that the co-stimulatory signalling domain is capable of modulating the immune response of the immune cell. Additionally, it is envisaged that the chimeric receptors may comprise more than one co-stimulatory signalling domain (e.g., 2, 3, 4 or more). In some embodiments, the chimeric receptor comprises two or more of the same co-stimulatory signalling domains, for example, two copies of the co-stimulatory signalling domain of CD28. In some embodiments, the chimeric receptor comprises two or more co-stimulatory signalling domains from different co-stimulatory proteins, such as any two or more co-stimulatory proteins described herein. In some cases, CARs are engineered to provide for co-stimulation of different intracellular signalling pathways. For example, signalling associated with CD28 co-stimulation preferentially activates the phosphatidylinositol 3-kinase (P13K) pathway, whereas the 4-1 BB-mediated signalling is through TNG receptor associated factor (TRAF) adaptor proteins. Signalling regions of CARs therefore sometimes contain co-stimulatory sequences derived from signalling regions of more than one co-stimulatory molecule. In some embodiments, the CAR of the present invention comprises one or more co-stimulatory sequences comprising or consisting of an amino acid sequences which comprises, consists of, or is derived from amino acid sequence of the intracellular domain of one or more of CD28, OX30, 4-1BB, ICOS, and CD27

An optional hinge region may provide separation between the antigen-binding domain and the transmembrane domain, and may act as a flexible linker. Hinge regions may be derived from IgG1. In some embodiments, the CAR of the present invention comprises a hinge region comprising or consisting of an amino acid sequence which comprises, consists of, or is derived from, the amino acid sequence of the hinge region of IgG1.

It is envisaged that the CARs of the invention may be considered to be, for example, a first generation, second generation, third generation or fourth generation (i.e., associated with a T-cell redirected for universal cytokine-mediated killing (TRUCKs)) CAR, as are known in the art. First generation CARs typically join an antibody-derived scFv to the CD3-zeta ((or z) intracellular signalling domain of the T-cell receptor through hinge and transmembrane domains. Second generation CARs incorporate an additional domain (e.g., CD28, 4-1BB, or ICOS) to supply a costimulatory signal. Third-generation CARs typically contain two costimulatory domains fused with the TCR CD3-ζ chain. Third-generation costimulatory domains may include, for example, a combination of CD3-ζ, CD27, CD28, 4-1BB, ICOS, DAP-10 or OX40. Accordingly, the CARs of the invention may contain an ectodomain commonly derived from a single chain variable fragment (scFv), a hinge, a transmembrane domain, and an endodomain with one (first generation), two (second generation), or three (third generation) signalling domains derived from CD3-ζ and/or co-stimulatory molecules.

In some embodiments, the CAR is associated with a T-cell redirected for cytokine activity (e.g., TRUCK), also known as a fourth generation CAR. TRUCKs are CAR-redirected T-cells used as vehicles to trigger effector activity of the CAR T cells and in addition produce and release a transgenic cytokine (e.g., IL-12) that accumulates in the targeted tissue (e.g., a tumour tissue that expresses EphA3). The transgenic cytokine is made constitutively or released upon CAR engagement of the target. TRUCK cells may deposit a variety of therapeutic cytokines at the target site. This may result in therapeutic concentrations at the targeted site and avoid systemic toxicity of these same cytokines.

The CARs of the invention suitably have antigen specificity for EphA3. The phrases “have antigen specificity” and “elicit antigen-specific response” as used herein means that the CAR can specifically bind to and immunologically recognize an antigen, such that binding of the CAR to the antigen elicits an immune response. Without being bound to a particular theory or mechanism, it is believed that by eliciting an antigen-specific response against EphA3, the CARs described herein provide for one or more of any of the following: targeting and destroying EphA3-expressing cancer cells, reducing or eliminating cancer cells, facilitating infiltration of immune cells to tumour site(s), and enhancing/extending anti-cancer responses.

An embodiment of the invention provides a CAR comprising an antigen binding domain of one of the monoclonal antibodies described herein, such as those provided in FIG. 1. In particular embodiments, the CAR comprises an antigen binding domain of the 3C3 or 2D4 monoclonal antibodies, which specifically bind to EphA3. In this regard, a preferred embodiment of the invention provides CARs comprising an antigen-binding domain comprising, consisting of, or consisting essentially of, a single chain variable fragment (scFv) of the antigen binding domain of 3C3 or 2D4.

The antigen binding domain may comprise a light chain variable region and/or a heavy chain variable region. In an embodiment of the invention, the heavy chain variable region comprises a CDR1 region, a CDR2 region, and a CDR3 region. In this regard, the antigen binding domain may comprise one or more of a heavy chain CDR1 region comprising any one of SEQ ID NOs: 13-17 or an amino acid sequence at least 70%, 75%, 80%, 85%, 90%, 91%, 92%, 93%, 94%, 95%, 96%, 97%, 98%, 99%, or 100% identical thereto; a heavy chain CDR2 region comprising any one of SEQ ID NOs: 18-22 or an amino acid sequence at least 70%, 75%, 80%, 85%, 90%, 91%, 92%, 93%, 94%, 95%, 96%, 97%, 98%, 99%, or 100% identical thereto; and a heavy chain CDR3 region comprising any one of SEQ ID NOs: 23-27 or an amino acid sequence at least 70%, 75%, 80%, 85%, 90%, 91%, 92%, 93%, 94%, 95%, 96%, 97%, 98%, 99%, or 100% identical thereto. In an alternative embodiment, the antigen binding domain comprises one or more of a heavy chain CDR1 region comprising any one of SEQ ID NOs: 43-47 or an amino acid sequence at least 70%, 75%, 80%, 85%, 90%, 91%, 92%, 93%, 94%, 95%, 96%, 97%, 98%, 99%, or 100% identical thereto; a heavy chain CDR2 region comprising any one of SEQ ID NOs: 48-52 or an amino acid sequence at least 70%, 75%, 80%, 85%, 90%, 91%, 92%, 93%, 94%, 95%, 96%, 97%, 98%, 99%, or 100% identical thereto; and a heavy chain CDR3 region comprising any one of SEQ ID NOs: 53-57 or an amino acid sequence at least 70%, 75%, 80%, 85%, 90%, 91%, 92%, 93%, 94%, 95%, 96%, 97%, 98%, 99%, or 100% identical thereto. Preferably, the heavy chain comprises all of a CDR1 region, a CDR2 region, and a CDR3 region selected from SEQ ID NOs: 13-27 or SEQ ID NOs: 43-57 or an amino acid sequence at least 70%, 75%, 80%, 85%, 90%, 91%, 92%, 93%, 94%, 95%, 96%, 97%, 98%, 99%, or 100% identical thereto.

In an embodiment of the invention, the light chain variable region may comprise a light chain CDR1 region, a light chain CDR2 region, and a light chain CDR3 region. In this regard, the antigen binding domain may comprise one or more of a light chain CDR1 region comprising any one of SEQ ID NOs: 28-32 or an amino acid sequence at least 70%, 75%, 80%, 85%, 90%, 91%, 92%, 93%, 94%, 95%, 96%, 97%, 98%, 99%, or 100% identical thereto; a light chain CDR2 region comprising any one of SEQ ID NOs: 33-37 or an amino acid sequence at least 70%, 75%, 80%, 85%, 90%, 91%, 92%, 93%, 94%, 95%, 96%, 97%, 98%, 99%, or 100% identical thereto; and a light chain CDR3 region comprising any one of SEQ ID NOs: 38-42 or an amino acid sequence at least 70%, 75%, 80%, 85%, 90%, 91%, 92%, 93%, 94%, 95%, 96%, 97%, 98%, 99%, or 100% identical thereto. In an alternative embodiment, the antigen binding domain comprises one or more of a light chain CDR1 region comprising any one of SEQ ID NO: 58-62 or an amino acid sequence at least 70%, 75%, 80%, 85%, 90%, 91%, 92%, 93%, 94%, 95%, 96%, 97%, 98%, 99%, or 100% identical thereto; a light chain CDR2 region comprising any one of SEQ ID NO: 63-67 or an amino acid sequence at least 70%, 75%, 80%, 85%, 90%, 91%, 92%, 93%, 94%, 95%, 96%, 97%, 98%, 99%, or 100% identical thereto; and a light chain CD R3 region comprising any one of SEQ ID NO: 68-72 or an amino acid sequence at least 70%, 75%, 80%, 85%, 90%, 91%, 92%, 93%, 94%, 95%, 96%, 97%, 98%, 99%, or 100% identical thereto. Preferably, the light chain comprises all of a CDR1 region, a CDR2 region, and a CDR3 region selected from SEQ ID NOs: 28-42 or SEQ ID NOs: 58-72 or an amino acid sequence at least 70%, 75%, 80%, 85%, 90%, 91%, 92%, 93%, 94%, 95%, 96%, 97%, 98%, 99%, or 100% identical thereto.

The heavy chain variable region of the antigen binding domain may comprise, consist of, or consist essentially of, SEQ ID NO: 153 or 155 or an amino acid sequence at least 70%, 75%, 80%, 85%, 90%, 91%, 92%, 93%, 94%, 95%, 96%, 97%, 98%, 99%, or 100% identical thereto. The light chain variable region of the antigen binding domain may comprise, consist of, or consist essentially of, SEQ ID NO: 154 or 156 or an amino acid sequence at least 70%, 75%, 80%, 85%, 90%, 91%, 92%, 93%, 94%, 95%, 96%, 97%, 98%, 99%, or 100% identical thereto. Accordingly, in an embodiment of the invention, the antigen binding domain comprises a heavy chain variable region comprising SEQ ID NO: 153 or an amino acid sequence at least 70%, 75%, 80%, 85%, 90%, 91%, 92%, 93%, 94%, 95%, 96%, 97%, 98%, 99%, or 100% identical thereto and/or a light chain variable region comprising SEQ ID NO: 154 or an amino acid sequence at least 70%, 75%, 80%, 85%, 90%, 91%, 92%, 93%, 94%, 95%, 96%, 97%, 98%, 99%, or 100% identical thereto. In an alternative embodiment, the antigen binding domain comprises a heavy chain variable region comprising SEQ ID NO: 155 or an amino acid sequence at least 70%, 75%, 80%, 85%, 90%, 91%, 92%, 93%, 94%, 95%, 96%, 97%, 98%, 99%, or 100% identical thereto and/or a light chain variable region comprising SEQ ID NO: 156 or an amino acid sequence at least 70%, 75%, 80%, 85%, 90%, 91%, 92%, 93%, 94%, 95%, 96%, 97%, 98%, 99%, or 100% identical thereto. Preferably, the antigen binding domain comprises both SEQ ID NOs: 153 and 154 or SEQ ID NOs: 155 and 156 or amino acid sequences at least 70%, 75%, 80%, 85%, 90%, 91%, 92%, 93%, 94%, 95%, 96%, 97%, 98%, 99%, or 100% identical thereto.

In an embodiment of the invention, the light chain variable region and the heavy chain variable region may be joined by a spacer or linker sequence. The linker may comprise any suitable amino acid sequence. In an embodiment of the invention, the linker may comprise, consist, or consist essentially of the amino acid sequence set forth in SEQ ID NO: 158 or an amino acid sequence at least 70%, 75%, 80%, 85%, 90%, 91%, 92%, 93%, 94%, 95%, 96%, 97%, 98%, 99%, or 100% identical thereto.

Additionally, the CAR may comprise a further spacer or linker sequence to connect the antigen binding domain with the transmembrane domain and spatially separate the antigen binding domain from the endodomain thereof. A flexible spacer or hinge region allows the antigen binding domain to orient in different directions to enable EphA3 binding. By way of example, hinge domains of antibodies, such as an IgG, IgA, IgM, IgE, or IgD antibodies, are also compatible for use in the chimeric receptors described herein. In some embodiments, the hinge domain is the hinge domain that joins the constant domains CH1 and CH2 of an antibody. Accordingly, the further spacer sequence may, for example, comprise an IgG1 Fc region, an IgG1 hinge or a CD8 stalk or hinge, or a combination thereof.

It is envisaged that the antigen binding domain can further include a leader or signal peptide sequence. The leader sequence may be a peptide sequence (e.g., about 5, about 10, about 15, about 20, about 25 or about 30 amino acids in length) present at the N-terminus of the newly synthesized protein (e.g., positioned adjacent the heavy chain variable region), which directs the protein into the secretory pathway. The leader sequence may comprise any suitable leader sequence known in the art, such as those derived from CD8, granulocyte-macrophage colony-stimulating factor (GM-CSF) receptor, CD28, murine kappa chain and CD16 In an embodiment, the leader sequence is a CD8 leader sequence. In this regard, the antigen binding domain may comprise a leader sequence comprising, consisting of, or consisting essentially of SEQ ID NO: 157 or an amino acid sequence at least 70%75%, 80%, 85%, 90%, 91%, 92%, 93%, 94%, 95%, 96%, 97%, 98%, 99%, or 100% identical thereto. In an embodiment of the invention, while the leader sequence may facilitate expression of the CAR on the surface of the cell, the presence of the leader sequence in an expressed CAR is not necessary in order for the CAR to function. Accordingly, upon expression of the CAR on the cell surface, the leader sequence may be cleaved off from the CAR. As such, in an embodiment of the invention, the CAR lacks a leader sequence.

The antigen binding domain of a CAR is commonly fused via a spacer and/or hinge region and transmembrane domain to an endodomain, which comprises or associates with an intracellular or cytoplasmic T-cell signalling domain. When the CAR binds the target-antigen, this results in the transmission of an activating signal to the T-cell it is expressed on. The endodomain is the portion of the CAR involved in signal-transmission and in this manner may comprise one or more co-stimulatory domains and/or one or more intracellular T-cell signalling domains.

Included in the scope of the invention are functional portions of the CARs described herein. The term “functionalportion” when used in reference to a CAR refers to any part or fragment of the CAR of the invention, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the invention are functional variants of the CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98%, about 99% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

The CARs of embodiments of the invention (including functional portions and functional variants) can be of any length, i.e., can comprise any number of amino acids, provided that the CARs (or functional portions or functional variants thereof) retain their biological activity (e.g., the ability to specifically bind to antigen, detect diseased cells in a mammal, or treat or prevent disease in a mammal, etc). For example, the CAR can be about 50 to about 5000 amino acids long, such as 50, 70, 75, 100, 125, 150, 175, 200, 300, 400, 500, 600, 700, 800, 900, 1000 or more amino acids in length.

Also provided is a cell comprising a CAR according to the invention. The CAR according to the present invention may be used to generate CAR-expressing immune cells, e.g., CAR T cells or CAR NK cells. Engineering of CARs into immune cells may be performed during culture, in vitro.

The antigen-binding region of the CAR of the present invention may be provided with any suitable format, e.g., scFv, scFab, etc.

### Nucleic Acids and Vectors

The present invention provides a nucleic acid, or a plurality of nucleic acids, encoding an antigen-binding molecule, polypeptide, or CAR according to the present invention.

In some embodiments, the nucleic acid is purified or isolated, e.g., from other nucleic acid, or naturally-occurring biological material. In some embodiments the nucleic acid(s) comprise or consist of DNA and/or RNA.

Thus, in another aspect, the present invention contemplates isolated nucleic acids that encode, or are complementary to a nucleic acid sequence which encodes, the isolated proteins (e.g., antibody and CAR proteins, inclusive of fragments, variants and derivatives thereof) disclosed herein.

Nucleotide sequences encoding the isolated proteins of the invention may be readily deduced from one or more of the complete nucleic acid sequences provided herein (see, e.g., SEQ ID NOs:1-12), although without limitation thereto.

This aspect also includes fragments, variants and derivatives of said isolated nucleic acid, such as those herein before described.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Accordingly, in particular embodiments, the isolated nucleic acid is cDNA.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

In one embodiment, nucleic acid variants encode a variant of an isolated protein of the invention.

In another embodiment, nucleic acid variants share at least 40%, 45%, 50%, 55%, 60% or 65%, 66%, 67%, 68%, 69%, preferably at least 70%, 71%, 72%, 73%, 74% or 75%, more preferably at least 80%, 81%, 82%, 83%, 84%, or 85%, and even more preferably at least 90%, 91%, 92%, 93%, 94%, 95%, 96%, 97%, 98% or 99% nucleotide sequence identity with an isolated nucleic acid of the invention.

In one particular embodiment, the isolated nucleic acid of the present aspect consists of: (a) a nucleic acid that: (i) encodes a segment, domain, portion or region of an antibody and/or an isolated CAR protein described herein, such as those according to SEQ ID NOS:13 to 156 and Table 1, and inclusive of variants or derivatives thereof; and (b) optionally one or more additional nucleic acid sequences. In this regard, the additional nucleic acid sequences can be heterologous nucleic acid sequences that can be at the 5′ (5-prime) and/or 3′ (3-prime) ends of the isolated nucleic acid sequence, although without limitation thereto.

The present invention also contemplates nucleic acids that have been modified such as by taking advantage of codon sequence redundancy. In a more particular example, codon usage may be modified to optimize expression of a nucleic acid in a particular organism or cell type.

The invention further provides use of modified purines (for example, inosine, methylinosine and methyladenosine) and modified pyrimidines (for example, thiouridine and methylcytosine) in nucleic acids of the invention.

It will be well appreciated by a person of skill in the art that the isolated nucleic acids of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In yet another embodiment, complementary nucleic acids hybridise to nucleic acids of the invention under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Complementary nucleotide sequences may be identified by blotting techniques that include a step whereby nucleotides are immobilized on a matrix (preferably a synthetic membrane such as nitrocellulose), a hybridization step, and a detection step, typically using a labelled probe or other complementary nucleic acid. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleotide sequence. An alternative blotting step is used when identifying complementary nucleic acids in a cDNA or genomic DNA library, such as through the process of plaque or colony hybridization. Other typical examples of this procedure are described in Chapters 8-12 of Sambrook et al., MOLECULAR CLONING. A Laboratory Manual (Cold Spring Harbor Press, 1989).

Methods for detecting labelled nucleic acids hybridized to an immobilized nucleic acid are well known to practitioners in the art. Such methods include autoradiography, chemiluminescent, fluorescent and colorimetric detection.

Nucleic acids may also be isolated, detected and/or subjected to recombinant DNA technology using nucleic acid sequence amplification techniques.

Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-β replicase amplification, recombinase polymerase amplification (RPA) and helicase-dependent amplification, although without limitation thereto.

As used herein, an “amplification product” refers to a nucleic acid product generated by nucleic acid amplification.

Nucleic acid amplification techniques may include particular quantitative and semi-quantitative techniques such as qPCR, real-time PCR and competitive PCR, as are well known in the art.

In some embodiments, the nucleic acid may be in a genetic construct that facilitates delivery and expression of the nucleic acid. In some embodiments, the present invention provides a vector, or plurality of vectors, comprising the nucleic acid or plurality of nucleic acids according to the present invention.

Accordingly, in yet another aspect, the invention provides a genetic construct comprising: (i) the isolated nucleic acid described herein; or (ii) an isolated nucleic acid comprising a nucleotide sequence complementary thereto. In one embodiment, the isolated nucleic acid is operably linked or connected to one or more regulatory sequences in a vector (e.g., an expression vector).

Suitably, the genetic construct is in the form of, or comprises genetic components of, a plasmid, bacteriophage, a cosmid, a yeast or bacterial artificial chromosome as are well understood in the art. Genetic constructs may be suitable for maintenance and propagation of the isolated nucleic acid in bacteria or other host cells, for manipulation by recombinant DNA technology and/or expression of the nucleic acid or an encoded protein of the invention.

For the purposes of host cell expression, the genetic construct can be an expression construct. Suitably, the expression construct comprises the nucleic acid of the invention operably linked to one or more additional sequences in an expression vector. A “vector” as used herein is a nucleic acid molecule used as a vehicle to transfer exogenous nucleic acid into a cell. The vector may be a vector for expression of the nucleic acid in the cell. An “expression vector” may be either a self-replicating extra-chromosomal vector such as a plasmid, or a vector that integrates into a host genome. In this regard, the vector may be capable of transferring a nucleic acid of the invention to a host cell, such as a T-cell, such that the cell expresses an EphA3-specific CAR or an EphA3 binding agent. To this end, the vector should ideally be capable of sustained high-level expression in T cells.

Such vectors may include a promotor sequence operably linked to the nucleotide sequence encoding the sequence to be expressed. A vector may also include a termination codon and expression enhancers.

By “operably linked” is meant that said additional nucleotide sequence(s) (e.g., regulatory nucleic acid sequences) is/are positioned relative to the nucleic acid of the invention preferably to initiate, regulate or otherwise control transcription. Typically, the selected nucleic acid sequence and regulatory nucleic acid sequence (e.g., promoter and/or enhancer) are covalently linked in such a way as to place the expression of nucleic acid sequence under the influence or control of the regulatory sequence (thereby forming an expression cassette).

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art are contemplated by the invention.

Suitable vectors include plasmids, binary vectors, DNA vectors, mRNA vectors, viral vectors, transposon-based vectors, and artificial chromosomes.

In particular embodiments, the expression vector is or comprises one or more viral delivery systems, such as adenovirus vectors, an adeno-associated virus (AAV) vectors, a herpesvirus vectors, a retrovirus vectors (e.g., gammaretroviral vectors; e.g., murine Leukemia virus (MLV)-derived vectors), a lentiviral vectors, vaccinia virus vectors, and a baculoviral vectors.

In some embodiments, the vector may be a eukaryotic vector, e.g., a vector comprising the elements necessary for expression of protein from the vector in a eukaryotic cell. In some embodiments, the vector may be a mammalian vector, e.g., comprising a cytomegalovirus (CMV) or SV40 promotor to drive protein expression.

In a further aspect, the invention provides a host cell transformed with a nucleic acid molecule or a genetic construct described herein.

Suitable host cells for expression may be prokaryotic or eukaryotic. For example, suitable host cells may include but are not limited to mammalian cells (e.g.m HeLa, HEK293T, Jurkat cells), yeast cells (e.g., Saccharomyces cerevisiae), insect cells (e.g., Sf9, Trichoplusia ni) utilized with or without a baculovirus expression system, plant cells (e.g., Chlamydomonas reinhardtii, Phaeodactylum tricornutum) or bacterial cells, such as E. coli. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2009), in particular Chapters 9 and 16.

### CAR-Expressing Cells

The present disclosure also provides a cell comprising or expressing a CAR according to the present disclosure. Also provided is a cell comprising or expressing a nucleic acid encoding a CAR according to the disclosure. Engineering of CARs into T-cells may be performed during culture, in vitro, for transduction and expression, such as happens during expansion of T-cells for adoptive T-cell therapy. Methods for engineering immune cells to express CARs are known to the skilled person and are described, for example, in Wang and Riviere, Mol Ther Oncolytics, (2016) 3: 16015, which is hereby incorporated by reference in its entirety. It will be appreciated that “at least one cell” encompasses a plurality of cells, e.g., a population of such cells.

The cell comprising or expressing a CAR according to the present disclosure may be a eukaryotic cell, e.g., a mammalian cell. The mammal may be a human, or a non-human mammal (e.g., rabbit, guinea pig, rat, mouse, or other rodent (including any animal in the order Rodentia), cat dog, pig, sheep, goat, cattle (including cows, e.g., dairy cows, or any animal in the order Bos), horse (including any animal in the order Equidae), donkey, and non-human primate).

In some embodiments, the cell may be from, or may have been obtained from, a human subject. Where the CAR-expressing cell is to be used in the treatment of a subject, the cell may be from the subject to be treated with the CAR-expressing cell (i.e., the cell may be autologous), or the cell may be from a different subject (i.e., the cell may be allogeneic).

In particular embodiments, the cell is or comprises an immune cell. The cell may be a cell of hematopoietic origin, e.g., a neutrophil, eosinophil, basophil, dendritic cell, lymphocyte, or monocyte. The lymphocyte may be, e.g., a T-cell, B cell, NK cell, NKT cell, or innate lymphoid cell (ILC), or a precursor thereof. The cell may express, e.g., CD3 polypeptides (e.g., CD3γ, CD3ε, CD3ζ, or CD3δ), TCR polypeptides (TCRα or TCRβ), CD27, CD28, CD4, or CD8.

Suitably, the immune cell is or comprises a T-cell inclusive of CD4+ helper T-cells and/or a CD8+ cytotoxic T-cells (e.g., a cytotoxic T-lymphocyte (CTL)). In this regard, the T-cell of the present aspect may be in a mixed population of CD4+ helper T-cell/CD8+ cytotoxic T-cells.

The use of CAR T-cells is associated with advantages that they can be systemically administered, and will home to both primary and metastasized tumours (see, Manzo et al., Human Mol Genetics (2015) R67-73).

In some embodiments, the cell is an antigen-specific T-cell. In embodiments of this type, an “antigen-specific” T-cell is a cell which displays certain functional properties of a T-cell in response to the antigen for which the T-cell is specific, or a cell expressing said antigen. In some embodiments, the properties are functional properties associated with effector T-cells, e.g., cytotoxic T-cells.

In some embodiments, an antigen-specific T-cell may display one or more of the following properties: cytotoxicity, e.g., to a cell comprising/expressing antigen for which the T-cell is specific; proliferation, IFN-γ expression, CD107a expression, IL-2 expression, TNF expression, perforin expression, granzyme expression, granulysin expression, and/or FAS ligand (FASL) expression, e.g., in response to antigen for which the T-cell is specific or a cell comprising/expressing antigen for which the T-cell is specific. Antigen-specific T-cells comprise a TCR capable of recognising a peptide of the antigen for which the T-cell is specific when presented by the appropriate MHC molecule. Antigen-specific T-cells may be CD4+ T-cells and/or CD8+ T cells.

In some embodiments, the antigen for which the T-cell is specific may be a peptide or polypeptide of a virus, e.g., Cytomegalovirus (CMV), Epstein-Barr virus (EBV), Adenovirus, human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

Advantageously, the isolated CAR of the present invention can be utilised in CAR gene transfer, an approach that is rapid, reliable and capable of generating large quantities of T-cells (>10⁸-10¹⁰ cells/patient) with specificity to EphA3, regardless of the patient's pre-existing immune repertoire. For example, retroviral or lentiviral transductions may require only 48 hours of culture with pre-activated T-cells. Further, large numbers of autologous T-cells can be obtained from leukaphoresis or isolation of peripheral blood mononuclear cells (PBMC) from a blood sample from a subject. Thus, it may be possible to engineer 10⁸-109 transformed or transfected T-cells for infusion in a few days.

Accordingly, a host cell (e.g., a T-cell) of the present invention can be used in the treatment of an EphA3-associated disease, disorder or condition, such as cancer, by means of adoptive transfer. To this end, T-cells are typically isolated from a biological sample taken from a subject, inclusive of donor subjects, for use in the adoptive transfer of genetically modified cells.

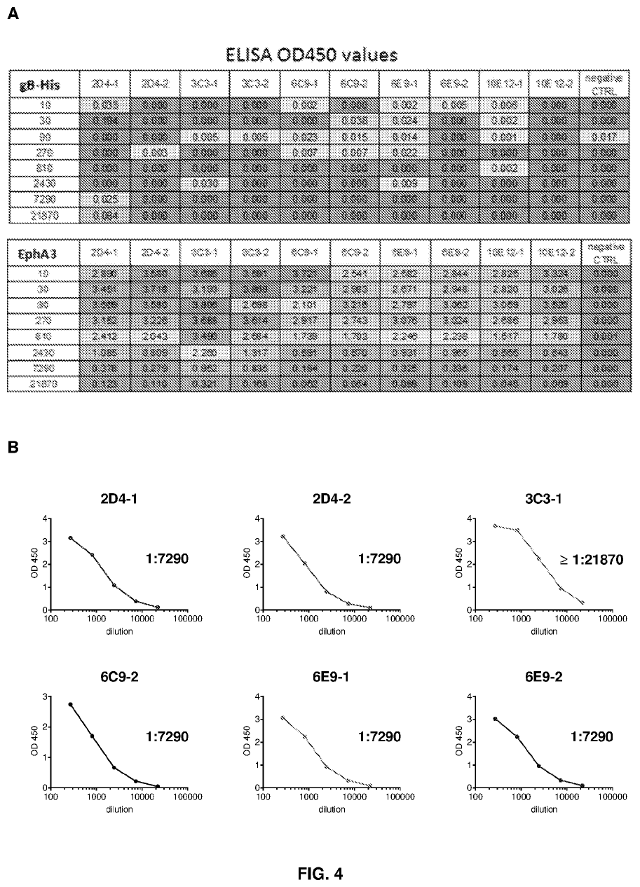

Preferably, the T-cells transduced or transformed with the CAR of the present invention (such as those CARs set forth in FIG. 4) contain a mixture of naive, central memory and effector memory cells.

In alternative embodiments, the host cell is, or is derived from, a stem cell, such as a haemopoietic stem cell (HSC). To this end, the host cell may therefore be a gene-modified stem cell, which, upon differentiation, produces a T-cell expressing a CAR of the invention.

In some embodiments, the host cell, such as a T cell, is genetically engineered to express a cytokine, chemokine and/or a receptor thereof.

To this end, CAR T-cells may be designed in several ways that enhance tumour cytotoxicity and specificity, evade tumour immunosuppression, avoid host rejection, and prolong their therapeutic half-life. TRUCK (T-cells Redirected for Universal Cytokine Killing) T-cells for example, possess a CAR but are also engineered to express and release cytokines such as IL-12 that promote tumour killing. Because these cells are designed to release a molecular payload upon activation of the CAR once localized to the tumour environment, these CART-cells are sometimes also referred to as “armoured CARs”. Exemplary cytokines include IL-2, IL-3. IL-4, IL-5, IL-6, IL-7, IL-10, IL-12, IL-13, IL-15, IL-18, M-CSF, GM-CSF, IFN-α, IFN-γ, TNF, TRAIL, FLT3 ligand, Lymphotactin, and TGF-β.

“Self-driving” or “homing” CART-cells are engineered to express a chemokine receptor in addition to their CAR. As certain chemokines can be upregulated in tumours, incorporation of a chemokine receptor aids in tumour trafficking to and infiltration by the adoptive T-cell, thereby enhancing both specificity and functionality of the CAR T-cell. Universal CAR T-cells also possess a CAR, but are engineered such that they do not express endogenous TCR (T-cell receptor) or MHC (major histocompatibility complex) proteins. Removal of these two proteins from the signalling repertoire of the adoptive T-cell therapy prevents graft-versus-host-disease and rejection, respectively. Armoured CART-cells are additionally so named for their ability to evade tumour immunosuppression and tumour-induced CAR T-cell hypofunction. These particular CAR T-cells possess a CAR, and may be engineered to not express checkpoint inhibitors. Alternatively, these CAR T-cells can be co-administered with a monoclonal antibody (mAb) that blocks checkpoint signalling. Administration of an anti-PDL1 antibody significantly restored the killing ability of CAR TILs (tumour infiltrating lymphocytes). While PD1-PDL1 and CTLA-4-CD80/CD86 signalling pathways have been investigated, it is possible to target other immune checkpoint signalling molecules in the design of an armoured CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armoured CAR T-cells may also be engineered to express proteins or receptors that protect them against or make them resistant to the effects of tumour-secreted cytokines. For example, CTLs (cytotoxic T lymphocytes) transduced with the double negative form of the TGF-β receptor are resistant to the immunosuppression by lymphoma secreted TGF-β. These transduced cells showed notably increased anti-tumour activity in vivo when compared to their control counterparts.

In yet another aspect, the invention provides a method of producing an isolated protein described herein (e.g., an isolated EphA3 binding agent or a CAR), comprising; (i) culturing the previously transformed host cell hereinbefore described; and (ii) isolating said protein from said host cell cultured in step (i).

The recombinant protein may be conveniently prepared by a person skilled in the art using standard protocols as for example described in Sambrook, et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. 1995-2009), in particular Chapters 10 and 16; and CURRENT PROTOCOLS IN PROTEIN SCIENCE Eds. Coligan et al., (John Wiley & Sons, Inc. 1995-2009), in particular Chapters 1, 5 and 6.

In a related aspect, the invention provides an isolated EphA3 binding agent or a CAR produced by the method of the aforementioned aspect.

In still a further aspect, the invention resides in an antibody or antibody fragment which binds and/or is raised against:
(i) the EphA3 binding agent of the first mentioned aspect; and/or
(ii) the CAR of the second mentioned aspect,
inclusive of fragments, variants and derivatives thereof.

Suitably, said antibody or antibody fragment specifically binds said isolated EphA3 binding agent or CAR. Preferably, the antibody or antibody fragment specifically or selectively binds or recognizes a full or partial amino acid sequence of a CDR, a V_{H} domain and/or a V_{L} domain described herein (e.g., SEQ ID NOs: 13-156). In this regard, the antibody or antibody fragment of the present aspect may be suitable for use in methods of detecting or isolating a T-cell that expresses the CAR having that particular CDR, V_{H} domain or V_{L} domain in a sample. To this end, antibodies and antibody fragments of the invention may be particularly suitable for affinity chromatography purification of the isolated EphA3 binding agents and CARs described herein. For example, reference may be made to affinity chromatographic procedures described in Chapter 9.5 of Coligan et al., supra.

Antibodies may be polyclonal or monoclonal, native or recombinant. Well-known protocols applicable to antibody production, purification and use may be found, for example, in Chapter 2 of Coligan et al., supra; and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative of the invention. For example, the antibodies may be polyclonal antibodies. Such antibodies may be prepared for example by injecting an isolated protein, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., supra, and in Harlow & Lane, 1988, supra.

Monoclonal antibodies may be produced using the standard method as for example, described in an article by Köhler & Milstein, 1975, Nature 256, 495, which is herein incorporated by reference, or by more recent modifications thereof as for example, described in Coligan et aL., supra by immortalizing spleen or other antibody producing cells derived from a production species which has been inoculated with one or more of the isolated proteins, fragments, variants or derivatives of the invention.

### CMV-Specific T-Cells

In certain aspects, provided herein are CMV-specific T-cells (e.g., CD4 T-cells and/or CD8 T-cells) that express a TCR (e.g., an αβ TCR or a γδ TCR) that recognises a peptide comprising a CMV epitope (e.g., a CMV epitope listed in Table 1). Thus, in some preferred embodiments, the T-cells of the invention are T-cells that recognise a peptide comprising a CMV epitope listed in Table 1.

TABLE 1
Exemplary CMV epitopes

| Epitope | HLA | hCMV | SEQ |
|---|---|---|---|
| Sequence | Restriction | Antigen | ID NO: |
| VTEHDTLLY | A*01:01 | pp50 | 181 |
| YSEHPTFTSQY | A*01:01 | pp65 | 182 |
| NLVPMVATV | A*02:01 | pp65 | 183 |
| VLEETSVML | A*02:01 | IE-1 | 184 |
| YILEETSVML | A*02:01 | IE-1 | 185 |
| AYAQKIFKIL | A*24:01; | IE-1 | 186 |
| | A*24:02 | | |
| QYDPVAALF | A*24:02 | pp65 | 187 |
| TPRVTGGGAM | B*07:02 | pp65 | 188 |
| RPHERNGFTVL | B*07:02 | pp65 | 189 |
| ELRRKMMYM | B*08:01 | IE-1 | 190 |
| ELKRKMIYM | B*08:01 | IE-1 | 191 |
| QIKVRVDMV | B*08:01 | IE-1 | 192 |
| DELRRKMMY | B*18:01; | IE-1 | 193 |
| | B*44:02 | | |
| IPSINVHHY | B*35:01 | pp65 | 194 |
| CPSQEPMSIYVY | B*35:08 | pp65 | 195 |
| CEDVPSGKL | B*40:01 | pp65 | 196 |
| HERNGFTVL | B*40:01 | pp65 | 197 |
| | B*40:02 | | |
| EEAIVAYTL | B*40:01 | IE-1 | 198 |
| | B*40:02 | | |
| QEFFWDANDIY | B*44:02 | pp65 | 199 |
| TRATKMQVI | C*06:02 | pp65 | 200 |
| YAYIYTTYL | B*41:01 | gB | 201 |
| QAIRETVEL | B*35:01 | pp65 | 202 |
| CRVLCCYVL | C*07:02 | pp65 | 203 |
| HELLVLVKKAQL | DRB1*11:01 | gH | 204 |
| AYSNTHSTRYV | DRB1*07 | gB | 205 |
| QEFFWDANDIYRIFA | DRB3*01:01 | pp65 | 206 |
| CMLTITTARSKYPYH | DRB1*04:01 | gH | 207 |
| PLKMLNIPSINVHHY | DRB1*01:01 | pp65 | 208 |
| EHPTFTSKYRIQGKL | DRB1*11:01 | pp65 | 209 |
| AGILARNLVPMVATV | DRB1*03:01 | pp65 | 210 |
| KARAKKDELR* | HLA-B*31:01 | IE-1 | 211 |

In some preferred embodiments of this type, the T-cell further comprises an antigen-binding molecule that binds to EphA3, as described above and/or elsewhere herein. In some embodiments, the T-cells provided herein can be engineered to express a CAR as described above and elsewhere herein. By way of an example, the CMV-specific T-cell further comprises an EphA3-binding CAR.

In some aspects, provided herein are methods of generating, activating, and/or inducing proliferation of T-cells (e.g., CTLs) that recognize one or more of the CMV epitopes described herein. In some embodiments, a sample comprising CTLs (e.g., a PBMC sample) are isolated, exposed to a pool of immunogenic peptides disclosed herein, and the stimulated CTLs harvested. Preferably, the pool of immunogenic peptides consists essentially of each of the CMV peptide epitope amino acid sequences set forth in Table 1. In certain embodiments, the exposed sample is incubated for at least 14 days. In some such embodiments, the exposed sample is incubated with IL-21 on Day 0. Preferably, the exposed sample is incubated with IL-2 on day 2. In more preferred embodiments, incubation of the exposed sample includes addition of IL-2 every three days.

In some embodiments, the PBMC sample is derived from a healthy donor. In certain embodiments, the PBMCs are derived from an immunocompromised donor. In some such embodiments, the donor is undergoing immunosuppressive therapy. In some embodiments, the donor is a solid organ transplant recipient. In further embodiments, the donor is receiving anti-viral therapy.

In some embodiments, a sample comprising CTLs (e.g., a PBMC sample) is incubated in culture with an APC that presents a peptide comprising a CMV epitope described herein on a class I MHC complex. The preparation of suitable APCs of this type is described, for example in the International PCT Patent Publication No. WO2019/220209, which is hereby incorporated by reference in its entirety. The APCs may be autologous to the subject from whom the T cells were obtained. In some embodiments, the sample containing T-cells is incubated two or more times with APCs provided herein. In some embodiments, the T-cells are incubated with the APCs in the presence of at least one cytokine, e.g., IL-2, IL-4, IL-7, IL-15, and/or IL-21. Exemplary methods for inducing proliferation of T-cells using APCs are provided, for example, in U.S. Pat. Pub. No. 2015/0017723, which is hereby incorporated by reference.

Expression of biomarkers by the CMV peptide-specific T-cells may be assessed by any suitable method, such as flow cytometry. In some embodiments, the CMV peptide-specific T-cells are stimulated by CMV-specific peptides and sorted via flow cytometry. Preferably, the CMV peptide-specific T-cells undergo stimulation and/or surface staining according to the protocols described in International PCT Patent Publication No. WO2019/220209, which is hereby incorporated by reference. In some embodiments, the CMV peptide-specific T-cells are incubated with one or more antibodies specific for CD107a, and subsequently sorted by flow cytometry. In some embodiments, the CMV peptide-specific T-cells are incubated with one or more antibodies that bind to intracellular cytokines, such as antibodies specific for IFN-γ, IL-2, and/or TNF. In some embodiments, the CMV peptide-specific T-cells are incubated with antibodies for intracellular cytokines and subsequently sorted via flow cytometry.

In some embodiments, the methods further comprise obtaining a sample comprising the T-cells from a donor subject (e.g., obtaining a PBMC sample from a donor subject). In some embodiments, the autologous T-cells (e.g., CD4+ T-cells or CD8+ T-cells) are isolated form the sample. In some embodiments, the sample is comprised mostly or completely of allogeneic T-cells.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T-cells (e.g., CTLs) in the sample express CD107a.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T-cells (e.g., CTLs) in the sample express IFN-γ.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T-cells (e.g., CTLs) in the sample express TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, 50%, 60%, 70%, 80% or 90% of the T-cells (e.g., CTLs) in the sample express IL-2.

In some embodiments, at least 1%, 2%, 3%, 4%, 5%, 6%, 7%, 8%. 9%, 10%, 11/%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CID107a and IFN-γ.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CID107a and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CD107a and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express IFN-γ and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express IFN-γ and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express TNF and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81/%, 82%, 83%, 84%, 85%, 86%, 87%, 88%, 89%, or 90% of the T-cells (e.g., CTLs) in the sample express IFN-γ, TNF, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CDi107a, TNF, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CD107a, IFN-γ, and IL-2.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CD107a, IFN-γ, and TNF.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) in the sample express CD107a, IFN-γ, TNF, and IL-2.

In some embodiments of the methods disclosed herein, the T-cells (e.g., CTLs) display reactivity against multiple peptide epitopes derived from multiple CMV antigens. In this regard, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80% 81%, 82%, 83%, 84%, 85%, 86%, 87%, 88%, 89%, or 90% of the T-cells (e.g., CTLs) are reactive to more than one CMV epitope. In certain embodiments, the T-cells (e.g., CTLs) are reactive to any one of the CMV peptide epitope amino acid sequences set forth in Table 1, or combinations thereof. In some embodiments, the T-cells (e.g., CTLs) are reactive to any one of pp50, pp65, IE-I, gB, gH, or combinations thereof.

T-cell biomarker expression and/or CMV reactivity may be measured and/or analysed either before or after T-cell (e.g., CTL) expansion by any one of the methods disclosed herein, e.g., by exposure to a pool of immunogenic CMV peptide epitopes.

In some embodiments, CMV reactivity and biomarker expression is quantified prior to stimulation of the T-cells (e.g., CTLs). Alternatively or additionally, CMV reactivity and biomarker expression may be quantified after stimulation of the T-cells (e.g., CTLs). In some embodiments, CMV reactivity is measured by quantifying the percentage of T-cells in the sample that express CD107a. In some embodiments, CMV reactivity is measured by quantifying the percentage of T-cells in the sample that express IFN-γ. In some embodiments, CMV reactivity is measured by quantifying the percentage of T-cells in the sample that express TNF. In some embodiments, CMV reactivity is measured by quantifying the percentage of T-cells in a sample that express IL-2. In some embodiments, CMV reactivity is measured as a percentage of T-cells that express multiple biomarkers (e.g., two or more of CD107a, IFN-γ, TNF, and IL-2, preferably all four). In some embodiments, the CMV reactivity is calculated by quantifying the percentage of T-cells in a sample that express CD107a, IFN-γ, TNF, and IL-2. T-cells may be isolated from a sample (e.g., a PBMC sample or a sample comprising T-cells) either before or after CMV reactivity percentage quantification. Therefore, in some embodiments, CMV reactivity is the percentage of T-cells having the desired characteristic(s) in a sample that comprises mostly T-cells.

In some embodiments, CMV reactivity is measured by quantifying the percentage of CD8+ lymphocytes in the sample that express CD107a. In some embodiments, CMV reactivity is measured by quantifying the percentage of CD8+ lymphocytes in the sample that express IFN-γ. In some embodiments, CMV reactivity is measured by quantifying the percentage of CD8+ lymphocytes in the sample that express TNF. In some embodiments, CMV reactivity is measured by quantifying the percentage of CD8+ lymphocytes in a sample that express IL-2. In some embodiments, CMV reactivity is measured as a percentage of CD8+ lymphocytes that express multiple biomarkers (e.g., two or more of CD 107a, IFN-γ, TNF, and IL-2, preferably all four). CD8+ lymphocytes may be isolated from a sample (e.g., a PBMC sample or a sample of CD8+ lymphocytes) either before or after CMV reactivity percentage quantification. Therefore, in some embodiments, CMV reactivity is the percentage of CD8+ lymphocytes having the desired characteristic(s) in a sample that comprises mostly or CD8+ lymphocytes.

In some embodiments, CMV reactivity is measured by quantifying the percentage of CD3+ lymphocytes in the sample that express CD107a. In some embodiments, CMV reactivity is measured by quantifying the percentage of CD3+ lymphocytes in the sample that express IFN-γ. In some embodiments, CMV reactivity is measured by quantifying the percentage of CD3+ lymphocytes in the sample that express TNF. In some embodiments, CMV reactivity is measured by quantifying the percentage of CD3+ lymphocytes in a sample that express IL-2. In some embodiments, CMV reactivity is measured as a percentage of CD3+ lymphocytes that express multiple biomarkers (e.g., two or more of CID107a, IFN-γ, TNF, and IL-2, preferably all four). CD3+ lymphocytes may be isolated from a sample (e.g., a PBMC sample or a sample of CD3+ lymphocytes) either before or after CMV reactivity percentage quantification. Therefore, in some embodiments, CMV reactivity is the percentage of CD3+ lymphocytes having the desired characteristic(s) in a sample that comprises mostly CD3+ lymphocytes.

In some of the most preferred embodiments of the invention, the T-cells present an EphA3 antigen-binding molecule on its surface. For example, the T-cell may present an EphA3-binding CAR on its surface.

The T-cells may be autologous or not autologous to the subject. In some embodiments, the T-cells are stored in a cell bank before they are administered to the subject. In some preferred embodiments, the T-cells are allogeneic to the subject.

### Pharmaceutical Compositions

In still yet another aspect, the invention provides a composition comprising the EphA3 binding agent described herein, the CAR described herein, the isolated nucleic acid described herein, the genetic construct described herein and/or the host cell described herein and a pharmaceutically acceptable carrier, diluent or excipient.

In some aspects provided herein is a composition (e.g., a pharmaceutical composition) comprising a CMV specific CTL that expresses or presents an EphA3 CAR, or preparation thereof, formulated together with a pharmaceutical carrier, as well as methods of administering such pharmaceutical compositions.

By “pharmaceutically-acceptable carrier, diluent or excipient” is meant a solid or liquid filler, diluent or encapsulating substance that may be safely used in systemic administration.

In some embodiments, the composition may further comprise an adjuvant. As used herein, the term “adjuvant” broadly refers to an immunological or pharmacological agent that modifies or enhances the immunological response to a composition in vitro or in vivo. For example, an adjuvant might increase the presence of an antigen over time, help absorb an antigen-presenting cell antigen, activate macrophages and lymphocytes and support the production of cytokines. By changing an immune response, an adjuvant might permit a smaller dose of the immune interacting agent or preparation to increase the dosage effectiveness or safety. For example, an adjuvant might prevent T-cell exhaustion and thus increase the effectiveness or safety of a particular immune interacting agent or preparation. Examples of adjuvants include, but are not limited to, an immune modulatory protein, Adjuvant 65, α-GalCer, aluminium phosphate, aluminium hydroxide, calcium phosphate, β-Glucan Peptide, CpG DNA, GPI-0100, lipid A and modified versions thereof (e.g., monophosphorylated lipid A), lipopolysaccharide, Lipovant, Montanide, N-acetyl-rmuramyl-L-alanyl-D-isoglutamine, Pam3CSK4, Quil-A, and trehalose dimycolate.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Depending upon the particular route of administration, a variety of carriers, well known in the art may be used. These carriers may be selected from a group including sugars, starches, cellulose and its derivatives, malt, gelatine, talc, calcium sulphate, vegetable oils (such as olive oil), synthetic oils, polyols (such as glycerol, propylene glycol, polyethylene glycol, and the like), alginic acid, phosphate buffered solutions, emulsifiers, isotonic saline and salts such as mineral acid salts including hydrochlorides, bromides and sulphates, organic acids such as acetates, propionates and malonates and pyrogen-free water. Further examples of suitable aqueous and non-aqueous carriers which may be employed in the pharmaceutical compositions of the invention include water, ethanol, and suitable mixtures thereof, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

A useful reference describing pharmaceutically acceptable carriers, diluents and excipients is Remington's Pharmaceutical Sciences (Mack Publishing Co. N.J. USA, 1991) which is incorporated herein by reference.

### Therapeutic Applications

Aspects of the present disclosure are concerned in particular with the use of the antigen-binding agents, and/or cells as described herein, in the treatment of a cancer in a subject.

Accordingly, the present disclosure provides a method of treating or preventing a cancer in a subject, said method including the step of administering to the subject a therapeutically effective amount of the EphA3 binding agents described herein, or at least one T-cell comprising a chimeric antigen receptor (CAR) specific for EphA3 as described herein, or the compositions described, herein, to thereby treat or prevent the cancer in the subject.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto. In particular embodiments, the cancer is a solid cancer, such as glioblastoma multiforme. Suitably, the cancer expresses, such as overexpresses, EphA3.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. Treatment or alleviation of a cancer may be effective to prevent progression of the cancer e.g., to prevent worsening of the condition or to slow the rate of development of a more severe disease state. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

In some embodiments, about 1×10⁵ to about 1×10⁸ T-cells are administered to the subject per dose of T-cells. In some embodiments, about 1×10⁶ to about 1×10⁷ T-cells are administered to the subject per dose of T-cells. In some embodiments, 1×10⁶, 1×10⁷, 1.5×10⁷, or 2×10⁷ T-cells (e.g., CTLs) are administered to the subject. Multiple doses may be administered to the subject. In some embodiments, an initial dose of T-cells (e.g., autologous CTLs) is administered, and one or more additional doses of T-cells (e.g., autologous CTLs) are administered, e.g., at increasing doses along the course of therapy. In some embodiments, two or more, three or more, four or more, five or more, six or more, seven or more, eight or more, nine or more, or ten or more doses are administered. The subject may be administered additional doses that are the same or different from the initial dose. For example, a lower dose may be administered followed by a higher dose. The doses may be administered daily, twice a week, weekly, biweekly, once a month, once every two months, once every three months, or once every six months. In some embodiments, the subject does not experience any adverse effects as a result of T-cell (e.g., allogeneic CTL) administration.

The term “therapeutically effective amount” describes a quantity of a specified agent, such as an EphA3 binding agent or CAR, sufficient to achieve a desired effect in a subject being treated with that agent. For example, this can be the amount of a composition comprising one or more EphA3 binding agents and/or CARs described herein, necessary to reduce, alleviate and/or prevent a cancer or cancer associated disease, disorder or condition, inclusive of cancer metastasis and recurrence. In some embodiments, a “therapeutically effective amount” is sufficient to reduce or eliminate a symptom of a cancer. In other embodiments, a “therapeutically effective amount” is an amount sufficient to achieve a desired biological effect, for example an amount that is effective to decrease or prevent cancer growth, recurrence and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

It will be appreciated that the method of the present aspect may include one or more further cancer treatments in addition to those recited above. Such cancer treatments may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g., antibodies, inhibitory nucleic acids such as siRNA) or chemotherapeutic agents are referred to herein as “anti-cancer therapeutic agents” or “anti-cancer agents”.

In some embodiments, the subject is also administered an anti-cancer compound. Exemplary anti-cancer compounds include, but are not limited to, Alemtuzumab (Campath®), Alitretinoin (Panretin®), Anastrozole (Arimidex®), Bevacizurmab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afmitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Peijeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 13 II-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-afliberoept (Zaltrap®).

In some embodiments, the subject is also administered a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma (1,1) and calicheamicin omega (1,1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the subject is also administered an immunotherapeutic agent. Immunotherapy refers to a treatment that uses a subject's immune system to treat or prevent a condition, e.g. cancer vaccines, cytokines, use of target-specific antibodies, T-cell therapy, and dendritic cell therapy.

In some embodiments, the subject is also administered an immune modulatory protein. Examples of immune modulatory proteins include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-γ”), Interleukin-1 alpha (IL-1α”), Interleukin-1 beta (“IL-1p”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1a”), Chemokine (C—C motif) ligand 4 (“MIP-1P”), Macrophage inflammatory protein-1-delta (“MIP-1”), Platelet-derived growth factor subunit B (“PDGF-BB”), Chemokine (C—C motif) ligand 5, Regulated on Activation, Normal T-cell Expressed and Secreted (“RANTES”), TEMP metallopeptidase inhibitor 1 (“TIMP-1”), TIMP metallopeptidase inhibitor 2 (“TIMP-2”), Tumour necrosis factor, (“TNF”), Tumour necrosis factor, lymphotoxin-beta (“TNF-β”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“GFBP-6”), Insulin-like growth factor 1 (“IGF-1”), Insulin, Macrophage colony-stimulating factor (“M-CSFR”), Nerve growth factor receptor (“NGFR”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCFR”), Transforming growth factor alpha (“TGFα”), Transforming growth factor beta-1 (“TGFβ1”), Transforming growth factor beta-3 (“TGFβ3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor FIFO (“Axl”), Betacellulin (“BTC”), Mucosae-associated epithelial chemokine (“CCL28”), Chemokine (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-1”), Chemokine (C—C motif) ligand 16 (“HCC-4”), Interleukin-9 (“IL-9”), Interleukin-17F (“IL-17F”), Interleukin-18-binding protein (“IL-18 BPa”), Interleukin-28A (“IL-28A”), Interleukin 29 (“IL-29”), Interleukin-31 (“IL-31”), C—X—C motif chemokine 10 (“IP-10”), Chemokine receptor CXCR3 (“I-TAC”), Leukaemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3a”), C—C motif chemokine 19 (“MIP-3p”), Chemokine (C—C motif) ligand 23 (“MPIF-1”), Macrophage stimulating protein alpha chain (“MSPa”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1a”), Chemokine (C—C motif) ligand 17 (“TRC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumour necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumour necrosis factor receptor superfamily member 1 (“GITR”), Tumour necrosis factor receptor superfamily member 14 (“HVEM”), Intercellular adhesion molecule 3 (“ICAM-3”), IL-1 R4, IL-1 Rβ, IL-10RP, IL-17R, IL-2Ry, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGI-pI, Beta-type platelet-derived growth factor receptor (“PDGF Rp”), Platelet endothelial cell adhesion molecule (“PECAM-l”), RAGE, Hepatitis A virus cellular receptor 1 (“TIM-1”), Tumour necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 RI, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-p”), sgp130, Secreted frizzled-related protein 2 (“ShhN”), Sialic acid-binding immunoglobulin-type lectins (“Siglec-5”), ST2, Transforming growth factor-beta 2 (“TGF p32”), Tie-2, Thrombopoietin (“TPO”), Tumour necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“BP2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C—X—C motif) ligand 1 (“GROα), human chorionic gonadotropin (“p HCG”), Insulin-like growth factor 1 receptor (“IGF-1 sR”), IL-1 sRII, IL-3, IL-18 RP, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, tumour necrosis factor ligand superfamily member 13, Acidic leucine-rich nuclear phosphoprotein 32 family member B (“APRIL”), Bone morphogenetic protein 2 (“BMP-2”), bone morphogenetic protein 9 (“BMP-9”), Complement component 5a (“C5a”), Cathepsin L, CD200, CD97, Chemerin, Tumour necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGFR”), IFN-α/p R2, Insulin-like growth factor 2 (“IGF-2”), Insulin-like growth factor 2 receptor (“IGF-2R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumour necrosis factor receptor superfamily member 10A (“TRAIL RI”), Transferrin (“TRF”), WIF-1ACE-2, Albumin, AMICA, Angiopoietin 4, B-cell activating factor (“BAFF”), Carbohydrate antigen 19-9 (“CA19-9”), CD163, Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-I”), GPCR-associated sorting protein 2 (“GASP-2”), Granulocyte colony-stimulating factor receptor (“GCSFR”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulphate proteoglycan (“Syndecan-1”), Tumour necrosis factor receptor superfamily member 13B (“TACI”), Tissue factor pathway inhibitor (“TFP”), TSP-I, Tumour necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signalling pathway protein 1 (“WISP-1”), and Receptor Activator of Nuclear Factor k B (“RANK”).

In some embodiments, the subject is also administered an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen-binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein (e.g., a vaccine composition provided herein) is administered prophylactically to prevent cancer and/or a CMV infection. In some embodiments, the vaccine is administered to inhibit tumour cell expansion. The vaccine may be administered prior to or after the detection of cancer cells or CMV infected cells in a patient. Inhibition of tumour cell expansion is understood to refer to preventing, stopping, slowing the growth, or killing of tumour cells. In some embodiments, after administration of a vaccine comprising peptides, nucleic acids, antibodies or APCs described herein, a proinflammatory response is induced. The proinflammatory immune response comprises production of proinflammatory cytokines and/or chemokines, for example, IFN-γ and/or IL-2. Proinflammatory cytokines and chemokines are well known in the art.

Combination therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

By “administering” or “administration” is meant the introduction of an isolated EphA3 binding agent, CAR, encoding nucleic acid, genetic construct, cell or composition disclosed herein into an animal subject by a particular, chosen route.

Administration of the EphA3 binding agent, CAR or variant thereof, or an encoding nucleic acid, or a genetic construct, or cell, or a composition comprising same, may be by any known parenteral, topical or enteral route inclusive of intravenous, intramuscular, intraperitoneal, intracranial, transdermal, oral, intranasal, anal and intra-ocular, although without limitation thereto.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

In another related aspect, the invention resides in use of the EphA3 binding agent described herein, the CAR described herein, the isolated nucleic acid described herein, the genetic construct described herein and/or the host cell described herein in the manufacture of a medicament for the prevention and/or treatment of a cancer in a subject.

In one embodiment, the cancer is or comprises glioblastoma multiforme.

### Labels and Conjugates

In still another aspect, the invention provides a method of detecting EphA3 or a cell expressing EphA3, said method including the step of forming a complex between the EphA3 binding molecule or the CAR hereinbefore described and EphA3 to thereby detect EphA3 or the cell expressing EphA3.

In one embodiment, the method includes the initial step of contacting the EphA3 or the cell expressing EphA3 with the EphA3 antigen-binding molecule or the CAR described above or elsewhere herein.

Thus, in some embodiments the antigen-binding molecule of the present invention additionally comprise a detectable moiety.

In certain embodiments, the cell is or comprises a cancer cell.

It will therefore be understood that an EphA3 binding agent or CAR disclosed herein may be used to assist medical diagnosis of cancer. Suitably, the method includes detecting EphA3, such as when expressed by cancer cells present in, or obtained from, a biological sample. In certain embodiments, the biological sample may be a pathology sample that comprises one or more fluids, cells, tissues, organs or organ samples obtained from a human. Non-limiting examples include blood, plasma, saliva, serum, lymphocytes, urine, faeces, amniotic fluid, cervical samples, cerebrospinal fluid, tissue biopsies, bone marrow and skin, although without limitation thereto.

In some embodiments, the antigen-binding molecule comprises a detectable moiety. For example, the EphA3 antigen-binding molecule and/or CAR is labelled with a fluorescent label, phosphorescent label, luminescent label, immunodetectable label (e.g., an epitope tag), radiolabel, chemical, nucleic acid or enzymatic label. The antigen-binding molecule may be covalently or non-covalently labelled with the detectable moiety.

Fluorescent labels include e.g., fluorescein, rhodamine, allophycocyanin, eosine and NDB, green fluorescent protein (GFP), chelates of rare earths (such as europium (Eu), terbium (Tb) and samarium (Sm)), tetramethyl rhodamine, Texas Red, 4-methyl umbelliferone, 7-amino-4-methyl coumarin, Cy3, and Cy5.

Radiolabels include radioisotopes such as iodine¹²³, iodine¹²⁵, iodine¹²⁶, iodine¹³¹, iodine¹³³, bromine⁷⁷, technetium^{99m}, indium¹¹¹, indium^{113m}, gallium⁶⁷, gallium⁶⁸, ruthenium⁹⁵, ruthenium¹⁰³, ruthenium¹⁰⁵, mercyry²⁰⁷, mercury²⁰³, rhenium^{99m}, rhenium¹⁰¹, rhenium¹⁰⁵, scandium⁴⁷, tellurium^{121m}, tellurium^{122m}, tellurium¹²⁵, thulium¹⁶⁵, thulium¹⁶⁷, thulium¹⁶, copper⁶⁷, fluorine¹⁸, Yttrium⁹⁰, Palladium¹⁰⁰, Bismuth²¹⁷ and Antimony²¹¹.

Luminescent labels include as radioluminescent, chemiluminescent (e.g., acridinium ester, luminol, isoluminol) and bioluminescent labels. Immunodetectable labels include haptens, peptides/polypeptides, antibodies, receptors and ligands such as biotin, avidin, streptavidin or digoxigenin. Nucleic acid labels include aptamers. Enzymatic labels include e.g., peroxidase, alkaline phosphatase, glucose oxidase, beta-galactosidase and luciferase.

In some embodiments the antigen-binding molecules of the present invention are conjugated to a chemical moiety. The chemical moiety may be a moiety for providing a therapeutic effect. Antibody-drug conjugates are reviewed, e.g., in Parslow et al., Biomedicines. 2016 September; 4(3): 14. In some embodiments, the chemical moiety may be a drug moiety (e.g., a cytotoxic agent). In some embodiments, the drug moiety may be a chemotherapeutic agent.

The label may be selected from a group including biotin, avidin, digoxigenin, an enzyme (e.g., alkaline phosphatase or horseradish peroxidase), a fluorophore (e.g., FITC, Texas Red, Coumarin), a radioisotope (e.g., ¹²⁵1, ¹³1, ⁶⁷Ga, ¹¹¹In) and/or a direct visual label (e.g., a gold particle), although without limitation thereto.

Suitably, detection of EphA3 includes the step of forming a detectable complex between an EphA3 binding agent or CAR and EphA3 or a cell expressing EphA3. The complex so formed may be detected by any technique, assay or means known in the art including immunoblotting, immunohistochemistry, immunocytochemistry, immunoprecipitation, ELISA, flow cytometry, magnetic bead separation, biosensor-based detection systems such as surface plasmon resonance and imaging such as PET imaging, although without limitation thereto.

To facilitate detection the EphA3 binding agent or CAR may be directly labelled as hereinbefore described or a labelled secondary antibody may be used. The labels may be as hereinbefore described.

In some embodiments, a detection kit may be provided which comprises an antibody or antibody fragment disclosed herein together with one or more detection reagents such as enzymes, enzyme substrates (e.g., Luminol, AMPPD, NBT), secondary antibodies and/or magnetic beads although without limitation thereto.

In another aspect, the invention provides an isolated protein comprising, consisting essentially of or consisting of an amino acid sequence set forth in any one of SEQ ID NOS: 13 to 156 and/or Tables 4-7 or an amino acid sequence at least 70% identical thereto.

In a final aspect, the invention provides an isolated nucleic acid comprising, consisting or consisting essentially of a nucleic acid sequence set forth in any one of SEQ ID NOS: 1 to 12 and/or Table 3 or a nucleic acid sequence at least 70% identical thereto.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). In some embodiments, the subject is a human.

So that preferred embodiments may be described in detail and put into practical effect, reference is made to the following non-limiting Examples.

### EXAMPLES

### Example 1

### Rationale

Adoptive immunotherapy with gene-modified T-cells expressing chimeric antigen receptors (CARs) have shown substantial success in treating blood cancers.⁴ Despite these breakthroughs the success of CAR T-cells in treating solid tumours has been limited.

CARs utilize tumour targeting specificity of any antibody, or receptor ligand, to redirect the cytolytic potency of T-cells. The therapeutic value lies in the tailored engineering of the binding region to target specific cancer biomarkers, or a combination of markers, for on-tumour and low off-target activity. In GBM, and a number of other cancers, EphA3 has been identified as a therapeutic target.⁶ EphA3 is overexpressed in a cancers and is associated with tumour growth, invasiveness, and metastasis.⁶⁻⁹ EphA3 appears critical in maintaining tumour cells in a less differentiated state and promotes self-renewal of cancer stem cells (CSC). Targeted inhibition of EphA3 is therefore a promising therapeutic approach to treat solid cancers, and, by targeting CSC, potentially effective for heterogeneous, metastatic or cancers considered resistant to therapy.

An EphA3-targeting therapeutic antibody is currently under clinical assessment in recurrent glioblastoma patients, is well tolerated, and demonstrated promising clinical activity in specific cancer cohorts (10). However, given the challenges in achieving and maintaining pharmacological levels of inhibitors, particularly in the brain (11), the present Example investigates an approach using CAR T-cells that could potentially surpass traditional strategies and deliver a targeted anti-tumour response in the brain.

### Design

### EphA3 Monoclonal Antibodies

The extracellular domain sequence of human EphA3 (P29320, 21-541 aa) was designed, optimised, synthesised and sub-cloned into the pcDNA3.4 vector. Transfection grade plasmid was maxi-prepared for Expi293 cell expression. The cloning strategy is shown in FIG. 1.

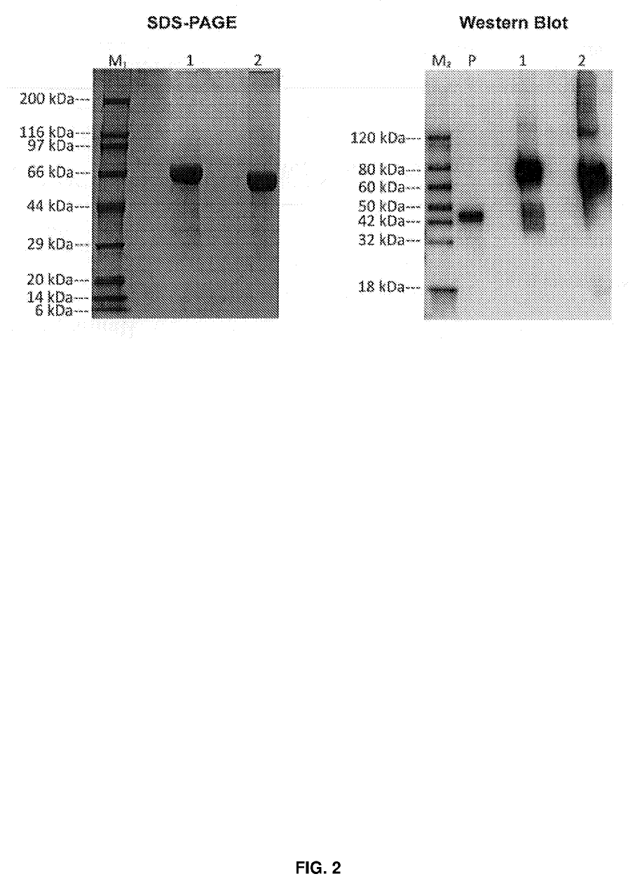

Expi293F cells were grown in serum-free Expi293™ Expression Medium in Erlenmeyer flasks at 37° C. with 8% C02 on an orbital shaker. On the day of transfection, DNA and transfection reagent were mixed at an optimal ratio and then added into the flask. The cell culture supernatant was collected on day 6 and loaded onto an affinity purification column for purification. After washing and elution with appropriate buffers, the eluted fractions were pooled and buffer exchanged to final formulation buffer. The purified protein was analysed by SDS-PAGE and Western blotting for molecular weight and purity measurements (FIG. 2). The concentration was determined by BCA™ assay with BSA as a standard, 1.77 mg/mL protein with approximately 95% purity was obtained and stored at −80° C. in multiple aliquots to avoid multiple freeze-thaws.

Three BALB/c and three C57 mice were immunized with the recombinant human EphA3 protein following the immunization schedule shown in table below.

TABLE 2
Immunization schedule of EphA3 (P29320|21-541)

| Procedure | Schedule | Dosage and route |
|---|---|---|
| Pre-immune bleed | Day −4 | |
| Primary immunization | Day 0 | 50 μg/animal, sc |
| Boost 1 | Day 14 | 25 μg/animal, sc |
| Test bleed 1 | Day 21 | |
| Boost 2 | Day 28 | 25 μg/animal, sc |
| Test bleed 2 | Day 35 | |
| Final boost | Day 50 | 25 μg/animal, sc |
| Cell fusion | Day 54 | |

sc—subcutaneously

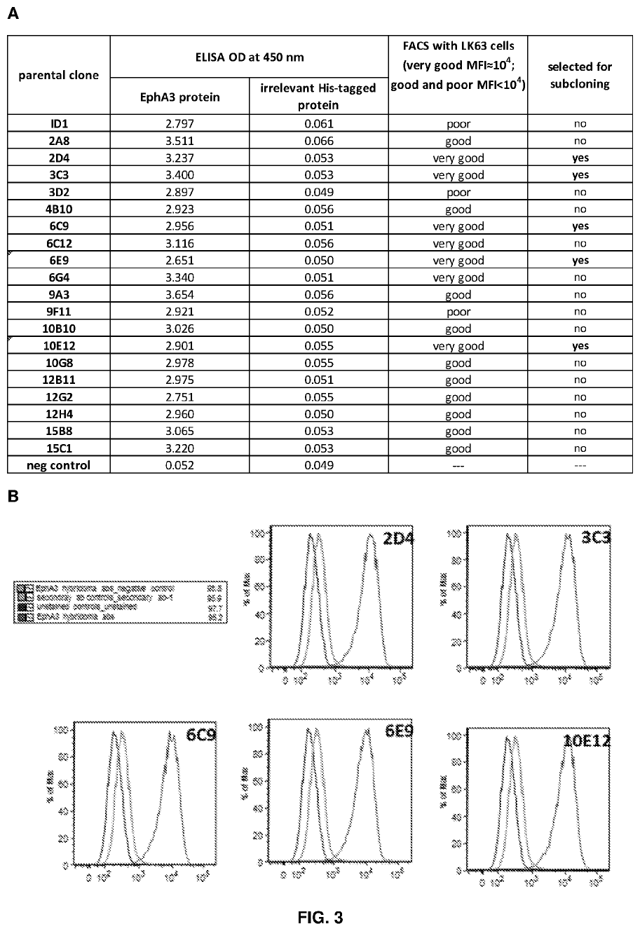

Cell fusion and clone plating was performed by electro-fusion for each group of animals. All fused cells from each fusion were plated into 96-well plates and conditioned media screened by ELISA with EphA3 protein. Positive supernatants were confirmed to be negative for irrelevant his-tagged protein by ELISA. Five parental hybridoma clones were selected for subcloning, based on EphA3 specificity. Ten monoclonal subclone supernatants were screened for EphA3 binding efficiency to recombinant EphA3 in ELISA, or EphA3-expressing leukemic cell line LK63⁹ by flow cytometry (FIGS. 3 and 4). Hybridomas 3C3-1 and 2D4-1 were selected to be sequenced, whilst clones with less binding efficiency (such as 6C9-1), were excluded.

For sequencing, the total RNA was isolated from 3C3-1 and 2D4-1 hybridoma cells using TRIzol® Reagent. Total RNA was then reverse-transcribed into cDNA using either isotype-specific anti-sense primers or universal primers using PrimeScript™ 1st Strand cDNA Synthesis Kit. Antibody fragments of heavy chain and light chain were amplified by rapid amplification of cDNA ends (RACE). Amplified antibody fragments were cloned into a standard cloning vector separately. Colony PCR was performed to screen for clones with inserts of correct sizes and the consensus sequence listed in Table 3.

TABLE 3
Complementarity Determining Region Nucleic Acid Sequences

| | | SEQ ID |
|---|---|---|
| | DNA sequence | NO: |
| 3C3-1 | | |
| V_{H} | CDR1: | 1 |
| | ATCTACACGATACAC | |
| | CDR2: | 2 |
| | TACATTAATCCTAGCAGTGATTATACTAATTACAATCAGAAGTTCAAGGAC | |
| | CDR3: | 3 |
| | GAAGGGGCGTATGGTAACTACGACTTTGCTATGGACTAC | |
| V_{L} | CDR1: | 4 |
| | AGATCTAGTCAGAACATTGTCCATAATAATGGAAACACCTATTTAGAA | |
| | CDR2: | 5 |
| | AAAGTTTCCAACCGATTTTCT | |
| | CDR3: | 6 |
| | TTTCAAACTTCACATGTTCCGTACACG | |
| 2D4-1 | | |
| V_{H} | CDR1: | 7 |
| | GGCTATGGTGTAAAC | |
| | CDR2: | 8 |
| | ATGATATGGGGTGATGGAAGCACAGACTATAATTCAGTTCTCAAATCC | |
| | CDR3: | 9 |
| | GATCGGGGTATTAGTTATTACTATGCTATGGACTAC | |
| V_{L} | CDR1: | 10 |
| | AGGGCCAGTAAAAGTGTCAGTGCATCTGGCTATAGTTATTTGCAC | |
| | CDR2: | 11 |
| | CTTGCATACAACCTAGGATCT | |
| | CDR3: | 12 |
| | CAGCACAGTAGGGAGTTTCCGCTCACG | |

The complementarity-determining regions (CDRs) of 303-1 and 2D4-1 are listed in Tables 4-7.

We have generated various EphA3-specific high-affinity complementarity-determining regions (CDRs). These unique sequences form EphA3-specific binding domains and can be used to generate a single-chain variable fragment (scFv) to target EphA3 using CAR T-cell technology, or other applications where EphA3 is the target.

TABLE 4
Clone 3C3-1 Heavy Chain CDR1, 2 and 3 amino acid sequences

| | | | | | SEQ |
|---|---|---|---|---|---|
| | | | | | ID |
| Region | Definition | Sequence Fragment | Residues | Length | NO: |
| HFR1 | Chothia | QVQLQQSGAELARPGASVKMSCKSS----- | 20-44 | 25 | 73 |
| | AbM | QVQLQQSGAELARPGASVKMSCKSS----- | 20-44 | 25 | 74 |
| | Kabat | QVQLQQSGAELARPGASVKMSCKSSGYTFT | 20-49 | 30 | 75 |
| | Contact | QVQLQQSGAELARPGASVKMSCKSSGYTF- | 20-48 | 29 | 76 |
| | IMGT | QVQLQQSGAELARPGASVKMSCKSS----- | 20-44 | 25 | 77 |
| CDR- | Chothia | GYTFTIY--- | 45-51 | 7 | 13 |
| H1 | AbM | GYTFTTYTIH | 45-54 | 10 | 14 |
| | Kabat | -----IYTIH | 50-54 | 5 | 15 |
| | Contact | ----TIYTIH | 49-54 | 6 | 16 |
| | IMGT | GYTFTIYT-- | 45-52 | 8 | 17 |
| HFR2 | Chothia | TIHWVKQRPGQGLEWIGYI | 52-70 | 19 | 78 |
| | AbM | ---WVKQRPGQGLEWIG-- | 55-68 | 14 | 79 |
| | Kabat | ---WVKQRPGQGLEWIG-- | 55-68 | 14 | 80 |
| | Contact | ---WVKQRPGQGLE----- | 55-65 | 11 | 81 |
| | IMGT | -IHWVKQRPGQGLEWIGY- | 53-69 | 17 | 82 |
| CDR- | Chothia | -----NPSSDY--------- | 71-76 | 6 | 18 |
| H2 | AbM | ---YINPSSDYTN------- | 69-78 | 10 | 19 |
| | Kabat | ---YINPSSDYTNYNQKFKD | 69-85 | 17 | 20 |
| | Contact | WIGYINPSSDYTN------- | 66-78 | 13 | 21 |
| | IMGT | ----INPSSDYT-------- | 70-77 | 8 | 22 |
| HFR3 | Chothia | TNYNQKFKDKATLTADKSSTTAYMQLSSLTSEDSAVYYCVR | 77-117 | 41 | 83 |
| | AbM | --YNQKFKDKATLTADKSSTTAYMQLSSLTSEDSAVYYCVR | 79-117 | 39 | 84 |
| | Kabat | ---------KATLTADKSSTTAYMQLSSLTSEDSAVYYCVR | 86-117 | 32 | 85 |
| | Contact | --YNQKFKDKATLTADKSSTTAYMQLSSLTSEDSAVYYC-- | 79-115 | 37 | 86 |
| | IMGT | -NYNQKFKDKATLTADKSSTTAYMQLSSLTSEDSAVYYC-- | 78-115 | 38 | 87 |
| CDR- | Chothia | --EGAYGNYDFAMDY | 118-130 | 13 | 23 |
| H3 | AbM | --EGAYGNYDFAMDY | 118-130 | 13 | 24 |
| | Kabat | --EGAYGNYDFAMDY | 118-130 | 13 | 25 |
| | Contact | VREGAYGNYDFAMD- | 116-129 | 14 | 26 |
| | IMGT | VREGAYGNYDFAMDY | 116-130 | 15 | 27 |
| HFR4 | Chothia | -WGQGTSVTVSS | 131-141 | 11 | 88 |
| | AbM | -WGQGTSVTVSS | 131-141 | 11 | 89 |
| | Kabat | -WGQGTSVTVSS | 131-141 | 11 | 90 |
| | Contact | YWGQGTSVTVSS | 130-141 | 12 | 91 |
| | IMGT | -WGQGTSVTVSS | 131-141 | 11 | 92 |

HFR = Heavy chain framework region

TABLE 5
Clone 3C3-1 Light Chain CDR1, 2 and 3 amino acid sequences.

| | | | | | SEQ |
|---|---|---|---|---|---|
| | | | | | ID |
| Region | Definition | Sequence Fragment | Residues | Length | NO: |
| LFR1 | Chothia | DVLMTQTPLSLPVSLGDQASISC------ | 20-42 | 23 | 93 |
| | AbM | DVLMTQTPLSLPVSLGDQASISC------ | 20-42 | 23 | 94 |
| | Kabat | DVLMTQTPLSLPVSLGDQASISC------ | 20-42 | 23 | 95 |
| | Contact | DVLMTQTPLSLPVSLGDQASISCRSSQNI | 20-48 | 29 | 96 |
| | IMGT | DVLMTQTPLSLPVSLGDQASISCRSS--- | 20-45 | 26 | 97 |
| CDR- | Chothia | RSSQNIVHNNGNTYLE-- | 43-58 | 16 | 28 |
| L1 | AbM | RSSQNIVHNNGNTYLE-- | 43-58 | 16 | 29 |
| | Kabat | RSSQNIVHNNGNTYLE-- | 43-58 | 16 | 30 |
| | Contact | ------VHNNGNTYLEWY | 49-60 | 12 | 31 |
| | IMGT | ---QNIVHNNGNTY---- | 46-56 | 11 | 32 |
| LFR2 | Chothia | --WYLQKPGQSPKLLIY | 59-73 | 15 | 98 |
| | AbM | --WYLQKPGQSPKLLIY | 59-73 | 15 | 99 |
| | Kabat | --WYLQKPGQSPKLLIY | 59-73 | 15 | 100 |
| | Contact | ----LQKPGQSPK---- | 61-69 | 9 | 101 |
| | IMGT | LEWYLQKPGQSPKLLIY | 57-73 | 17 | 102 |
| CDR- | Chothia | ----KVSNRFS | 74-80 | 7 | 33 |
| L2 | AbM | ----KVSNRFS | 74-80 | 7 | 34 |
| | Kabat | ----KVSNRFS | 74-80 | 7 | 35 |
| | Contact | LLIYKVSNRF- | 70-79 | 10 | 36 |
| | IMGT | ----KV----- | 74-75 | 2 | 37 |
| LFR3 | Chothia | -----GVPDRFSGSGSGTDFTLKISRVEAEDLGVYYC | 81-112 | 32 | 103 |
| | AbM | -----GVPDRFSGSGSGTDFTLKISRVEAEDLGVYYC | 81-112 | 32 | 104 |
| | Kabat | -----GVPDRFSGSGSGTDFTLKISRVEAEDLGVYYC | 81-112 | 32 | 105 |
| | Contact | ----SGVPDRFSGSGSGTDFTLKISRVEAEDLGVYYC | 80-112 | 33 | 106 |
| | IMGT | SNRFSGVPDRFSGSGSGTDFTLKISRVEAEDLGVYYC | 76-112 | 37 | 107 |
| CDR- | Chothia | FQTSHVPYT | 113-121 | 9 | 38 |
| L3 | AbM | FQTSHVPYT | 113-121 | 9 | 39 |
| | Kabat | FQTSHVPYT | 113-121 | 9 | 40 |
| | Contact | FQTSHVPY- | 113-120 | 8 | 41 |
| | IMGT | FQTSHVPYT | 113-121 | 9 | 42 |
| LFR4 | Chothia | -FGGGTKLEIK | 122-131 | 10 | 108 |
| | AbM | -FGGGTKLEIK | 122-131 | 10 | 109 |
| | Kabat | -FGGGTKLEIK | 122-131 | 10 | 110 |
| | Contact | TFGGGTKLEIK | 121-131 | 11 | 111 |
| | IMGT | -FGGGTKLEIK | 122-131 | 10 | 112 |

LFR = Light chain framework region

TABLE 6
Clone 2D4-1 Heavy Chain CDR1, 2 and 3 amino acid sequences.

| | | | | | SEQ |
|---|---|---|---|---|---|
| | | | | | ID |
| Region | Definition | Sequence Fragment | Residues | Length | NO: |
| HFR1 | Chothia | QVHLKESGPGLVAPSQSLSITCTVS----- | 20-44 | 25 | 113 |
| | AbM | QVHLKESGPGLVAPSQSLSITCTVS----- | 20-44 | 25 | 114 |
| | Kabat | QVHLKESGPGLVAPSQSLSITCTVSGFSLT | 20-49 | 30 | 115 |
| | Contact | QVHLKESGPGLVAPSQSLSITCTVSGFSL- | 20-48 | 29 | 116 |
| | IMGT | QVHLKESGPGLVAPSQSLSITCTVS----- | 20-44 | 25 | 117 |
| CDR- | Chothia | GFSLTGY--- | 45-51 | 7 | 43 |
| H1 | AbM | GFSLTGYGVN | 45-54 | 10 | 44 |
| | Kabat | -----GYGVN | 50-54 | 5 | 45 |
| | Contact | ----TGYGVN | 49-54 | 6 | 46 |
| | IMGT | GFSLTGYG-- | 45-52 | 8 | 47 |
| HFR2 | Chothia | GVNWIRQPPGKNLEWLGMI | 52-70 | 19 | 118 |
| | AbM | ---WIRQPPGKNLEWLG-- | 55-68 | 14 | 119 |
| | Kabat | ---WIRQPPGKNLEWLG-- | 55-68 | 14 | 120 |
| | Contact | ---WIRQPPGKNLE----- | 55-65 | 11 | 121 |
| | IMGT | -VNWIRQPPGKNLEWLGM- | 53-69 | 17 | 122 |
| CDR- | Chothia | -----WGDGS--------- | 71-75 | 5 | 48 |
| H2 | AbM | ---MIWGDGSTD------- | 69-77 | 9 | 49 |
| | Kabat | ---MIWGDGSTDYNSVLKS | 69-84 | 16 | 50 |
| | Contact | WLGMIWGDGSTD------- | 66-77 | 12 | 51 |
| | IMGT | ----IWGDGST-------- | 70-76 | 7 | 52 |
| HFR3 | Chothia | TDYNSVLKSRLSISKDNAKSQVFLEMNSLQTDDTANYYCAR | 76-116 | 41 | 123 |
| | AbM | --YNSVLKSRLSISKDNAKSQVFLEMNSLQTDDTANYYCAR | 78-116 | 39 | 124 |
| | Kabat | ---------RLSISKDNAKSQVFLEMNSLQTDDTANYYCAR | 85-116 | 32 | 125 |
| | Contact | --YNSVLKSRLSISKDNAKSQVFLEMNSLQTDDTANYYC-- | 78-114 | 37 | 126 |
| | IMGT | -DYNSVLKSRLSISKDNAKSQVFLEMNSLQTDDTANYYC-- | 77-114 | 38 | 127 |
| CDR- | Chothia | --DRGISYYYAMDY | 117-128 | 12 | 53 |
| H3 | AbM | --DRGISYYYAMDY | 117-128 | 12 | 54 |
| | Kabat | --DRGISYYYAMDY | 117-128 | 12 | 55 |
| | Contact | ARDRGISYYYAMD- | 115-127 | 13 | 56 |
| | IMGT | ARDRGISYYYAMDY | 115-128 | 14 | 57 |
| HFR4 | Chothia | -WGQGTSVTVSS | 129-139 | 11 | 128 |
| | AbM | -WGQGTSVTVSS | 129-139 | 11 | 129 |
| | Kabat | -WGQGTSVTVSS | 129-139 | 11 | 130 |
| | Contact | YWGQGTSVTVSS | 128-139 | 12 | 131 |
| | IMGT | -WGQGTSVTVSS | 129-139 | 11 | 132 |

TABLE 7
Clone 2D4-1 Light Chain CDR1, 2 and 3 amino acid sequences.

| | | | | | SEQ |
|---|---|---|---|---|---|
| | | | | | ID |
| Region | Definition | Sequence Fragment | Residues | Length | NO: |
| LFR1 | Chothia | DIVLTQSPASLAVSLGQRATISC------ | 21-43 | 23 | 133 |
| | AbM | DIVLTQSPASLAVSLGQRATISC------ | 21-43 | 23 | 134 |
| | Kabat | DIVLTQSPASLAVSLGQRATISC------ | 21-43 | 23 | 135 |
| | Contact | DIVLTQSPASLAVSLGQRATISCRASKSV | 21-49 | 29 | 136 |
| | IMGT | DIVLTQSPASLAVSLGQRATISCRAS--- | 21-46 | 26 | 137 |
| CDR- | Chothia | RASKSVSASGYSYLH-- | 44-58 | 15 | 58 |
| L1 | AbM | RASKSVSASGYSYLH-- | 44-58 | 15 | 59 |
| | Kabat | RASKSVSASGYSYLH-- | 44-58 | 15 | 60 |
| | Contact | ------SASGYSYLHWY | 50-60 | 11 | 61 |
| | IMGT | ---KSVSASGYSY---- | 47-56 | 10 | 62 |
| LFR2 | Chothia | --WYQQKPGQPPKLLIF | 59-73 | 15 | 138 |
| | AbM | --WYQQKPGQPPKLLIF | 59-73 | 15 | 139 |
| | Kabat | --WYQQKPGQPPKLLIF | 59-73 | 15 | 140 |
| | Contact | ----QQKPGQPPK---- | 61-69 | 9 | 141 |
| | IMGT | LHWYQQKPGQPPKLLIF | 57-73 | 17 | 142 |
| CDR- | Chothia | ----LAYNLGS | 74-80 | 7 | 63 |
| L2 | AbM | ----LAYNLGS | 74-80 | 7 | 64 |
| | Kabat | ----LAYNLGS | 74-80 | 7 | 65 |
| | Contact | LLIFLAYNLG- | 70-79 | 10 | 66 |
| | IMGT | ----LA----- | 74-75 | 2 | 67 |
| LFR3 | Chothia | -----GVPARFSGSGAGTDFTLNIHPVEEEDAATYYC | 81-112 | 32 | 143 |
| | AbM | -----GVPARFSGSGAGTDFTLNIHPVEEEDAATYYC | 81-112 | 32 | 144 |
| | Kabat | -----GVPARFSGSGAGTDFTLNIHPVEEEDAATYYC | 81-112 | 32 | 145 |
| | Contact | ----SGVPARFSGSGAGTDFTLNIHPVEEEDAATYYC | 80-112 | 33 | 146 |
| | IMGT | YNLGSGVPARFSGSGAGTDFTLNIHPVEEEDAATYYC | 76-112 | 37 | 147 |
| CDR- | Chothia | QHSREFPLT | 113-121 | 9 | 68 |
| L3 | AbM | QHSREFPLT | 113-121 | 9 | 69 |
| | Kabat | QHSREFPLT | 113-121 | 9 | 70 |
| | Contact | QHSREFPL- | 113-120 | 8 | 71 |
| | IMGT | QHSREFPLT | 113-121 | 9 | 72 |
| LFR4 | Chothia | -FGAGTKLELK | 122-131 | 10 | 148 |
| | AbM | -FGAGTKLELK | 122-131 | 10 | 149 |
| | Kabat | -FGAGTKLELK | 122-131 | 10 | 150 |
| | Contact | TFGAGTKLELK | 121-131 | 11 | 151 |
| | IMGT | -FGAGTKLELK | 122-131 | 10 | 152 |

### Results

### EphA3 on Glioma Cell Lines

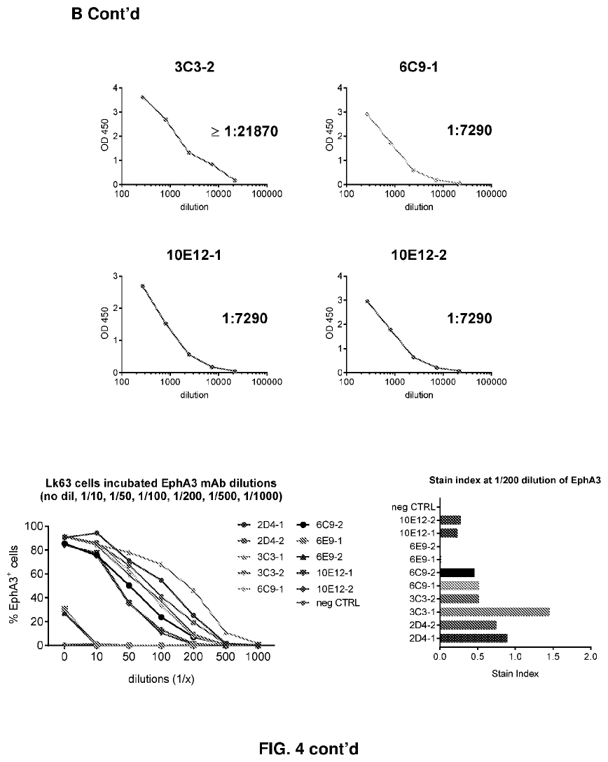

Clone 3C3-1 was used to screen glioma cell lines for EphA3 expression (FIG. 5). U87 cells were negative but D270 cells expressed a proportion of EphA3 positive and negative tumour cells. U251 cells are mostly EphA3 positive. These tumour cells lines will be valuable to test immunotherapeutic approaches on heterogeneous tumours (D270) but also evaluate particularly aggressive GBM (U251) with elevated EphA3 expression.

### EphA3-CAR T-Cell

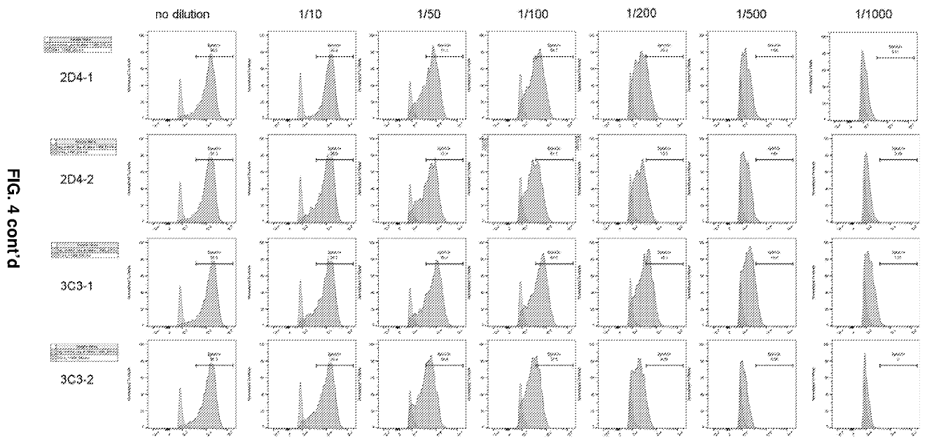

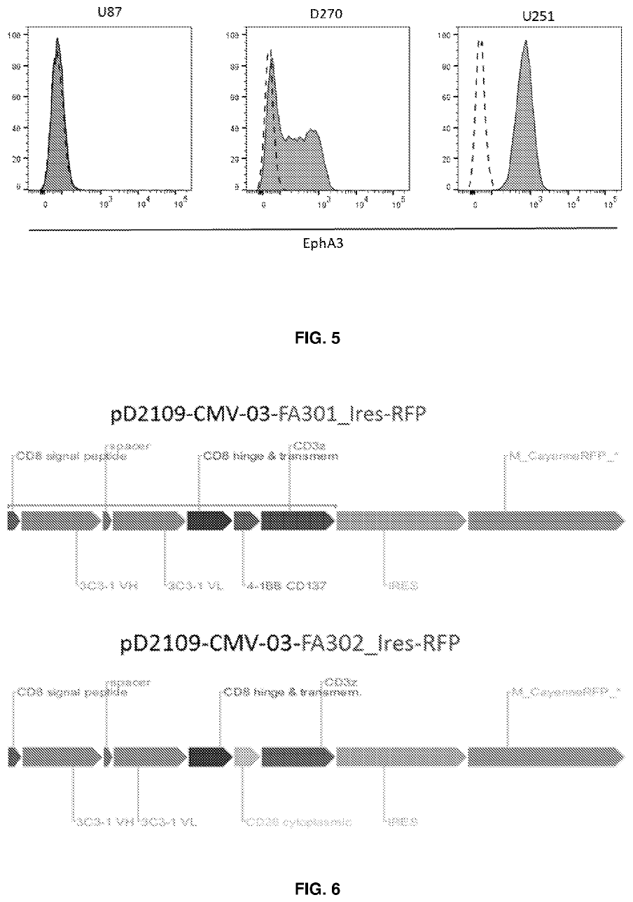

The single-chain variable fragment (scFv) consists of variable regions of heavy (V_{H}) and light (V_{L}) chains that are joined together by a flexible peptide linker. The scFv sequences of clones 3C3-1 and 2D4-1 were compared and the identity of alignment was <48%, meaning these are distinct sequences. These scFv sequences were used to generate lentiviral expression plasmids to create our second-generation CAR constructs. Briefly, we linked the individual coding sequences for the anti-EphA3 scFv to the hinge, CD8 transmembrane region, and the cytoplasmic regions of human 4-1 BB or CD28 with CD3-ζ (FIG. 6). Sequences were subcloned into the pD2109 (lentiviral backbone plasmid—ATUM) to generate the lentiviral expression plasmid. Lentiviral particles were produced via transfecting HEK293T human embryonic kidney cells. Cells were transfected with expression plasmids (FA301 or FA302) and pMDL, pREV and pVSV-G plasmids using Lipofectamine 2000. pD2109 was used as a control. Expression of the CAR sequences in 293T cells was confirmed by RT-PCR (FIG. 7). Viral supernatants were collected at 48 and 72 hours post transfection.

TABLE 8
Domains and sequences used to generate CAR. The variable regions (V_{H} ad V_{L})
of clones 3C3-1 and 2D4-1 were sequenced from monoclonal antibodies as
described in section 2.1. Other sequences were extracted from online databases
or supplied by ATUM ®

| | | SEQ |
|---|---|---|
| Domain | Sequence | ID NO. |
| 3C3-1 V_{H} | QVQLQQSGAELARPGASVKMSCKSSGYTFTIYTIHWVKQRPG | 153 |
| | QGLEWIGYINPSSDYTNYNQKFKDKATLTADKSSTTAYMQLS | |
| | SLTSEDSAVYYCVREGAYGNYDFAMDYWGQGTSVTVSS | |
| 3C3-1 V_{L} | DVLMTQTPLSLPVSLGDQASISCRSSQNIVHNNGNTYLEWYL | 154 |
| | QKPGQSPKLLIYKVSNRFSGVPDRFSGSGSGTDFTLKISRVEA | |
| | EDLGVYYCFQTSHVPYTFGGGTKLEIK | |
| 2D4-1 V_{H} | QVHLKESGPGLVAPSQSLSITCTVSGFSLTGYGVNWIRQPPG | 155 |
| | KNLEWLGMIWGDGSTDYNSVLKSRLSISKDNAKSQVFLEMNS | |
| | LQTDDTANYYCARDRGISYYYAMDYWGQGTSVTVSS | |
| 2D4-1 V_{L} | DIVLTQSPASLAVSLGQRATISCRASKSVSASGYSYLHWYQQ | 156 |
| | KPGQPPKLLIFLAYNLGSGVPARFSGSGAGTDFTLNIHPVEEE | |
| | DAATYYCQHSREFPLTFGAGTKLELK | |
| CD8 signal | MALPVTALLLPLALLLHAARP | 157 |
| peptide | | |
| AAH25715.1 | | |
| Spacer/linker | GGGGSGGGGSGGGGS | 158 |
| CD8 hinge & | TTTPAPRPPTPAPTIASQPLSLRPEACRPAAGGAVHTRGLDFA | 159 |
| transmembrane | CDIYIWAPLAGTCGVLLLSLVITLYC | |
| AAH25715.1 | | |
| 4-1BB | KRGRKKLLYIFKQPFMRPVQTTQEEDGCSCRFPEEEEGGCEL | 160 |
| NP_001552.2 | | |
| CD28 | RSKRSRLLHSDYMNMTPRRPGPTRKHYQPYAPPRDFAAYRS | 161 |
| cytoplasmic | | |
| NP_001230006.1 | | |
| CD3z_* | RVKFSRSADAPAYKQGQNQLYNELNLGRREEYDVLDKRRGR | 162 |
| NP_000725.1 | DPEMGGKPRRKNPQEGLYNELQKDKMAEAYSEIGMKGERRR | |
| | GKGHDGLYQGLSTATKDTYDALHMQALPPR* | |
| IRES (non | TAAGGTCTCTAAAATTCCGCCCCCCCCCTAACGTTACTGGC | 163 |
| coding) | CGAAGCCGCTTGGAATAAGGCCGGTGTGCGTTTGTCTATA | |
| | TGTTATTTTCCACCATATTGCCGTCTTTTGGCAATGTGAGG | |
| | GCCCGGAAACCTGGCCCTGTCTTCTTGACGAGCATTCCTA | |
| | GGGGTCTTTCCCCTCTCGCCAAAGGAATGCAAGGTCTGTT | |
| | GAATGTCGTGAAGGAAGCAGTTCCTCTGGAAGCTTCTTGAA | |
| | GACAAACAACGTCTGTAGCGACCCTTTGCAGGCAGCGGAA | |
| | CCCCCCACCTGGCGACAGGTGCCTCTGCGGCCAAAAGCC | |
| | ACGTGTATAAGATACACCTGCAAAGGCGGCACAACCCCAG | |
| | TGCCACGTTGTGAGTTGGATAGTTGTGGAAAGAGTCAAATG | |
| | GCTCTCCTCAAGCGTATTCAACAAGGGGCTGAAGGATGCC | |
| | CAGAAGGTACCCCATTGTATGGGATCTGATCTGGGGCCTC | |
| | GGTACACATGCTTTACATGTGTTTAGTCGAGGTTAAAAAAC | |
| | GTCTAGGCCCCCCGAACCACGGGGACGTGGTTTTCCTTTG | |
| | AAAAACACGATGATAATATGGCCACAACC | |
| M_ | MVSKGEELIKENMHMKLYMEGTVNNHHFKCTSEGEGKPYEG | 164 |
| CayenneRFP_* | TQTMRIKVVEGGPLPFAFDILATSFMYGSRTFIKYPKGIPDFFK | |
| | QSFPEGFTWERVTTYEDGGVVTVMQDTSLEDGCLVYNVKIR | |
| | GVNFPSNGPVMQKKTLGWEANTEMLYPADGGLEGRSDMAL | |
| | KLVGGGHLSCSFVTTYRSKKPAKNLKMPGIHAVDHRLERLEE | |
| | SDNEMFVVQREHAVARYCDLPSKLGHKLN* | |

### EphA3-CAR Expression in Jurkat Cell Line

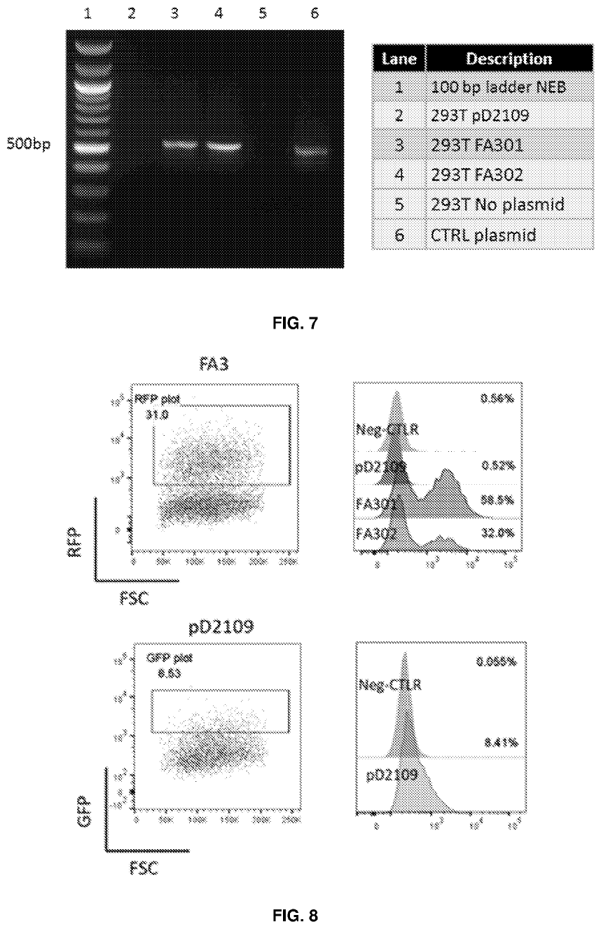

Jurkat cells are an immortalized human T-cell line and these were used to determine the titre of the lentiviral-containing supernatants. Since the CAR construct is in tandem with Ires_RFP, expression of RFP on the surface was used as a reporter of transduction. The transduction efficiency was therefore determined by transducing Jurkat cells and quantifying RFP expression. Transduction efficiency ranged from 32 to 58% (in 1×10⁶ cells) resulting in a titre range of 3.2 to 5.8×10⁵ IU/mL. The control pD2109_GFP lentiviral titre was 8×10⁴ IU/mL (FIG. 8).

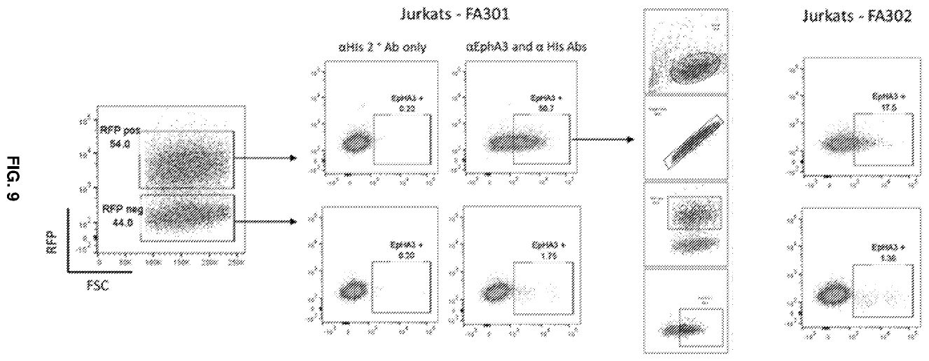

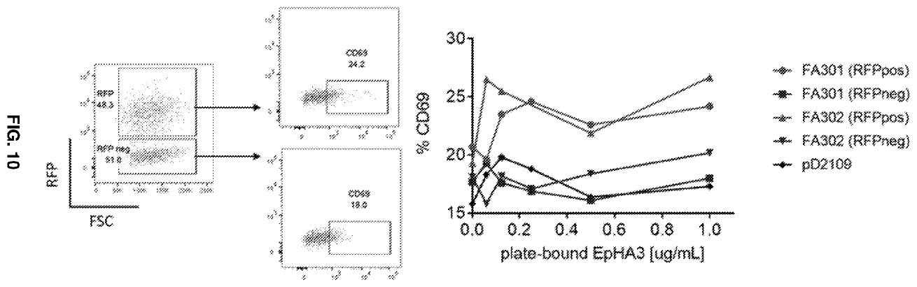

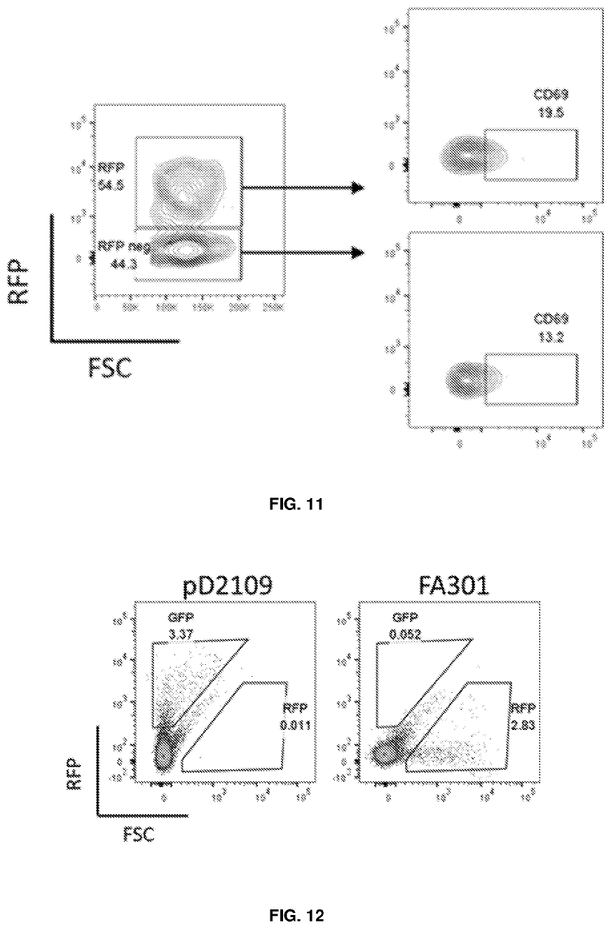

Both CAR and RFP sequences are headed by a CD8 leader sequence for surface membrane expression. Nonetheless, in order to confirm surface expression of the CAR and binding to target, cells were incubated with an EphA3-His protein and stained with an αHis-tag Ab. FACS results show that EphA3-CAR is expressed on the surface and binds EphA3 mostly in cells with high RFP expression (FIG. 9). D69 is an early activation marker in T-cells and is involved in proliferation and signal transduction. We used CD69 as a marker of EphA3-specific activation of Jurkat-CAR cells. Notwithstanding high levels of CD69 expression in RFP negative Jurkat cells, results show that modest activation occurs in cells that express the CAR construct (RFP positive cells) by interaction with membrane-bound EphA3 (FIG. 10) or by incubation with Lk63 cells (EphA3-positive tumour cell line) (FIG. 11). Although only a modest increase in CD69 expression was observed, the expression of activation markers in the Jurkat-CARs is a promising indicator of CAR functionality. It is speculated, therefore, that low level activation occurs in these cells since the multiplicity of infection (MOI) used for transduction was low resulting in low number of integrations (possibly only 1) per cell. We will address this in future by concentrating the lentiviruses in order to increase the MOI of transduction.

### PBMC Derived T-Cells for Generating an EphA3-CAR

PBMCs were harvested from peripheral blood by density gradient centrifugation within 24 hours of venesection. The PBMC fraction was removed, washed and counted. Polyclonal T-cells were generated by activation and expansion via CD3 and CD28 stimulation with T-cell TransAct™. CMV-specific T-cells were expanded from PBMCs using a previously described protocol.^{10,11} In brief, one-third of the PBMC were incubated with a custom pool of 26 T-cell peptide epitopes from multiple CMV antigens for one hour, washed then mixed with the remaining PBMC then seeded in flasks at density of between 2 and 5×10⁶ cells/cm².

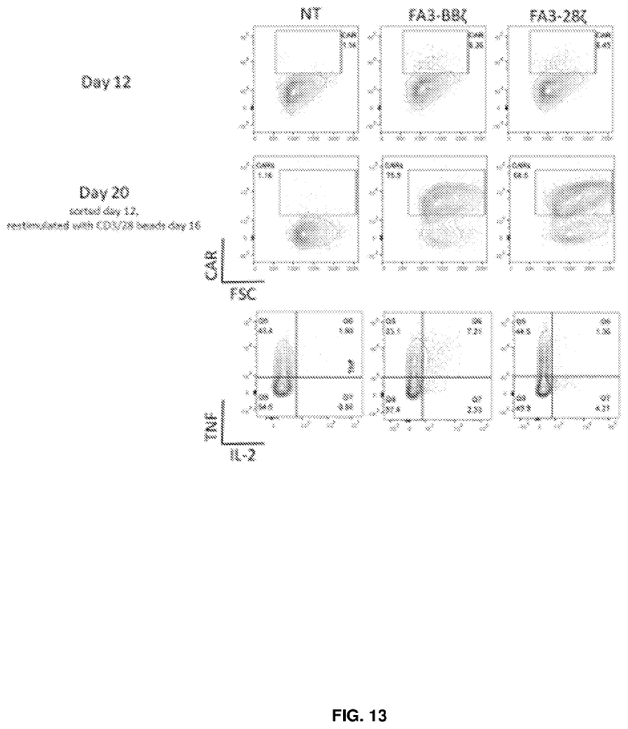

Cells were transduced on day 2 post-stimulation using pD2109 (GFP reporter) and FA301 (RFP reporter) lentiviruses. Cells were cultured in media containing recombinant IL-2 added every 2-3 days. FACS on day 3 post-transduction revealed a low transduction efficiency of both lentiviruses (FIG. 12).

### Conclusion

We have successfully generated EphA3-specific monoclonal antibodies which were used as the scFv in a CAR lentiviral construct. Jurkat-EphA3-CARs express the chimeric protein on the surface and upon recognition of EphA3, upregulate early activation markers. We further demonstrate that transduction of both Jurkat cells and CMV-specific T-cells with the Eph-A3-CARs is possible.

### Example 2

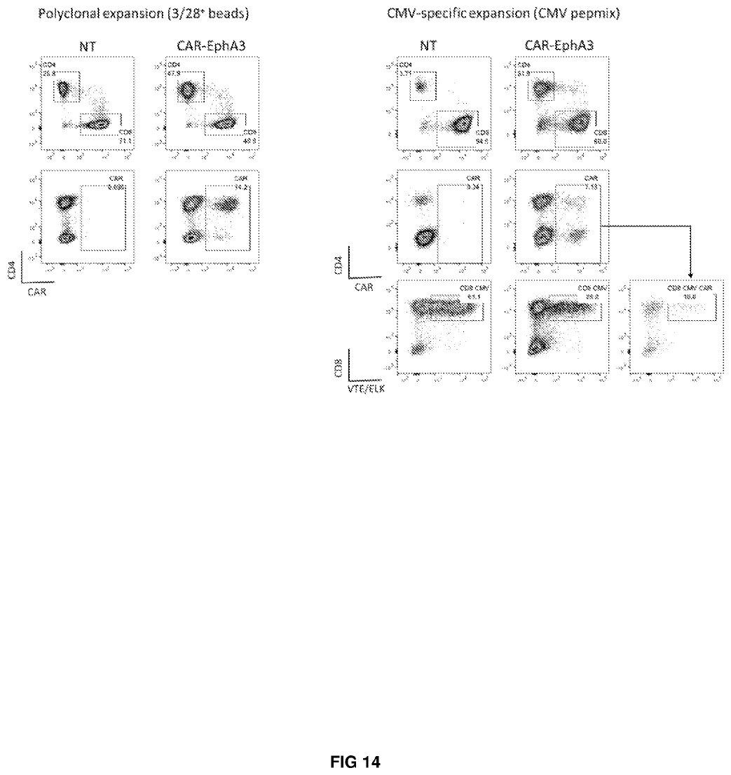

The IRES and RFP reporter sequences were removed from the constructs in order to reduce the size of the insert, with the goal of improving viral titre and T-cell transduction efficiency. These smaller constructs FA3-05-BBC and FA3-06-28 (were used to generate lentivirus as previously described, including an ultracentrifugation step at 10,500 rpm (SW 32 Ti rotor), 4 hours at 4° C. Polyclonal T cells were cultured and previously described, and transduced at day 2. CAR expressing T cells were detected by surface staining with anti-mouse IgG AF546 and cells analysed by flow cytometry. CAR transduction efficiency remained low at day 12. Cells were sorted for CAR+ expression and cultured up to day 20 (FIG. 13A).

We next assessed the in vitro functionality of these FA3-CARs. Transduced T-cells were stimulated overnight with LK63, an EphA3+ tumour cell line. Using a standard intracellular staining protocol we established that EphA3-CAR T-cells, with either 4-11BB (FA305) or CD28 (FA306) co-stimulation, undergo comparable target-induced cytokine secretion of TNF, a T cell activation and immune-modulating molecule (FIG. 13B).

The FA3-06-28ζ construct size was further reduced by using a custom pLV-Ef1a expression plasmid backbone from Biosettia. Subsequent studies were performed using T-cells transduced with lentivirus generated with this plasmid and will be referred to as CAR EpHA3 T-cells.

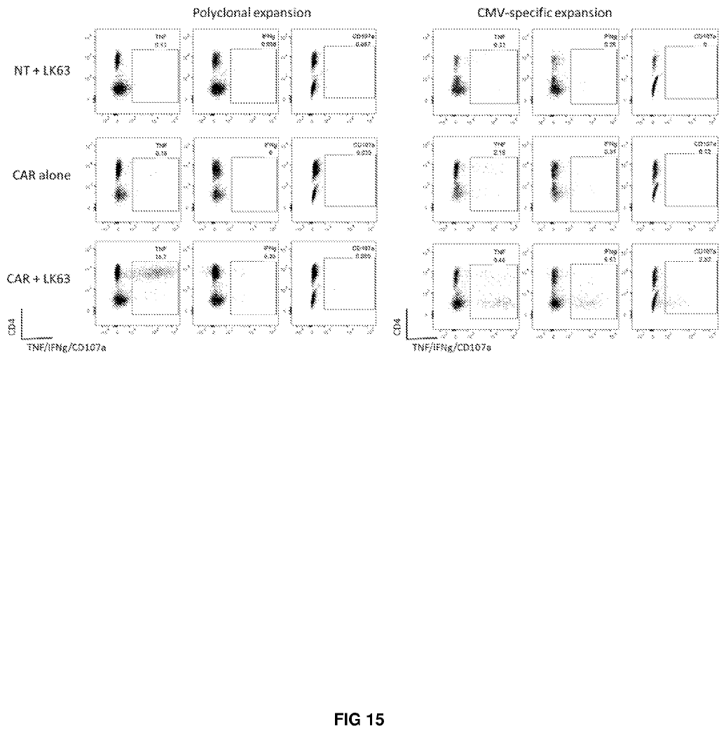

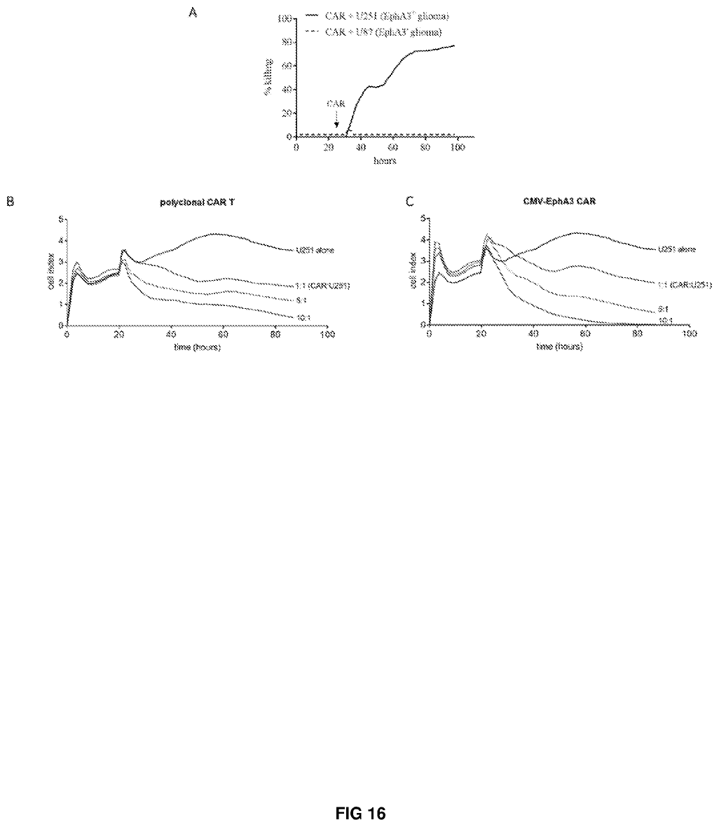

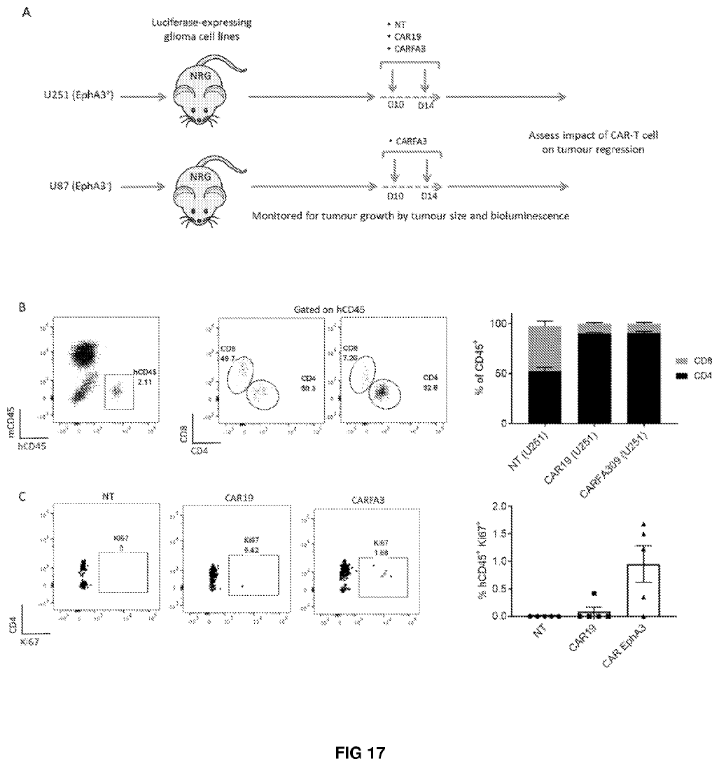

CAR EpHA3 lentivirus was used to transduce polyclonal T-cells (anti-CD3/28+-stimulated T-cells) and CMV-specific T-cells. CAR expression was determined as previously described and CMV-CAR specificity determined by FACS analysis using HLA complex—peptide tetramers for CMV (FIG. 14). In vitro functionality of the EphA3-CARs was determined as previously described. Transduced T-cells were stimulated with LK63 cells overnight. Using a standard intracellular staining protocol we established that EphA3-CAR T-cells undergo target-induced cytokine secretion of TNF. Following stimulation, CAR T-cells generated from the CMV-pepmix expressed multiple effector molecules including TNF, IFNγ and CD107a suggesting that these cells have greater killing potential (FIG. 15). To assess both specificity and killing capacity of EphA3-CAR, we performed a real-time cytotoxic assay (RTCA) using the xCELLigence. This assay measures target cell killing over a period of 100 hours. Glioma cell line U251, with endogenous EphA3 expression, was used as positive target alongside an EphA3 negative glioma cell, U87 as negative control. Previous studies by Day and colleagues have shown that the U251 EphA3+ glial cell line is responsive to anti-EphA3 (clone IIIA4) antibody in an orthotopic GBM model validating the use of these cells as a target.¹⁶ In the RTCA assay, the incubation of the target cells with EphA3-CAR T cells induced 80% cytolysis within 100 hours of treatment, and no killing of the EphA3 negative glioma cells was observed (FIG. 16A). EphA3 CAR killing of target cells was observed by a RTCA at 1:1, 5:1 and 10:1 effector to target ratios (FIG. 16B). To compare the killing potential of EphA3-CMV CAR T cells to EphA3 CARs we performed RTCA using T cell: target U251 ratios 1:1, 5:1 and 10:1 and observed efficient killing of the target cells, particularly at 10:1 and more evident with EphA3-CMV CAR T cells (FIG. 16C).

### Example 3

### EPHA3-CAR T-cells Exhibit a Potent Anti-Tumour Effect In Vivo

After showing that CAR EphA3 T-cells have significant in vitro cytotoxicity against glial cell lines, we next evaluated their therapeutic potential in vivo.

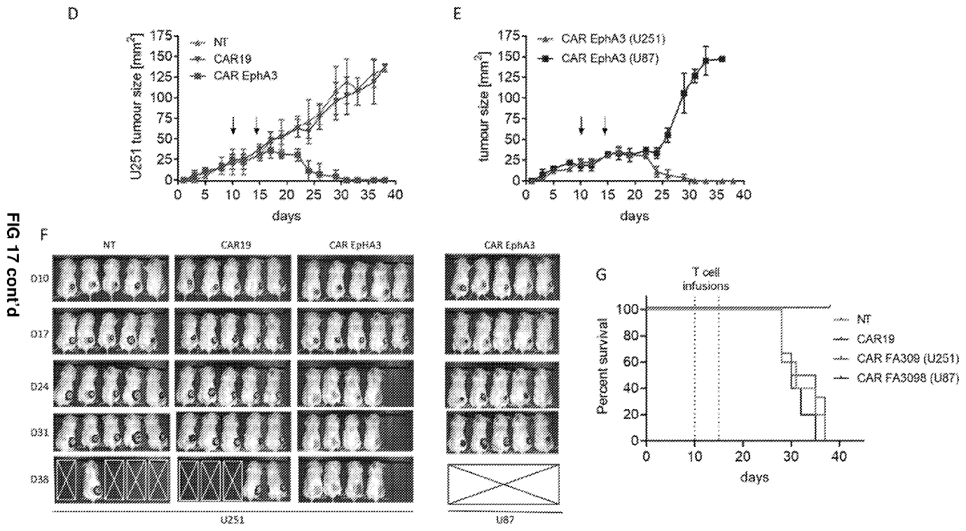

Immunodeficient NOD.Rag1 KO.IL2RycKO (NRG) mice were transplanted with luciferase-expressing glioma cell lines U251 (EphA3+) or U87 (EphA3−) subcutaneously in the flank (heterotopic model) (FIG. 17A). Tumour size was measured or determined by bioluminescence. At day 10, tumours had reached approximately 25 mm² so mice received the first of two intravenous injection of cells; EphA3-CAR, NT (non-transduced), or CAR19 (non-specific CAR T cells) T cells. Circulating hCD45 were detected at day 17 and were mostly CD4⁺CAR T cells (FIG. 17B). Furthermore, increased expression of Ki67 was observed in the U251 bearing mice which received EphA3-CAR T cells suggesting target induced proliferation of these CAR T cells in this treatment group (FIG. 17C).

Strikingly, the treatment with CAR EphA3 T-cells induced a complete response in mice transplanted with U251 (EphA3+) tumours and complete tumour clearance by day 30 (Figure D, & F). Mice which received non-transduced (NT) or non-specific T-cells (CAR19 T-cells) and U87 (EphA3−) bearing mice were not able to control tumour growth (FIG. 17D-G).

### Conclusion

These data clearly demonstrate that these CAR T-cells target EphA3 and mediate a potent anti-tumour activity. Treatment with EphA3 CAR T-cells results in tumour regression in a heterotopic xenograph GBM tumour model. These data support the use of EphA3 CAR T-cells as a new therapeutic for cancers, such as GBM.

### BIBLIOGRAPHY

1. Doubrovina, E. et al. Adoptive immunotherapy with unselected or EBV-specific T cells for biopsy-proven EBV+ lymphomas after allogeneic hematopoietic cell transplantation. Blood 119, 2644-2656 (2012).
2. Barrett, D. M., Grupp, S. A. & June, C. H. Chimeric Antigen Receptor- and TCR-Modified T Cells Enter Main Street and Wall Street. J. Immunol. 195, 755-61 (2015).
3. Day, B. W. et al. EphA3 Maintains Tumorigenicity and Is a Therapeutic Target in Glioblastoma Multiforme. Cancer Cell 23, 238-248 (2013).
4. Tang, X. X. et al. Implications of EPHB6, EFNB2, and EFNB3 expressions in human neuroblastoma. Proc. Natl. Acad. Sci. (2000) doi:10.1073/pnas.190123297.
5. Xi, H. Q., Wu, X. S., Wei, B. & Chen, L. Eph receptors and ephrins as targets for cancer therapy. J. Cell. Mol. Med. 16, 2894-2909 (2012).
6. Wykosky, J., Gibo, D. M. & Debinski, W. A novel, potent, and specific ephrinA1-based cytotoxin against EphA2 receptor expressing tumor cells. Mol. Cancer Ther. (2007) doi:10.1158/1535-7163.mct-07-0200.
7. Swords, R. T. et al. KB004, a Novel Non-Fucosylated Humaneered® Antibody, Targeting EphA3, Is Active and Well Tolerated in a Phase 1/11 Study of Advanced Hematologic Malignancies. Blood 124, (2014).
8. Razpotnik, R., Novak, N., Cǔrin Šerbec, V. & Rajcevic, U. Targeting Malignant Brain Tumors with Antibodies. Front. Immunol. 8, 1181 (2017).
9. Charmsaz, S. et al. EphA3 as a target for antibody immunotherapy in acute lymphoblastic leukemia. Leukemia 31, 1779-1787 (2017).
10. Smith, C. et al. Autologous Adoptive T-cell Therapy for Recurrent or Drug-resistant Cytomegalovirus Complications in Solid Organ Transplant Recipients: A Single-arm Open-label Phase I Clinical Trial. Clin. Infect. Dis. 68, 632-640 (2019).
11. Smith, C. et al. Autologous CMV-specific T cells are a safe adjuvant immunotherapy for primary glioblastoma multiforme. J. Clin. Invest. (2020) doi:10.1172/JCI1138649.
12. Day, B. W. et al. EphA3 Maintains Tumorigenicity and Is a Therapeutic Target in Glioblastoma Multiforme. Cancer Cell 23, 238-248 (2013).

1. (canceled)

2. An EphA3 binding agent comprising:
a. (i) a heavy chain immunoglobulin variable region (VH) polypeptide comprising a CDR 1 having an amino acid sequence at least 70% identical to any one of SEQ ID NOs:13-17; a CDR 2 having an amino acid sequence at least 70% identical to any one of SEQ ID NOs: 18: 22; and a CDR 3 having an amino acid sequence at least 70% identical to any one of SEQ ID NO: 23-27; and
(ii) a light chain immunoglobulin variable region (VL) polypeptide comprising a CDR 1 having an amino acid sequence at least 70% identical to any one of SEQ ID NOs: 28-32; a CDR 2 having an amino acid sequence at least 70% identical to any one of SEQ ID NOs: 33-37; and a CDR 3 having an amino acid sequence at least 70% identical to any one of SEQ ID NOs: 38-42: or
b. (i) a heavy chain immunoglobulin variable reaction (VH) polypeptide comprising a CDR 1 having an amino acid sequence at least 70% identical to any one of SEQ ID NOs: 43-47: a CDR 2 having an amino acid sequence at least 70% identical to any one of SEQ ID NOs: 48-52; and a CDR 3 having an amino acid sequence at least 70% identical to any one of SEQ ID NO:53-57; and
(ii) a light chain immunoglobulin variable region (VL) polypeptide comprising a CDR 1 having an amino acid sequence at least 70% identical to any one of SEQ ID NOs: 58-62; a CDR 2 having an amino acid sequence at least 70% identical to any one of SEQ ID NOs: 63-67; and a CDR 3 having an amino acid sequence at least 70% identical to any one of SEQ ID NOs: 68-72.

3. The EphA3 binding agent of claim 2, wherein:
a. (i) the VH polypeptide comprises an amino acid sequence set forth in SEQ ID NO:153 or an amino acid sequence at least 70% identical thereto; and/or
(ii) the VL polypeptide comprises an amino acid sequence set forth in SEQ ID NO:154 or an amino acid sequence at least 70% identical thereto;
b. (i) the VH polypeptide comprises an amino acid sequence set forth in SEQ ID NO: 155 or an amino acid sequence at least 70% identical thereto; and/or
(ii) the VL polypeptide comprises an amino acid sequence set forth in SEQ ID NO: 156 or an amino acid sequence at least 70% identical thereto.

4. (canceled)

5. (canceled)

6. The EphA3 binding agent of claim 2, wherein the EphA3 binding agent is an antibody or antibody fragment, optionally wherein the antibody or antibody fragment is a 3C3- or 2D-1 monoclonal antibody or fragment thereof.

7. (canceled)

8. The EphA3 binding agent of claim 2, wherein the EphA3 binding agent is a recombinant, human or humanized antibody or antibody fragment.

9. (canceled)

10. (canceled)

11. (canceled)

12. (canceled)

13. (canceled)

14. (canceled)

15. A chimeric antigen receptor (CAR) comprising an antigen binding domain including at least one CDR having an amino acid sequence set forth in SEQ ID NOs: 13-72 or an amino acid sequence at least 70% identical thereto, a transmembrane domain, and an intracellular T-cell signalling domain.

16. The CAR of claim 15, wherein the antigen binding domain comprises, consists or consists essentially of:
a (i) a heavy chain immunoglobulin variable region (VH) polypeptide comprising a CDR 1 having an amino acid sequence at least 70% identical to any one of SEQ ID NOs:13-17; a CDR 2 having an amino acid sequence at least 70% identical to any one of SEQ ID NOs: 18 to 22; and a CDR 3 having an amino acid sequence at least 70% identical to any one of SEQ ID NO: 23 to 27; and/or
(ii) a light chain immunoglobulin variable region (VL) polypeptide comprising a CDR 1 having an amino acid sequence at least 70% identical to any one of SEQ ID NOs: 28-32; a CDR 2 having an amino acid sequence at least 70% identical to any one of SEQ ID NOs: 33-37; and a CDR 3 having an amino acid sequence at least 70% identical to any one of SEQ ID NOs: 38-42; or
b. (i) a heavy chain immunoglobulin variable region (VH) polypeptide comprising a CDR 1 having an amino acid sequence at least 70% identical to any one of SEQ ID NOs:43-47; a CDR 2 having an amino acid sequence at least 70% identical to any one of SEQ ID NOs: 48 to 52: and a CDR 3 having an amino acid sequence at least 70% identical to any one of SEQ ID NO: 53 to 57; and/or
(ii) a light chain immunoglobulin variable region (VL) polypeptide comprising a CDR 1 having an amino acid sequence at least 70% identical to any one of SEQ ID NOs: 58-62: a CDR 2 having an amino acid sequence at least 70% identical to any one of SEQ ID NOs: 63-67; and a CDR 3 having an amino acid sequence at least 70% identical to any one of SEQ ID NOs: 68-72.

17. The CAR of claim 16, wherein:
a. (i) the VH polypeptide comprises an amino acid sequence set forth in SEQ ID NO:153 or an amino acid sequence at least 70% identical thereto; and/or
(ii) the VL polypeptide comprises an amino acid sequence set forth in SEQ ID NO:154 or an amino acid sequence at least 70% identical thereto; or
b. (i) the VH polypeptide comprises an amino acid sequence set forth in SEQ ID NO:155 or an amino acid sequence at least 70% identical thereto; and/or
(ii) the VL polypeptide comprises an amino acid sequence set forth in SEQ ID NO:156 or an amino acid sequence at least 70% identical thereto.

18. (canceled)

19. (canceled)

20. The CAR according to claim 15, wherein the antigen binding domain comprises a linker, such as the linker having an amino acid sequence set forth in SEQ ID NO: 158 or an amino acid sequence at least 70% identical thereto.

21. The CAR according to claim 15, further comprising a leader sequence, optionally wherein the leader sequence comprises, consists, or consists essentially of an amino acid sequence set forth in SEQ ID NO: 157 or an amino acid sequence at least 70% identical thereto.

22. (canceled)

23. The CAR according to claim 15, wherein the transmembrane domain comprises a CD8 transmembrane domain, optionally wherein the CD8 transmembrane domain comprises an amino acid sequence set forth in SEQ ID NO:159 or an amino acid sequence at least 70% identical thereto.

24. (canceled)

25. The CAR according to claim 15, wherein the intracellular signalling domain comprises a CD3 zeta intracellular signalling domain, optionally wherein the intracellular signalling domain comprises a CD3 zeta amino acid sequence set forth in SEQ ID NO:162 or an amino acid sequence at least 70% identical thereto.

26. (canceled)

27. The CAR according to claim 15, further comprising one or more co-stimulatory domains, such as a CD28 co-stimulatory domain having the amino acid sequence set forth in SEQ ID NO:161 or an amino acid sequence at least 70% identical thereto, and/or a CD137 co-stimulatory domain having the amino acid sequence set forth in SEQ ID NO:160 or an amino acid sequence at least 70% identical thereto.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A composition comprising the EphA3 binding agent according to claim 2 and a pharmaceutically acceptable carrier diluent or excipient.

40. (canceled)

41. (canceled)

42. A method of treating or preventing a cancer in a subject, said method including the step of administering a therapeutically effective amount of the EphA3 binding agent according to claim 2 to the subject to thereby treat or prevent the cancer in the subject.

43. (canceled)

44. (canceled)

45. (canceled)

46. (canceled)

47. (canceled)

48. (canceled)

49. A human T-cell expressing: (a) a T-cell receptor (TCR) that is activated by binding to a CMV antigen; and (b) a chimeric antigen receptor (CAR) comprising an antigen-binding domain that binds to an epitope on EphA3, optionally wherein the antigen-binding domain is a scFv comprising a heavy chain variable (VH) region and a tight chain variable (VL) region.

50. (canceled)

51. A T-cell that comprises (a) a T-cell receptor (TCR) that expresses a TCR that is specific for a CMV antigen; and (b) an antigen-binding molecule that binds to EphA3.

52. (canceled)

53. The T-cell of claim 51, wherein the antigen-binding molecule is a CAR.

54. The T-cell of claim 51, wherein the antigen-binding molecule is a scFv comprising a heavy chain variable (VH) region and a light chain variable (VL) region, optionally wherein the VH region has the amino acid sequence set forth in SEQ ID NO: 153, and wherein the VL region has an amino acid sequence that is set forth in SEQ ID NO: 154.

55. (canceled)

56. The T-cell of claim 51, wherein the CAR comprises one or both of:
(a) a transmembrane domain selected from a CD4, CD8, or CD28 transmembrane domain; and (b) a costimulatory domain selected from a 4-1 BB or CD28 co-stimulatory domain.

57. (canceled)

58. The T-cell of claim 51, wherein the CMV antigen comprises a peptide derived from one or more of pp50, pp65, IE-I, gB and gH, preferably wherein the CMV antigen comprises a peptide selected from the amino acid sequences set forth in SEQ ID NO: 181-211.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)


SEQUENCE LISTING (216 sequences)

SEQ ID NO: 1 - DNA, 15 bp, Artificial Sequence
  Heavy Chain CDR1 nucleotide sequence
atctacacgatacac

SEQ ID NO: 2 - DNA, 51 bp, Artificial Sequence
  Heavy Chain CDR2 nucleotide sequence
tacattaatcctagcagtgattatactaattacaatcagaagttcaaggac

SEQ ID NO: 3 - DNA, 39 bp, Artificial Sequence
  Heavy Chain CDR3 nucleotide sequence
gaaggggcgtatggtaactacgactttgctatggactac

SEQ ID NO: 4 - DNA, 48 bp, Artificial Sequence
  Light Chain CDR1 nucleotide sequence
agatctagtcagaacattgtccataataatggaaacacctatttagaa

SEQ ID NO: 5 - DNA, 21 bp, Artificial Sequence
  Light Chain CDR2 nucleotide sequence
aaagtttccaaccgattttct

SEQ ID NO: 6 - DNA, 27 bp, Artificial Sequence
  Light Chain CDR3 nucleotide sequence
tttcaaacttcacatgttccgtacacg

SEQ ID NO: 7 - DNA, 15 bp, Artificial Sequence
  Heavy Chain CDR1 nucleotide sequence
ggctatggtgtaaac

SEQ ID NO: 8 - DNA, 48 bp, Artificial Sequence
  Heavy Chain CDR2 nucleotide sequence
atgatatggggtgatggaagcacagactataattcagttctcaaatcc

SEQ ID NO: 9 - DNA, 36 bp, Artificial Sequence
  Heavy Chain CDR3 nucleotide sequence
gatcggggtattagttattactatgctatggactac

SEQ ID NO: 10 - DNA, 45 bp, Artificial Sequence
  Light Chain CDR1 nucleotide sequence
agggccagtaaaagtgtcagtgcatctggctatagttatttgcac

SEQ ID NO: 11 - DNA, 21 bp, Artificial Sequence
  Light Chain CDR2 nucleotide sequence
cttgcatacaacctaggatct

SEQ ID NO: 12 - DNA, 27 bp, Artificial Sequence
  Light Chain CDR3 nucleotide sequence
cagcacagtagggagtttccgctcacg

SEQ ID NO: 13 - protein, 7 aa, Artificial Sequence
  Heavy Chain CDR1 amino acid sequence (Chothia)
GYTFTIY

SEQ ID NO: 14 - protein, 10 aa, Artificial Sequence
  Heavy Chain CDR1 amino acid sequence (AbM)
GYTFTIYTIH

SEQ ID NO: 15 - protein, 5 aa, Artificial Sequence
  Heavy Chain CDR1 amino acid sequence (Kabat)
IYTIH

SEQ ID NO: 16 - protein, 6 aa, Artificial Sequence
  Heavy Chain CDR1 amino acid sequence (Contact)
TIYTIH

SEQ ID NO: 17 - protein, 8 aa, Artificial Sequence
  Heavy Chain CDR1 amino acid sequence (IMGT)
GYTFTIYT

SEQ ID NO: 18 - protein, 6 aa, Artificial Sequence
  Heavy Chain CDR2 amino acid sequence (Chothia)
NPSSDY

SEQ ID NO: 19 - protein, 10 aa, Artificial Sequence
  Heavy Chain CDR2 amino acid sequence (AbM)
YINPSSDYTN

SEQ ID NO: 20 - protein, 17 aa, Artificial Sequence
  Heavy Chain CDR2 amino acid sequence (Kabat)
YINPSSDYTNYNQKFKD

SEQ ID NO: 21 - protein, 13 aa, Artificial Sequence
  Synthetic Construct
WIGYINPSSDYTN

SEQ ID NO: 22 - protein, 8 aa, Artificial Sequence
  Heavy Chain CDR2 amino acid sequence (IMGT)
INPSSDYT

SEQ ID NO: 23 - protein, 13 aa, Artificial Sequence
  Heavy Chain CDR3 amino acid sequence (Chothia)
EGAYGNYDFAMDY

SEQ ID NO: 24 - protein, 13 aa, Artificial Sequence
  Heavy Chain CDR3 amino acid sequence (AbM)
EGAYGNYDFAMDY

SEQ ID NO: 25 - protein, 13 aa, Artificial Sequence
  Heavy Chain CDR3 amino acid sequence (Kabat)
EGAYGNYDFAMDY

SEQ ID NO: 26 - protein, 14 aa, Artificial Sequence
  Heavy Chain CDR3 amino acid sequence (Contact)
VREGAYGNYDFAMD

SEQ ID NO: 27 - protein, 15 aa, Artificial Sequence
  Heavy Chain CDR3 amino acid sequence (IMGT)
VREGAYGNYDFAMDY

SEQ ID NO: 28 - protein, 16 aa, Artificial Sequence
  Light Chain CDR1 amino acid sequence (Chothia)
RSSQNIVHNNGNTYLE

SEQ ID NO: 29 - protein, 16 aa, Artificial Sequence
  Light Chain CDR1 amino acid sequence (AbM)
RSSQNIVHNNGNTYLE

SEQ ID NO: 30 - protein, 16 aa, Artificial Sequence
  Light Chain CDR1 amino acid sequence (Kabat)
RSSQNIVHNNGNTYLE

SEQ ID NO: 31 - protein, 12 aa, Artificial Sequence
  Light Chain CDR1 amino acid sequence (Contact)
VHNNGNTYLEWY

SEQ ID NO: 32 - protein, 11 aa, Artificial Sequence
  Light Chain CDR1 amino acid sequence (IMGT)
QNIVHNNGNTY

SEQ ID NO: 33 - protein, 7 aa, Artificial Sequence
  Light Chain CDR2 amino acid sequence (Chothia)
KVSNRFS

SEQ ID NO: 34 - protein, 7 aa, Artificial Sequence
  Light Chain CDR2 amino acid sequence (AbM)
KVSNRFS

SEQ ID NO: 35 - protein, 7 aa, Artificial Sequence
  Light Chain CDR2 amino acid sequence (Kabat)
KVSNRFS

SEQ ID NO: 36 - protein, 10 aa, Artificial Sequence
  Light Chain CDR2 amino acid sequence (Contact)
LLIYKVSNRF

SEQ ID NO: 37 - protein, 2 aa, Artificial Sequence
  Light Chain CDR2 amino acid sequence (IMGT)
KV

SEQ ID NO: 38 - protein, 9 aa, Artificial Sequence
  Light Chain CDR3 amino acid sequence (Chothia)
FQTSHVPYT

SEQ ID NO: 39 - protein, 9 aa, Artificial Sequence
  Light Chain CDR3 amino acid sequence (AbM)
FQTSHVPYT

SEQ ID NO: 40 - protein, 9 aa, Artificial Sequence
  Light Chain CDR3 amino acid sequence (Kabat)
FQTSHVPYT

SEQ ID NO: 41 - protein, 8 aa, Artificial Sequence
  Clone 3C3-1 Light Chain CDR3 amino acid sequence
FQTSHVPY

SEQ ID NO: 42 - protein, 9 aa, Artificial Sequence
  Synthetic Construct
FQTSHVPYT

SEQ ID NO: 43 - protein, 7 aa, Artificial Sequence
  Synthetic Construct
GFSLTGY

SEQ ID NO: 44 - protein, 10 aa, Artificial Sequence
  Synthetic Construct
GFSLTGYGVN

SEQ ID NO: 45 - protein, 5 aa, Artificial Sequence
  Synthetic Construct
GYGVN

SEQ ID NO: 46 - protein, 6 aa, Artificial Sequence
  Synthetic Construct
TGYGVN

SEQ ID NO: 47 - protein, 8 aa, Artificial Sequence
  Synthetic Construct
GFSLTGYG

SEQ ID NO: 48 - protein, 5 aa, Artificial Sequence
  Synthetic Construct
WGDGS

SEQ ID NO: 49 - protein, 9 aa, Artificial Sequence
  Synthetic Construct
MIWGDGSTD

SEQ ID NO: 50 - protein, 16 aa, Artificial Sequence
  Synthetic Construct
MIWGDGSTDYNSVLKS

SEQ ID NO: 51 - protein, 12 aa, Artificial Sequence
  Synthetic Construct
WLGMIWGDGSTD

SEQ ID NO: 52 - protein, 7 aa, Artificial Sequence
  Synthetic Construct
IWGDGST

SEQ ID NO: 53 - protein, 12 aa, Artificial Sequence
  Synthetic Construct
DRGISYYYAMDY

SEQ ID NO: 54 - protein, 12 aa, Artificial Sequence
  Synthetic Construct
DRGISYYYAMDY

SEQ ID NO: 55 - protein, 12 aa, Artificial Sequence
  Synthetic Construct
DRGISYYYAMDY

SEQ ID NO: 56 - protein, 13 aa, Artificial Sequence
  Synthetic Construct
ARDRGISYYYAMD

SEQ ID NO: 57 - protein, 14 aa, Artificial Sequence
  Synthetic Construct
ARDRGISYYYAMDY

SEQ ID NO: 58 - protein, 15 aa, Artificial Sequence
  Synthetic Construct
RASKSVSASGYSYLH

SEQ ID NO: 59 - protein, 15 aa, Artificial Sequence
  Synthetic Construct
RASKSVSASGYSYLH

SEQ ID NO: 60 - protein, 15 aa, Artificial Sequence
  Synthetic Construct
RASKSVSASGYSYLH

SEQ ID NO: 61 - protein, 11 aa, Artificial Sequence
  Synthetic Construct
SASGYSYLHWY

SEQ ID NO: 62 - protein, 10 aa, Artificial Sequence
  Synthetic Construct
KSVSASGYSY

SEQ ID NO: 63 - protein, 7 aa, Artificial Sequence
  Synthetic Construct
LAYNLGS

SEQ ID NO: 64 - protein, 7 aa, Artificial Sequence
  Synthetic Construct
LAYNLGS

SEQ ID NO: 65 - protein, 7 aa, Artificial Sequence
  Synthetic Construct
LAYNLGS

SEQ ID NO: 66 - protein, 10 aa, Artificial Sequence
  Synthetic Construct
LLIFLAYNLG

SEQ ID NO: 67 - protein, 2 aa, Artificial Sequence
  Synthetic Construct
LA

SEQ ID NO: 68 - protein, 9 aa, Artificial Sequence
  Synthetic Construct
QHSREFPLT

SEQ ID NO: 69 - protein, 9 aa, Artificial Sequence
  Synthetic Construct
QHSREFPLT

SEQ ID NO: 70 - protein, 9 aa, Artificial Sequence
  Synthetic Construct
QHSREFPLT

SEQ ID NO: 71 - protein, 8 aa, Artificial Sequence
  Synthetic Construct
QHSREFPL

SEQ ID NO: 72 - protein, 9 aa, Artificial Sequence
  Synthetic Construct
QHSREFPLT

SEQ ID NO: 73 - protein, 25 aa, Artificial Sequence
  Synthetic Construct
QVQLQQSGAELARPGASVKMSCKSS

SEQ ID NO: 74 - protein, 25 aa, Artificial Sequence
  Synthetic Construct
QVQLQQSGAELARPGASVKMSCKSS

SEQ ID NO: 75 - protein, 30 aa, Artificial Sequence
  Synthetic Construct
QVQLQQSGAELARPGASVKMSCKSSGYTFT

SEQ ID NO: 76 - protein, 29 aa, Artificial Sequence
  Synthetic Construct
QVQLQQSGAELARPGASVKMSCKSSGYTF

SEQ ID NO: 77 - protein, 25 aa, Artificial Sequence
  Synthetic Construct
QVQLQQSGAELARPGASVKMSCKSS

SEQ ID NO: 78 - protein, 19 aa, Artificial Sequence
  Synthetic Construct
TIHWVKQRPGQGLEWIGYI

SEQ ID NO: 79 - protein, 14 aa, Artificial Sequence
  Synthetic Construct
WVKQRPGQGLEWIG

SEQ ID NO: 80 - protein, 14 aa, Artificial Sequence
  Synthetic Construct
WVKQRPGQGLEWIG

SEQ ID NO: 81 - protein, 11 aa, Artificial Sequence
  Synthetic Construct
WVKQRPGQGLE

SEQ ID NO: 82 - protein, 17 aa, Artificial Sequence
  Synthetic Construct
IHWVKQRPGQGLEWIGY

SEQ ID NO: 83 - protein, 41 aa, Artificial Sequence
  Synthetic Construct
TNYNQKFKDKATLTADKSSTTAYMQLSSLTSEDSAVYYCVR

SEQ ID NO: 84 - protein, 39 aa, Artificial Sequence
  Synthetic Construct
YNQKFKDKATLTADKSSTTAYMQLSSLTSEDSAVYYCVR

SEQ ID NO: 85 - protein, 32 aa, Artificial Sequence
  Synthetic Construct
KATLTADKSSTTAYMQLSSLTSEDSAVYYCVR

SEQ ID NO: 86 - protein, 37 aa, Artificial Sequence
  Synthetic Construct
YNQKFKDKATLTADKSSTTAYMQLSSLTSEDSAVYYC

SEQ ID NO: 87 - protein, 38 aa, Artificial Sequence
  Synthetic Construct
NYNQKFKDKATLTADKSSTTAYMQLSSLTSEDSAVYYC

SEQ ID NO: 88 - protein, 11 aa, Artificial Sequence
  Synthetic Construct
WGQGTSVTVSS

SEQ ID NO: 89 - protein, 11 aa, Artificial Sequence
  Synthetic Construct
WGQGTSVTVSS

SEQ ID NO: 90 - protein, 11 aa, Artificial Sequence
  Synthetic Construct
WGQGTSVTVSS

SEQ ID NO: 91 - protein, 12 aa, Artificial Sequence
  Synthetic Construct
YWGQGTSVTVSS

SEQ ID NO: 92 - protein, 11 aa, Artificial Sequence
  Synthetic Construct
WGQGTSVTVSS

SEQ ID NO: 93 - protein, 23 aa, Artificial Sequence
  Synthetic Construct
DVLMTQTPLSLPVSLGDQASISC

SEQ ID NO: 94 - protein, 23 aa, Artificial Sequence
  Synthetic Construct
DVLMTQTPLSLPVSLGDQASISC

SEQ ID NO: 95 - protein, 23 aa, Artificial Sequence
  Synthetic Construct
DVLMTQTPLSLPVSLGDQASISC

SEQ ID NO: 96 - protein, 29 aa, Artificial Sequence
  Synthetic Construct
DVLMTQTPLSLPVSLGDQASISCRSSQNI

SEQ ID NO: 97 - protein, 26 aa, Artificial Sequence
  Synthetic Construct
DVLMTQTPLSLPVSLGDQASISCRSS

SEQ ID NO: 98 - protein, 15 aa, Artificial Sequence
  Synthetic Construct
WYLQKPGQSPKLLIY

SEQ ID NO: 99 - protein, 15 aa, Artificial Sequence
  Synthetic Construct
WYLQKPGQSPKLLIY

SEQ ID NO: 100 - protein, 15 aa, Artificial Sequence
  Synthetic Construct
WYLQKPGQSPKLLIY

SEQ ID NO: 101 - protein, 9 aa, Artificial Sequence
  Synthetic Construct
LQKPGQSPK

SEQ ID NO: 102 - protein, 17 aa, Artificial Sequence
  Synthetic Construct
LEWYLQKPGQSPKLLIY

SEQ ID NO: 103 - protein, 32 aa, Artificial Sequence
  Synthetic Construct
GVPDRFSGSGSGTDFTLKISRVEAEDLGVYYC

SEQ ID NO: 104 - protein, 32 aa, Artificial Sequence; identical to SEQ ID NO: 103
  Synthetic Construct

SEQ ID NO: 105 - protein, 32 aa, Artificial Sequence; identical to SEQ ID NO: 103
  Synthetic Construct

SEQ ID NO: 106 - protein, 33 aa, Artificial Sequence
  Synthetic Construct
SGVPDRFSGSGSGTDFTLKISRVEAEDLGVYYC

SEQ ID NO: 107 - protein, 37 aa, Artificial Sequence
  Synthetic Construct
SNRFSGVPDRFSGSGSGTDFTLKISRVEAEDLGVYYC

SEQ ID NO: 108 - protein, 10 aa, Artificial Sequence
  Synthetic Construct
FGGGTKLEIK

SEQ ID NO: 109 - protein, 10 aa, Artificial Sequence
  Synthetic Construct
FGGGTKLEIK

SEQ ID NO: 110 - protein, 10 aa, Artificial Sequence
  Synthetic Construct
FGGGTKLEIK

SEQ ID NO: 111 - protein, 11 aa, Artificial Sequence
  Synthetic Construct
TFGGGTKLEIK

SEQ ID NO: 112 - protein, 10 aa, Artificial Sequence
  Synthetic Construct
FGGGTKLEIK

SEQ ID NO: 113 - protein, 25 aa, Artificial Sequence
  Synthetic Construct
QVHLKESGPGLVAPSQSLSITCTVS

SEQ ID NO: 114 - protein, 25 aa, Artificial Sequence
  Synthetic Construct
QVHLKESGPGLVAPSQSLSITCTVS

SEQ ID NO: 115 - protein, 30 aa, Artificial Sequence
  Synthetic Construct
QVHLKESGPGLVAPSQSLSITCTVSGFSLT

SEQ ID NO: 116 - protein, 29 aa, Artificial Sequence
  Synthetic Construct
QVHLKESGPGLVAPSQSLSITCTVSGFSL

SEQ ID NO: 117 - protein, 25 aa, Artificial Sequence
  Synthetic Construct
QVHLKESGPGLVAPSQSLSITCTVS

SEQ ID NO: 118 - protein, 19 aa, Artificial Sequence
  Synthetic Construct
GVNWIRQPPGKNLEWLGMI

SEQ ID NO: 119 - protein, 14 aa, Artificial Sequence
  Synthetic Construct
WIRQPPGKNLEWLG

SEQ ID NO: 120 - protein, 14 aa, Artificial Sequence
  Synthetic Construct
WIRQPPGKNLEWLG

SEQ ID NO: 121 - protein, 11 aa, Artificial Sequence
  Synthetic Construct
WIRQPPGKNLE

SEQ ID NO: 122 - protein, 17 aa, Artificial Sequence
  Synthetic Construct
VNWIRQPPGKNLEWLGM

SEQ ID NO: 123 - protein, 41 aa, Artificial Sequence
  Synthetic Construct
TDYNSVLKSRLSISKDNAKSQVFLEMNSLQTDDTANYYCAR

SEQ ID NO: 124 - protein, 41 aa, Artificial Sequence; identical to SEQ ID NO: 123
  Synthetic Construct

SEQ ID NO: 125 - protein, 32 aa, Artificial Sequence
  Synthetic Construct
RLSISKDNAKSQVFLEMNSLQTDDTANYYCAR

SEQ ID NO: 126 - protein, 37 aa, Artificial Sequence
  Synthetic Construct
YNSVLKSRLSISKDNAKSQVFLEMNSLQTDDTANYYC

SEQ ID NO: 127 - protein, 38 aa, Artificial Sequence
  Synthetic Construct
DYNSVLKSRLSISKDNAKSQVFLEMNSLQTDDTANYYC

SEQ ID NO: 128 - protein, 11 aa, Artificial Sequence
  Synthetic Construct
WGQGTSVTVSS

SEQ ID NO: 129 - protein, 11 aa, Artificial Sequence
  Synthetic Construct
WGQGTSVTVSS

SEQ ID NO: 130 - protein, 11 aa, Artificial Sequence
  Synthetic Construct
WGQGTSVTVSS

SEQ ID NO: 131 - protein, 12 aa, Artificial Sequence
  Synthetic Construct
YWGQGTSVTVSS

SEQ ID NO: 132 - protein, 11 aa, Artificial Sequence
  Synthetic Construct
WGQGTSVTVSS

SEQ ID NO: 133 - protein, 23 aa, Artificial Sequence
  Synthetic Construct
DIVLTQSPASLAVSLGQRATISC

SEQ ID NO: 134 - protein, 23 aa, Artificial Sequence
  Synthetic Construct
DIVLTQSPASLAVSLGQRATISC

SEQ ID NO: 135 - protein, 23 aa, Artificial Sequence
  Synthetic Construct
DIVLTQSPASLAVSLGQRATISC

SEQ ID NO: 136 - protein, 29 aa, Artificial Sequence
  Synthetic Construct
DIVLTQSPASLAVSLGQRATISCRASKSV

SEQ ID NO: 137 - protein, 26 aa, Artificial Sequence
  Synthetic Construct
DIVLTQSPASLAVSLGQRATISCRAS

SEQ ID NO: 138 - protein, 15 aa, Artificial Sequence
  Synthetic Construct
WYQQKPGQPPKLLIF

SEQ ID NO: 139 - protein, 15 aa, Artificial Sequence
  Synthetic Construct
WYQQKPGQPPKLLIF

SEQ ID NO: 140 - protein, 15 aa, Artificial Sequence
  Synthetic Construct
WYQQKPGQPPKLLIF

SEQ ID NO: 141 - protein, 9 aa, Artificial Sequence
  Synthetic Construct
QQKPGQPPK

SEQ ID NO: 142 - protein, 17 aa, Artificial Sequence
  Synthetic Construct
LHWYQQKPGQPPKLLIF

SEQ ID NO: 143 - protein, 32 aa, Artificial Sequence
  Synthetic Construct
GVPARFSGSGAGTDFTLNIHPVEEEDAATYYC

SEQ ID NO: 144 - protein, 32 aa, Artificial Sequence; identical to SEQ ID NO: 143
  Synthetic Construct

SEQ ID NO: 145 - protein, 32 aa, Artificial Sequence; identical to SEQ ID NO: 143
  Synthetic Construct

SEQ ID NO: 146 - protein, 33 aa, Artificial Sequence
  Synthetic Construct
SGVPARFSGSGAGTDFTLNIHPVEEEDAATYYC

SEQ ID NO: 147 - protein, 37 aa, Artificial Sequence
  Synthetic Construct
YNLGSGVPARFSGSGAGTDFTLNIHPVEEEDAATYYC

SEQ ID NO: 148 - protein, 10 aa, Artificial Sequence
  Synthetic Construct
FGAGTKLELK

SEQ ID NO: 149 - protein, 10 aa, Artificial Sequence
  Synthetic Construct
FGAGTKLELK

SEQ ID NO: 150 - protein, 10 aa, Artificial Sequence
  Synthetic Construct
FGAGTKLELK

SEQ ID NO: 151 - protein, 11 aa, Artificial Sequence
  Synthetic Construct
TFGAGTKLELK

SEQ ID NO: 152 - protein, 10 aa, Artificial Sequence
  Synthetic Construct
FGAGTKLELK

SEQ ID NO: 153 - protein, 122 aa, Artificial Sequence
  Synthetic Construct
QVQLQQSGAELARPGASVKMSCKSSGYTFTIYTIHWVKQRPGQGLEWIGYINPSSDYTNYNQKFKDKATLTADKSSTTAYMQLSSLTSEDSAVYYCVREGAYGNYDFAMDYWGQGTSVTVSS

SEQ ID NO: 154 - protein, 112 aa, Artificial Sequence
  Synthetic Construct
DVLMTQTPLSLPVSLGDQASISCRSSQNIVHNNGNTYLEWYLQKPGQSPKLLIYKVSNRFSGVPDRFSGSGSGTDFTLKISRVEAEDLGVYYCFQTSHVPYTFGGGTKLEIK

SEQ ID NO: 155 - protein, 120 aa, Artificial Sequence
  Synthetic Construct
QVHLKESGPGLVAPSQSLSITCTVSGFSLTGYGVNWIRQPPGKNLEWLGMIWGDGSTDYNSVLKSRLSISKDNAKSQVFLEMNSLQTDDTANYYCARDRGISYYYAMDYWGQGTSVTVSS

SEQ ID NO: 156 - protein, 111 aa, Artificial Sequence
  Synthetic Construct
DIVLTQSPASLAVSLGQRATISCRASKSVSASGYSYLHWYQQKPGQPPKLLIFLAYNLGSGVPARFSGSGAGTDFTLNIHPVEEEDAATYYCQHSREFPLTFGAGTKLELK

SEQ ID NO: 157 - protein, 21 aa, Artificial Sequence
  Synthetic Construct
MALPVTALLLPLALLLHAARP

SEQ ID NO: 158 - protein, 15 aa, Artificial Sequence
  Synthetic Construct
GGGGSGGGGSGGGGS

SEQ ID NO: 159 - protein, 69 aa, Artificial Sequence
  Synthetic Construct
TTTPAPRPPTPAPTIASQPLSLRPEACRPAAGGAVHTRGLDFACDIYIWAPLAGTCGVLLLSLVITLYC

SEQ ID NO: 160 - protein, 42 aa, Artificial Sequence
  Synthetic Construct
KRGRKKLLYIFKQPFMRPVQTTQEEDGCSCRFPEEEEGGCEL

SEQ ID NO: 161 - protein, 41 aa, Artificial Sequence
  Synthetic Construct
RSKRSRLLHSDYMNMTPRRPGPTRKHYQPYAPPRDFAAYRS

SEQ ID NO: 162 - protein, 112 aa, Artificial Sequence
  Synthetic Construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 593 aa, Artificial Sequence
  Synthetic Construct
TAAGGTCTCTAAAATTCCGCCCCCCCCCTAACGTTACTGGCCGAAGCCGCTTGGAATAAGGCCGGTGTGCGTTTGTCTATATGTTATTTTCCACCATATTGCCGTCTTTTGGCAATGTGAGGGCCCGGAAACCTGGCCCTGTCTTCTTGACGAGCATTCCTAGGGGTCTTTCCCCTCTCGCCAAAGGAATGCAAGGTCTGTTGAATGTCGTGAAGGAAGCAGTTCCTCTGGAAGCTTCTTGAAGACAAACAACGTCTGTAGCGACCCTTTGCAGGCAGCGGAACCCCCCACCTGGCGACAGGTGCCTCTGCGGCCAAAAGCCACGTGTATAAGATACACCTGCAAAGGCGGCACAACCCCAGTGCCACGTTGTGAGTTGGATAGTTGTGGAAAGAGTCAAATGGCTCTCCTCAAGCGTATTCAACAAGGGGCTGAAGGATGCCCAGAAGGTACCCCATTGTATGGGATCTGATCTGGGGCCTCGGTACACATGCTTTACATGTGTTTAGTCGAGGTTAAAAAACGTCTAGGCCCCCCGAACCACGGGGACGTGGTTTTCCTTTGAAAAACACGATGATAATATGGCCACAACC

SEQ ID NO: 164 - protein, 237 aa, Artificial Sequence
  Synthetic Construct
MVSKGEELIKENMHMKLYMEGTVNNHHFKCTSEGEGKPYEGTQTMRIKVVEGGPLPFAFDILATSFMYGSRTFIKYPKGIPDFFKQSFPEGFTWERVTTYEDGGVVTVMQDTSLEDGCLVYNVKIRGVNFPSNGPVMQKKTLGWEANTEMLYPADGGLEGRSDMALKLVGGGHLSCSFVTTYRSKKPAKNLKMPGIHAVDHRLERLEESDNEMFVVQREHAVARYCDLPSKLGHKLN

SEQ ID NO: 165 - protein, 983 aa, Homo sapiens
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 166 - protein, 521 aa, Homo sapiens
ELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQ

SEQ ID NO: 167 - protein, 24 aa, Homo sapiens
VVMIAISAAVAIILLTVVIYVLIG

SEQ ID NO: 168 - protein, 418 aa, Homo sapiens
RFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 169 - protein, 179 aa, Homo sapiens
EVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVK

SEQ ID NO: 170 - protein, 111 aa, Homo sapiens
PPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQA

SEQ ID NO: 171 - protein, 96 aa, Homo sapiens
APSPVLTIKKDRTSRNSISLSWQEPEHPNGIILDYEVKYYEKQEQETSYTILRARGTNVTISSLKPDTIYVFQIRARTAAGYGTNSRKFEFETSPD

SEQ ID NO: 172 - protein, 96 aa, Homo sapiens; identical to SEQ ID NO: 171

SEQ ID NO: 173 - protein, 262 aa, Homo sapiens
ISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLI

SEQ ID NO: 174 - protein, 65 aa, Homo sapiens
TTFRTTGDWLNGVWTAHCKEIFTGVEYSSCDTIAKISTDDMKKVGVTVVGPQKKIISSIKALETQ

SEQ ID NO: 175 - protein, 330 aa, Homo sapiens [sequence omitted]

SEQ ID NO: 176 - protein, 98 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 177 - protein, 12 aa, Artificial Sequence
  Synthetic Construct
EPKSCDKTHTCP

SEQ ID NO: 178 - protein, 113 aa, Artificial Sequence
  Synthetic Construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 179 - protein, 107 aa, Artificial Sequence
  Synthetic Construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPEL

SEQ ID NO: 180 - protein, 213 aa, Artificial Sequence
  Synthetic Construct
MRPGTGQGGLEAPGEPGPNLRQRWPLLLLGLAVVTHGLLRPTAASQSRALGPGAPGGSSRSSLRSRWGRFLLQRGSWTGPRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 181 - protein, 9 aa, Cytomegalovirus
VTEHDTLLY

SEQ ID NO: 182 - protein, 11 aa, Cytomegalovirus
YSEHPTFTSQY

SEQ ID NO: 183 - protein, 9 aa, Cytomegalovirus
NLVPMVATV

SEQ ID NO: 184 - protein, 9 aa, Cytomegalovirus
VLEETSVML

SEQ ID NO: 185 - protein, 10 aa, Cytomegalovirus
YILEETSVML

SEQ ID NO: 186 - protein, 10 aa, Cytomegalovirus
AYAQKIFKIL

SEQ ID NO: 187 - protein, 9 aa, Cytomegalovirus
QYDPVAALF

SEQ ID NO: 188 - protein, 10 aa, Cytomegalovirus
TPRVTGGGAM

SEQ ID NO: 189 - protein, 11 aa, Cytomegalovirus
RPHERNGFTVL

SEQ ID NO: 190 - protein, 9 aa, Cytomegalovirus
ELRRKMMYM

SEQ ID NO: 191 - protein, 9 aa, Cytomegalovirus
ELKRKMIYM

SEQ ID NO: 192 - protein, 9 aa, Cytomegalovirus
QIKVRVDMV

SEQ ID NO: 193 - protein, 9 aa, Cytomegalovirus
DELRRKMMY

SEQ ID NO: 194 - protein, 9 aa, Cytomegalovirus
IPSINVHHY

SEQ ID NO: 195 - protein, 12 aa, Cytomegalovirus
CPSQEPMSIYVY

SEQ ID NO: 196 - protein, 9 aa, Cytomegalovirus
CEDVPSGKL

SEQ ID NO: 197 - protein, 9 aa, Cytomegalovirus
HERNGFTVL

SEQ ID NO: 198 - protein, 9 aa, Cytomegalovirus
EEAIVAYTL

SEQ ID NO: 199 - protein, 11 aa, Cytomegalovirus
QEFFWDANDIY

SEQ ID NO: 200 - protein, 9 aa, Cytomegalovirus
TRATKMQVI

SEQ ID NO: 201 - protein, 9 aa, Cytomegalovirus
YAYIYTTYL

SEQ ID NO: 202 - protein, 9 aa, Cytomegalovirus
QAIRETVEL

SEQ ID NO: 203 - protein, 9 aa, Cytomegalovirus
CRVLCCYVL

SEQ ID NO: 204 - protein, 12 aa, Cytomegalovirus
HELLVLVKKAQL

SEQ ID NO: 205 - protein, 11 aa, Cytomegalovirus
AYSNTHSTRYV

SEQ ID NO: 206 - protein, 15 aa, Cytomegalovirus
QEFFWDANDIYRIFA

SEQ ID NO: 207 - protein, 15 aa, Cytomegalovirus
CMLTITTARSKYPYH

SEQ ID NO: 208 - protein, 15 aa, Cytomegalovirus
PLKMLNIPSINVHHY

SEQ ID NO: 209 - protein, 15 aa, Cytomegalovirus
EHPTFTSKYRIQGKL

SEQ ID NO: 210 - protein, 15 aa, Artificial Sequence
  Synthetic Construct
AGILARNLVPMVATV

SEQ ID NO: 211 - protein, 10 aa, Cytomegalovirus
KARAKKDELR

SEQ ID NO: 212 - protein, 107 aa, Artificial Sequence [sequence omitted]

SEQ ID NO: 213 - protein, 547 aa, Artificial Sequence
  Synthetic Construct
MGWSCIILFLVATATGVHSELIPQPSNEVNLLDSKTIQGELGWISYPSHGWEEISGVDEHYTPIRTYQVCNNVMDHFQNNWLRTNWVPRNSAQKIYVELKFTLRDCNSIPLVLGTCKETFNLYYMESDDDHGVKFREHQFFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCACPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQHHHHHH

SEQ ID NO: 214 - DNA, 1666 bp, Artificial Sequence
  Synthetic Construct
gaattcccgccgccaccatgggctggtcctgcatcattctgtttctggtggccacagccaccggcgtgcactctgaactgattccccagcctagcaacgaagtgaacctgctggacagcaagaccatccagggcgaactcggctggatcagctatccttctcacggctgggaagagatcagcggcgtggacgagcactacacccctatcagaacctaccaagtgtgcaacgtgatggaccacagccagaacaactggctgcggaccaattgggtgccccggaatagcgccagaaaatctacgtggaactgaagttcaccctgcgggactgcaacagcatccctctggttctgggcacctgtaaagagacattcaacctgtactacatggaaagcgacgacgaccacggcgtgaagtttagagagcaccagttcaccaagatcgacacaatcgccgccgacgagagcttcacccagatggatctgggcgacacagaatcctgaagctgaacaccgagatcagagaagtgggccccgtgaacaagaagggcttctacctggccttccaggatgtgggagcttgtgtggctctggtgtctgtgcgggtgtacttcaagaaatgccccttcaccgtgaagaacctggccatgtttcccgacaccgtgcctatggatagccagagcctggtggaagtgcggggcagctgtgtgaacaacagcaaagaagaggaccctcctcggatgtactgcagcaccgaaggcgaatggctggtgcctatcggcaagtgctcttgcaatgccggctatgaggaacggggcttcatgtgccaggcctgcagacccggcttttacaaggccctggacggcaacatgaagtgcgccaagtgtcctcctcacagcagcacccaagaggacggctccatgaactgcagatgcgagaacaactacttccgggccgacaaggacccacctagcatggcctgtacacggcctccaagcagccccagaaacgtgatcagcaacatcaacgagacaagcgtgatcctggattggagctggcccctggatacaggcggcagaaaggacgtgaccttcaacatcatctgcaagaagtgcggctggaacatcaagcagtgcgagccttgcagccccaacgtgcggtttctgcctagacagttcggcctgaccaacaccaccgtgaccgtgacagatctgctggcccacaccaactacaccttcgagatcgacgccgtgaacggcgtgtcagagcttagctctccacctaggcagtttgccgccgtgtccatcaccaccaatcaggctgctccatctcctgtgctgaccatcaagaaggaccggaccagccggaacagcatcagcctgtcttggcaagagcctgagcaccccaacggcatcatcctggactatgaagtgaagtactacgagaagcaagagcaagagacaagctacaccatcctgagagccagaggcaccaacgtgacaatcagctccctgaagcctgacaccatctacgtgttccagatccgggccagaacagccgccggatacggcaccaatagccggaagttcgagttcgagactagccccgacagcttcagcatctctggcgagtctagccagcaccaccaccatcaccattgataagctt

SEQ ID NO: 215 - DNA, 20 bp, Artificial Sequence
  Forward Primer
cagcggctacacctttacca

SEQ ID NO: 216 - DNA, 20 bp, Artificial Sequence
  Reverse Primer
ccggagaatctatccggcac